SEQUENCE LISTING (12 sequences)

SEQ ID NO: 1 - DNA, 3358 bp, Homo sapiens
gagctggagcagccgccaccgccgccgccgagggagccccgggacggcagcccctgggcgcagggtgcgctgttctcggagtccgacccagggcgactcacgcccactggtgcgacccggacagcctgggactgacccgccggcccaggcgaggctgcagccagagggctgggaagggatcgcgctcgcggcatccagaggcggccaggcggaggcgagggagcaggttagagggacaaagagctttgcagacgtccccggcgtcctgcgagcgccagcggccgggacgaggcggccgggagcccgggaagagcccgtggatgttctgcgcgcggcctgggagccgccgccgccgccgcctcagcgagaggaggaatgcaccggccgcgccgccgcgggacgcgcccgccgctcctggcgctgctggccgcgctgctgctggccgcacgcggggctgctgcccaagaaacagagctgtcagtcagtgctgaattagtgcctacctcatcatggaacatctcaagtgaactcaacaaagattcttacctgacccttgatgaaccaatgaataacatcaccacgtctctgggccagacagcagaactgcactgcaaagtctctgggaatccacctcccaccatccgctggttcaaaaatgatgctcctgtggtccaggagccccggaggctctcctttcggtccaccatctatggctctcggctgcggattagaaacctcgacaccacagacacaggctacttccagtgcgtggcaacaaacggcaaggaggtggtttcttccactggagtcttgtttgtcaagtttggcccccctcccactgcaagtccaggatactcagatgagtatgaagaagatggattctgtcagccatacagagggattgcatgtgcaagatttattggcaaccgcaccgtctatatggagtctttgcacatgcaaggggaaatagaaaatcagatcacagctgccttcactatgattggcacttccagtcacttatctgataagtgttctcagttcgccattccttccctgtgccactatgccttcccgtactgcgatgaaacttcatccgtcccaaagccccgtgacttgtgtcgcgatgaatgtgaaatcctggagaatgtcctgtgtcaaacagagtacatttttgcaagatcaaatcccatgattctgatgaggctgaaactgccaaactgtgaagatctcccccagccagagagcccagaagctgcgaactgtatccggattggaattcccatggcagatcctataaataaaaatcacaagtgttataacagcacaggtgtggactaccgggggaccgtcagtgtgaccaaatcagggcgccagtgccagccatggaattcccagtatccccacacacacactttcaccgcccttcgtttcccagagctgaatggaggccattcctactgccgcaacccagggaatcaaaaggaagctccctggtgcttcaccttggatgaaaactttaagtctgatctgtgtgacatcccagcttgcgattcaaaggattccaaggagaagaataaaatggaaatcctgtacatactagtgccaagtgtggccattcccctggccattgctttactcttcttcttcatttgcgtctgtcggaataaccagaagtcatcgtcggcaccagtccagaggcaaccaaaacacgtcagaggtcaaaatgtggagatgtcaatgctgaatgcatataaacccaagagcaaggctaaagagctacctctttctgctgtacgctttatggaagaattgggtgagtgtgcctttggaaaaatctataaaggccatctctatctcccaggcatggaccatgctcagctggttgctatcaagaccttgaaagactataacaacccccagcaatggatggaatttcaacaagaagcctccctaatggcagaactgcaccaccccaatattgtctgccttctaggtgccgtcactcaggaacaacctgtgtgcatgctttttgagtatattaatcagggggatctccatgagttcctcatcatgagatccccacactctgatgttggctgcagcagtgatgaagatgggactgtgaaatccagcctggaccacggagattttctgcacattgcaattcagattgcagctggcatggaatacctgtctagtcacttctttgtccacaaggaccttgcagctcgcaatattttaatcggagagcaacttcatgtaaagatttcagacttggggctttccagagaaatttactccgctgattactacagggtccagagtaagtccttgctgcccattcgctggatgccccctgaagccatcatgtatggcaaattctcttctgattcagatatctggtcctttggggttgtcttgtgggagattttcagttttggactccagccatattatggattcagtaaccaggaagtgattgagatggtgagaaaacggcagctcttaccatgctctgaagactgcccacccagaatgtacagcctcatgacagagtgctggaatgagattccttctaggagaccaagatttaaagatattcacgtccggcttcggtcctgggagggactctcaagtcacacaagctctactactccttcagggggaaatgccaccacacagacaacctccctcagtgccagcccagtgagtaatctcagtaaccccagatatcctaattacatgttcccgagccagggtattacaccacagggccagattgctggtttcattggcccgccaatacctcagaaccagcgattcattcccatcaatggatacccaatacctcctggatatgcagcgtttccagctgcccactaccagccaacaggtcctcccagagtgattcagcactgcccacctcccaagagtcggtccccaagcagtgccagtgggtcgactagcactggccatgtgactagcttgccctcatcaggatccaatcaggaagcaaatattcctttactaccacacatgtcaattccaaatcatcctggtggaatgggtatcaccgtttttggcaacaaatctcaaaaaccctacaaaattgactcaaagcaagcatctttactaggagacgccaatattcatggacacaccgaatctatgatttctgcagaactgtaaaatgcacaacttttgtaaatgtggtatacaggacaaactagacggccgtagaaaagatttatattcaaatgtttttattaaagtaaggttctcatttagcagacatcgcaacaagtaccttctgtgaagtttcactgtgtcttaccaagcaggacagacactcggccag

SEQ ID NO: 2 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWMEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 3 - DNA, 3765 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtg

SEQ ID NO: 4 - DNA, 1570 bp, Homo sapiens
ccggcgcagcgcggccgcagcagcctccgccccccgcacggtgtgagcgcccgccgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaatgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggtttgagctgaattatcacatgaatataaatgggaaatcagtgttttagagagagaacttttcgacatatttcctgttcccttggaataaaaacatttcttctgaaattttaccgttaa

SEQ ID NO: 5 - DNA, 3155 bp, Homo sapiens
aagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagttaccacctcctccccggccggcggcggacagtggacgcggcggcgagccgcgggcaggggccggagcccgcccccggaggcggggtggagggggtcggagctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagcgcgcgcgctccccagccctggcccggcctcgggccgggaggaagagtagctcgccgaggcgccgaggagagcgggccgccccacagcccgagccggagagggacgcgagccgcgcgccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctccaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacacactcgcgttgcaaggcgaggcagcttgagttaaacgaacgtacttgcagatgtgacaagccgaggcggtgagccgggcaggaggaaggagcctccctcagggtttcgggaaccagatctctctccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaagaatgtgcaaggccagggcatgggggcaaatatgacccagttttgggaacaccgacaaacccagccctggcgctgagcctctctaccccaggtcagacggacagaaagacaaatcacaggttccgggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggaggccactggcagatgtcccggcgaagagaagagacacattgttggaagaagcagcccatgacagcgccccttcctgggactcgccctcatcctcttcctgctccccttcctggggtgcagcctaaaaggacctatgtcctcacaccattgaaaccactagttctgtccccccaggaaacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattagaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaagagggtttcaatatacatctacatactatatatatattgggcaacttgtatttgtgtgtatatatatatatatatgtttatgtatatatgtgatcctgaaaaaataaacatcgctattctgttttttatatgttcaaaccaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatctta

SEQ ID NO: 6 - DNA, 3475 bp, Homo sapiens
cgaggcggcatccgagggctgggccggcgccctgggggaccccgggctccggaggccatgccggcgttggcgcgcgacgcgggcaccgtgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgatgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtaggaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtagaggaacaatttagttttaaggacttcatccctccacctatccctaacaggctgtagattaccaaaacaagattaatttcatcactaaaagaaaatctattatcaactgctgcttcaccagacttttctctagaagccgtctgcgtttactcttgttttcaaagggacttttgtaaaatcaaatcatcctgtcacaaggcaggaggagctgataatgaactttattggagcattgatctgcatccaaggccttctcaggccggcttgagtgaattgtgtacctgaagtacagtatattcttgtaaatacataaaacaaaagcattttgctaaggagaagctaatatgattttttaagtctatgttttaaaataatatgtaaatttttcagctatttagtgatatattttatgggtgggaataaaatttctactacag

SEQ ID NO: 7 - DNA, 1601 bp, Homo sapiens
aagtgctgggattacaggtgtgagccagggcaccaggcttagatgtggctctttggggagataattttgtccagagacctttctaacgtattcatgccttgtatttgtacagcattaatctggtaattgattattttaatgtaaccttgctaaaggagtgatttctatttcctttcttaaagaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccgaagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaa

SEQ ID NO: 8 - DNA, 2320 bp, Homo sapiens
cccgggccagggtccacctgtccccgcagcgccggctcgcgccctcctgccgcagccaccgagccgccgtctagcgccccgacctcgccaccatgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggtgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtattattctgcagcatgacctgtgaccagcactgtctcagtttcactttcacatagatgtccctttcttggccagttatcccttccttttagcctagttcatccaatcctcactgggtggggtgaggaccactccttacactgaatatttatatttcactatttttatttatatttttgtaattttaaataaaagtgatcaataaaatgtgatttttctgatgaa

SEQ ID NO: 9 - DNA, 2876 bp, Homo sapiens
gaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagc

SEQ ID NO: 10 - DNA, 7108 bp, Homo sapiens
aaaactgcgactgcgcggcgtgagctcgctgagacttcctggaccccgcaccaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagcccaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaggaaaatgaaactaggaaaggtttcttaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaaca

SEQ ID NO: 11 - DNA, 10987 bp, Homo sapiens
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagtattattttacaatcaacaaaatggtcatccaaactcaaacttgagaaaatatcttgctttcaaattgacacta

SEQ ID NO: 12 - DNA, 3670 bp, Homo sapiens
ggctagcgcgggaggtggagaaagaggcttgggcggccccgctgtagccgcgtgtgggaggacgcacgggcctgcttcaaagctttgggataacagcgcctccgggggataatgaatgcggagcctccgttttcagtcgacttcagatgtgtctccacttttttccgctgtagccgcaaggcaaggaaacatttctcttcccgtactgaggaggctgaggagtgcactgggtgttcttttctcctctaacccagaactgcgagacagaggctgagtccctgtaaagaacagctccagaaaagccaggagagcgcaggagggcatccgggaggccaggaggggttcgctggggcctcaaccgcacccacatcggtcccacctgcgagggggcgggacctcgtggcgctggaccaatcagcacccacctgcgctcacctggcctcctcccgctggctcccgggggctgcggtgctcaaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatagtgaccgaccagaatgaccacaagcccaagtttacccaggacaccttccgagggagtgtcttagagggagtcctaccaggtacttctgtgatgcagatgacagccacagatgaggatgatgccatctacacctacaatggggtggttgcttactccatccatagccaagaaccaaaggacccacacgacctcatgttcacaattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaacgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaaaggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctaggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttccc

(19) United States
(12) Patent Application Publication
(10) Pub. No.: US 2008/0318212 A1
(43) Pub. Date: Dec. 25, 2008

(54) Orphan Receptor Tyrosine Kinase as a Target in Breast Cancer

(76) Inventors: Cindy A. Wilson, Santa Monica, CA (US); Judy Dering, Thousand Oaks, CA (US); Dennis J. Slamon, Woodland Hills, CA (US)

Correspondence Address: GATES & COOPER LLP;HOWARD HUGHES CENTER, 6701 CENTER DRIVE WEST, SUITE 1050, LOS ANGELES, CA, 90045, US

(21) Appl. No.: 11/547,934

(22) PCT Filed: Apr. 6, 2005
(86) PCT No.: PCT/US05/11425

§ 371 Date: Oct. 6, 2006

Related U.S. Application Data
(60) Provisional application No. 60/559,762, filed on Apr. 6, 2004.

(51) Int. Cl. C12Q 1/68; G01N 33/574

(52) U.S. Cl. 435/6; 435/7.23

(57) ABSTRACT

Methods and materials relating to the orphan receptor tyrosine kinase (ROR1) are described. ROR1 exhibits restricted tissue expression in normal adult tissue and is overexpressed in certain breast cancer subtypes. ROR1 provides a diagnostic and/or therapeutic target for breast cancers.

### CROSS REFERENCE TO RELATED APPLICATIONS

This application claims priority under Section 119(e) from U.S. Provisional Application Ser. No. 60/559,762 filed Apr. 6, 2004, the contents of which are incorporated herein by reference.

### FIELD OF THE INVENTION

The invention described herein relates to methods and compositions useful in the diagnosis, treatment and management of cancers that express orphan receptor tyrosine kinase (ROR1), particularly breast cancers.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise.

Worldwide, several cancers stand out as the leading killers. In particular, carcinomas of the breast, lung, prostate, colon, pancreas, and ovary represent the primary causes of cancer death. These and virtually all other carcinomas share a common lethal feature. With very few exceptions, metastatic disease from a carcinoma is fatal. Moreover, even for those cancer patients who initially survive their primary cancers, common experience has shown that their lives are dramatically altered and many cancer patients experience a recurrence.

Cancers of the breast are one of the leading causes of death among women, with the cumulative lifetime risk of a woman developing breast cancer estimated to be 1 in 9. Consequently, understanding the origins and subtypes of these malignancies as well as models for the identification of new diagnostic and therapeutic modalities is of significant interest to health care professionals. Most women that die from breast cancer succumb not to the original primary disease, which is usually amenable to various therapies, but rather from metastatic spread of the breast cancer to distant sites. This fact underscores the need to develop both additional diagnostic methods as well as novel anticancer agents or more aggressive forms of therapy directed specifically against breast tumor subtypes.

### SUMMARY OF THE INVENTION

The present invention relates to the gene designated orphan receptor tyrosine kinase ROR1, which is aberrantly-expressed in cancers including cancers of the breast. Breast cancer tumors that overexpress ROR1 are associated with a poor prognosis anal the percentage of poor prognosis tumors in the ROR1 group (70% of sporadic) is higher than for any other single prognostic gene analyzed including Her-2, epidermal growth factor receptor (EGFR), vascular endothelial cell growth factor (VEGF), Fms-like tyrosine kinase-3 (Flt3), C-MYC, urokinase plasminogen activator (uPA) and plasminogen activator inhibitor 1 (PAI-1). Moreover, cancers of the breast can be grouped into a number of distinct subtypes and ROR1 is specifically upregulated in the basal and BRCA 1 subtypes. The expression profile of ROR1 in normal adult tissues, combined with the aberrant-expression observed in various breast cancer subtypes, demonstrate that ROR1 can serve as a useful diagnostic target for such cancers.

The invention provides polynucleotides corresponding or complementary to all or part of ROR1 genes, mRNAs, and/or coding sequences, preferably in isolated forms including polynucleotides encoding ROR1 proteins and fragments thereof, DNA, RNA, DNA/RNA hybrid, and related molecules, polynucleotides or oligonucleotides complementary to the ROR1 genes or mRNA sequences or parts thereof, and polynucleotides or oligonucleotides that hybridize to the ROR1 genes, mRNAs, or to ROR1-encoding polynucleotides. Also provided are means for isolating cDNAs and the genes encoding ROR1. Recombinant DNA molecules containing ROR1 polynucleotides, cells transformed or transduced with such molecules, and host-vector systems for the expression of ROR1 gene products are also provided. The invention further provides ROR1 proteins and polypeptide fragments thereof. The invention further provides antibodies that bind to ROR1 proteins and polypeptide fragments thereof, including polyclonal and monoclonal antibodies, murine and other mammalian antibodies, chimeric antibodies, humanized and fully human antibodies, and antibodies labeled with a detectable marker.

The invention further provides methods for detecting the presence and status of ROR1 polynucleotides and proteins in various biological samples (e.g. breast cancer biopsies), as well as methods for identifying cells that express ROR1. A typical embodiment of this invention provides methods for monitoring ROR1 gene products in a tissue sample having or suspected of having some form of growth disregulation such as that found in various breast cancers, for example the basal and BRCA 1 subtypes as described in Sorlie et al., PNAS (2001), 98(19): 10869-10874, which is incorporated herein by reference.

An illustrative embodiment of the invention is a method of examining a test biological sample comprising a human breast cell for evidence of altered cell growth that is indicative of a breast cancer by evaluating the levels of orphan receptor tyrosine kinase (ROR1) polynucleotides that encode the ROR1 polypeptide shown in SEQ ID NO: 2 in the biological sample, wherein an increase in the levels of the ROR1 polynucleotides in the test sample relative to a normal breast tissue sample provide evidence of altered cell growth that is indicative of a breast cancer; and wherein the levels of the ROR1 polynucleotides in the cell are evaluated by contacting the sample with a ROR1 complementary polynucleotide that hybridizes to a ROR1 nucleotide sequence shown in SEQ ID NO: 1, or a complement thereof, and evaluating the presence of a hybridization complex formed by the hybridization of the ROR1 complementary polynucleotide with the ROR1 polynucleotides in the test biological sample. In certain embodiments of the invention, the breast cancer is of the basal subtype. In other embodiments of the invention, the breast cancer is of the BRCA1 subtype.

A related embodiment is a method of examining a human breast cell for evidence of altered cell growth that is associated with or provides evidence of a breast cancer by evaluating the levels of orphan receptor tyrosine kinase (ROR1) polynucleotides that encode the ROR1 polypeptide shown in SEQ ID NO: 2 in the human breast cell, wherein an increase in the levels of the ROR1 polynucleotides (e.g. mRNAs and genomic sequences) in the human breast cell relative to a normal human breast cell provides evidence of altered cell growth that is associated with or provides evidence of a breast cancer; and wherein the levels of the ROR1 polynucleotides in the human breast cell are evaluated by contacting the endogenous ROR1 polynucleotide sequences in the human breast cell with a ROR1 complementary polynucleotide the ROR1 complementary polynucleotide (e.g. a probe labelled with a detectable marker or a PCR primer) and which specifically hybridizes to a ROR1 nucleotide sequence shown in SEQ ID NO: 1 and evaluating the presence of a hybridization complex formed by the hybridization of the ROR1 complementary polynucleotide with the ROR1 polynucleotides in the sample (e.g. via Northern analysis or PCR) so that evidence of altered cell growth that is associated with or provides evidence of a breast cancer is examined. Certain embodiments of the invention further include the step of examining the expression and/or sequences of Her-2 (SEQ ID NO: 3), EGFR (SEQ ID NO: 4), VEGF (SEQ ID NO: 5),

FMS-like tyrosine kinase (SEQ ID NO: 6), MYC (SEQ ID NO: 7), urokinase plasminogen activator (SEQ ID NO: 8), plasminogen activator inhibitor (SEQ ID NO: 9), BRCA1 (SEQ ID NO: 10) or BRCA2 (SEQ ID NO: 11) polynucleotides or polypeptides in the test biological sample.

Another embodiment of the invention is a method of examining a test biological sample comprising a human breast cell for evidence of altered cell growth that is indicative of a breast cancer, the method comprising evaluating the levels of orphan receptor tyrosine kinase (ROR1) polypeptides having the sequence shown in SEQ ID NO: 2 in the biological sample, wherein an increase in the levels of the ROR1 polypeptides in the test sample relative to a normal breast tissue sample provide evidence of altered cell growth that is indicative of a breast cancer; and wherein the levels of the ROR1 polypeptides in the cell are evaluated by contacting the sample with an antibody that immunospecifically binds to a ROR1 polypeptide sequence shown in SEQ ID NO: 2 and evaluating the presence of a complex formed by the binding of the antibody with the ROR1 polypeptides in the sample.

A related embodiment of the invention is a method of examining a human breast cell (e.g. from a biopsy) that is suspected of being cancerous for evidence of altered cell growth that is indicative of a breast cancer, the method comprising evaluating the levels of orphan receptor tyrosine kinase (ROR1) polypeptides having the sequence shown in SEQ ID NO: 2 in the breast cell, wherein an increase in the levels of the ROR1 polypeptides in the human breast cell relative to a normal breast cell (e.g. a normal cell from the individual providing the human breast cell) provide evidence of altered cell growth that is indicative of a breast cancer; and wherein the levels of the ROR1 polypeptides in the cell are evaluated by contacting the sample with an antibody (e.g. one labelled with a detectable market) that immunospecifically binds to a ROR1 polypeptide sequence shown in SEQ ID NO: 2 and evaluating the presence of a complex formed by the binding of the antibody with the ROR1 polypeptides in the sample. Typically the presence of a complex is evaluated by a method selected from the group consisting of ELISA analysis, Western analysis and immunohistochemistry. Optionally, the breast cancer is of the basal or the BRCA 1 subtype.

Yet another embodiment of the invention is a method of examining a test human cell for evidence of a chromosomal abnormality that is indicative of a human cancer by comparing orphan receptor tyrosine kinase (ROR1) polynucleotide sequences from band p31 of chromosome 1 in a normal cell to ROR1 polynucleotide sequences from band p31 of chromosome 1, band p31 on chromosome 1 in the test human cell to identify an amplification or an alteration of the ROR1 polynucleotide sequences in the test human cell, wherein an amplification or an alteration of the ROR1 polynucleotide sequences in the test human cell provides evidence of a chromosomal abnormality that is indicative of a human cancer. In such methods chromosome 1, band p31 in the test human cell is typically evaluated by contacting the ROR1 polynucleotide sequences in the test human cell sample with a ROR1 complementary polynucleotide that specifically hybridizes to a ROR1 nucleotide sequence shown in SEQ ID NO: 1, or a complement thereof, and evaluating the presence of a hybridization complex formed by the hybridization of the ROR1 complementary polynucleotide with the ROR1 polynucleotide sequences in the test human cell (e.g. by Northern analysis, Southern analysis or polymerase chain reaction analysis).

Another embodiment of the invention is a kit comprising a container, a label on said container, and a composition contained within said container; wherein the composition includes a ROR1 specific antibody and/or a polynucleotide that hybridizes to a complement of the ROR1 polynucleotide shown in SEQ ID NO: 1 under stringent conditions (or binds to a ROR1 polypeptide encoded by the polynucleotide shown in SEQ ID NO: 1), the label on said container indicates that the composition can be used to evaluate the presence of ROR1 protein, RNA or DNA in at least one type of mammalian cell, and instructions for using the ROR1 antibody and/or polynucleotide for evaluating the presence of ROR1 protein, RNA or DNA in at least one type of mammalian cell.

The invention further provides various therapeutic compositions and strategies for treating cancers that express ROR1 such as breast cancers, including antibody based therapies aimed at inhibiting the function of ROR1.

### BRIEF DESCRIPTION OF THE FIGURES

FIG. 1A shows the complete nucleotide (SEQ ID NO: 1) and FIG. 1B shows the complete amino acid (SEQ ID NO: 2) sequences of ROR1. See e.g. Masiakowski et al., J. Biol. Chem. 267 (36), 26181-26190 (1992); NP_{—}005003 (gi:4826868); and M97675 (gi:337464).

FIG. 2A shows how similar breast cancer subtypes (e.g. having a constellation of shared characteristics) are identified in both the Rosetta/Netherlands (Van't Veet, L. J., et al. (2002) Nature 415, 530-536) and Stanford/Norway (Sotlie et al., Proc Natl Acad Sci USA. 2001 Sep. 11; 98(19):10869-74) data sets. The Rosetta/Netherlands data set is a constrained definition of classes based on expression level of ESR1 and ERBB2, as well as the identification of a BRCA mutation. The Stanford/Norway data set is a cluster-based definition of classes. Markets are a subset of those selected by authors as exemplars for clusters. The expression levels in these data sets are measured a log10 intensity ratio of sample to a reference. FIG. 2B uses different reference RNAs (from those in FIG. 2A) to show a comparison of the profiles (e.g. gene expression patterns, cytological characteristics etc.) of a variety of cell lines selected to represent the wide spectrum of properties found in primary breast cancers.

FIG. 3A shows that ROR1 mRNA expression is specifically upregulated in breast cancer tumors of the basal and BRCA1 subtypes identified in Van't Veer, L. J., et al. (2002) Nature 415, 530-536 (groups 4 and 6). FIG. 3B shows ESR1, HER2 and BRCA1/2 mRNA expression in breast cancer subtypes identified in Van't Veer et al., supra. FIG. 3C provides a schematic showing the expression of ROR1 mRNA as well as a variety of other markers in various cell lines.

FIG. 4A is a scatter plot of ESR1 and ROR1 by prognosis showing that ROR1 expressing tumors are associated with a poor prognosis (metastasis in less than 5 years) in breast cancer subtypes identified in Van't Veer, L. J., et al. (2002) Nature 415, 530-536. Out of 17 overexpressing ROR1 samples, only 3 have a good prognosis. 7 of the samples have BRCA1 mutation but no prognosis data, however BRCA1 mutations is typically associated with a poor outcome. Of the remaining 10 samples, 7 have a poor prognosis. This percentage of poor prognosis for a single gene is the worse of 13 genes studied so far. The percentage (70% of sporadic) of poor prognosis tumors in the ROR1 group is higher than that for any other single prognostic gene analyzed including HER-2, EGFR, VEGF, FLT3, MYC, UPA and PAI. FIG. 4B, is a Scatterplot of HER2 by prognosis showing that fifty-four percent of HER-2 overexpressing tumors are poor prognosis samples. Out of 13 HER overexpressing tumors, 6 are associated with a good prognosis. No BRCA1 samples overexpress HER. Even though all samples are associated with node-negative, early-stage disease, more than 50% of the HER2 samples have poor prognosis.

FIG. 5A shows a Northern blot analysis of ROR1 mRNA expression in a variety of breast cancer cell lines. 5 breast cancer cell lines overexpress ROR1 significantly as compared to normal human mammary epithelial cells (HMECs). ROR1 is also detectable in immortalized HMECs and BT20s. This expression pattern is particularly interesting in that none of the luminal cell lines express detectable ROR1. The overexpressing cell lines have been characterized as either basal or mesenchymal/stromal analogous to the basal tumor group that shows high ROR1 expression. This data confirms the expression of ROR1 in tumor cells. FIG. 5B shows a bar graph of ROR1 mRNA expression by Northern (Phosphoimager units) in a variety of cancer cells. FIG. 5C shows a bar graph of ROR1 mRNA expression by Northern expressed as log ratio (ROR1/mixed reference) in a variety of cancer cells. FIG. 5D shows a bar graph of ROR1 mRNA expression by microarray expressed as log ratio (ROR1/mixed reference) in a variety of cancer cells. FIG. 5E shows comparative graph of ROR1 in RNA expression by Northern versus ROR1 mRNA expression by microarray. FIG. 5F and FIG. 5G show the detection of endogenous ROR1 protein in CAL51 cells using rabbit polyclonal sera (left panels show cells exposed to this anti-ROR1 antibody) with SKBR cells serving as a comparative cell line.

FIG. 6A provides a schematic of ROR1 and related gene expression data in primary tumors generated at UCLA. Briefly, core biopsies from 42 primary breast cancers were snap frozen and assayed. The selection criteria for these biopsies was a tumor>2 cm. The expression profiles utilized 60-mer Agilent oligonucleotide arrays with tumor cRNA labelled with Cy5 Cy3 reference cRNA. FIG. 6B provides a chart of ROR1 expression data in basal, HER-2 overexpressing and luminal cancer subtypes which shows that ROR1 is the best marker of the basal subtype. FIG. 6C provides a graph of ROR1 expression in various cells which shows that ROR1 is exclusively expressed in estrogen receptor (ER) negative breast cancers. FIG. 6D provides a graph of ROR1 expression in various cells which shows that ROR1 is exclusively expressed in basal (androgen receptor negative) breast cancers.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Ausubel et al., eds., 1995, Current Protocols in Molecular Biology, Wiley and Sons). As appropriate, procedures involving the use of commercially available kits and reagents are generally carried out in accordance with manufacturer defined protocols and/or parameters unless otherwise noted.

As used herein, the term “polynucleotide” means a polymeric form of nucleotides of at least about 10 bases or base pairs in length, either ribonucleotides or deoxynucleotides or a modified form of either type of nucleotide, and is meant to include single and double stranded forms of DNA.

As used herein, the term “polypeptide” means a polymer of at least about 6 amino acids. Throughout the specification, standard three letter or single letter designations for amino acids are used.

As used herein, the terms “hybridize”, “hybridizing”, “hybridizes” and the like, used in the context of polynucleotides, ate meant to refer to conventional hybridization conditions, preferably such as hybridization in 50% formamide/6×SSC/0.1% SDS/100 μg/ml ssDNA, in which temperatures for hybridization are above 37 degrees C. and temperatures for washing in 0.1×SSC/0.1% SDS are above 55 degrees C., and most preferably to stringent hybridization conditions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

“Moderately stringent conditions” may be identified as described by Sambrook et al., 1989, Molecular Cloning: A Laboratory Manual, New York: Cold Spring Harbor Press, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of amino acid sequence comparisons, the term “identity” is used to express the percentage of amino acid residues at the same relative positions that are the same. Also in this context, the term “homology” is used to express the percentage of amino acid residues at the same relative positions that are either identical or are similar, using the conserved amino acid criteria of BLAST analysis, as is generally understood in the art. For example, % identity values may be generated by WU-BLAST-2 (Altschul et al., 1996, Methods in Enzymology 266:460-480; blast.wustl/edu/blast/README.html). Further details regarding amino acid substitutions, which are considered conservative under such criteria, are provided below. Additional definitions are provided throughout the subsections that follow.

The following sections describe methods and materials useful in the practice of various embodiments of the invention disclosed herein. The Examples provided below include disclosure that allows the further characterization of the significance of ROR1 in breast cancer subtypes.

### ROR1 Polynucleotides

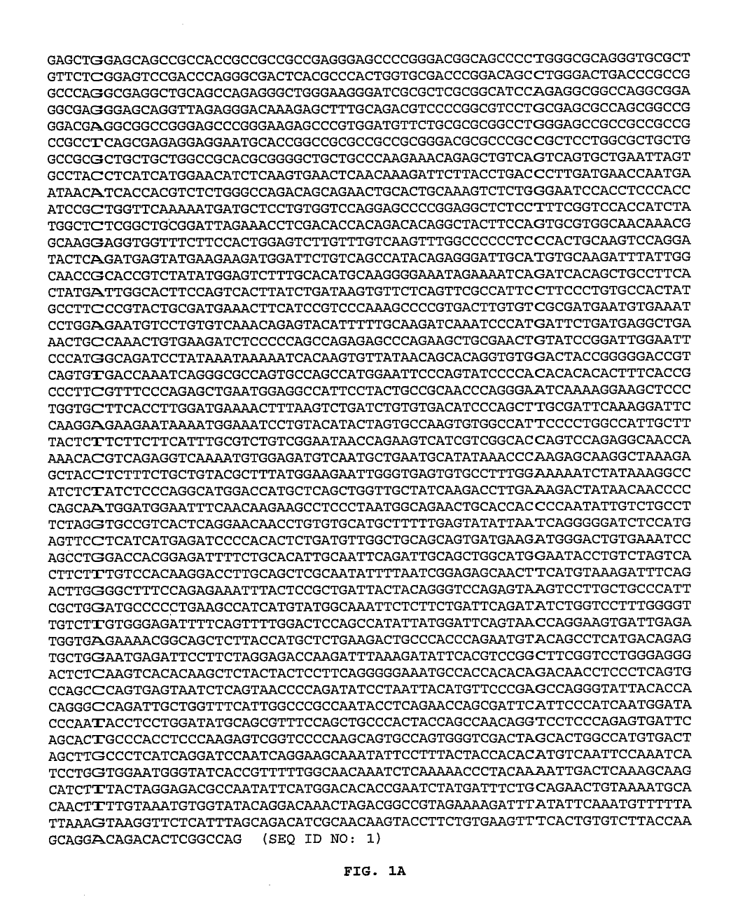

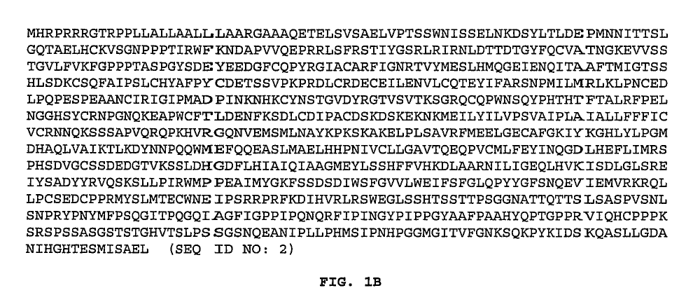

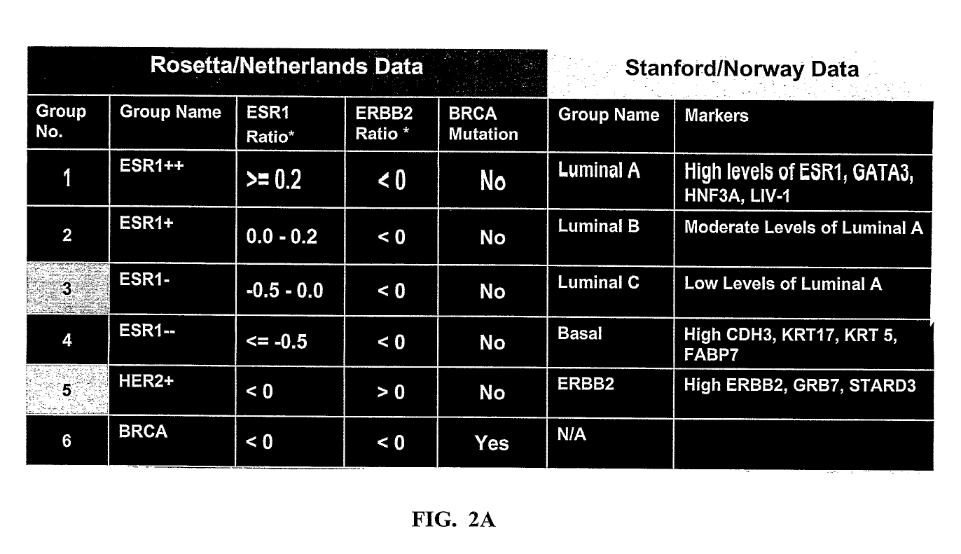

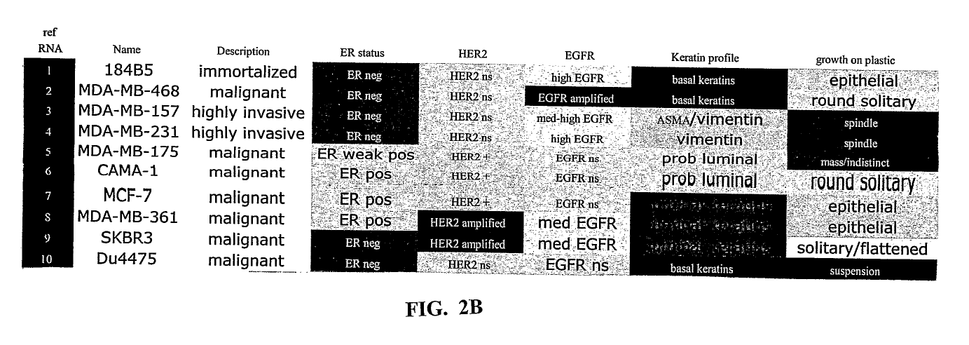

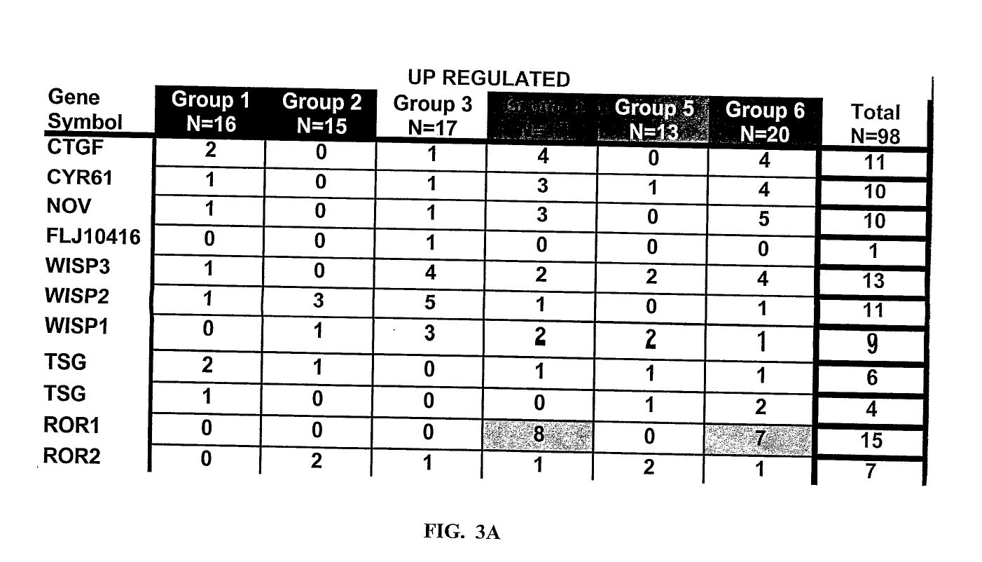

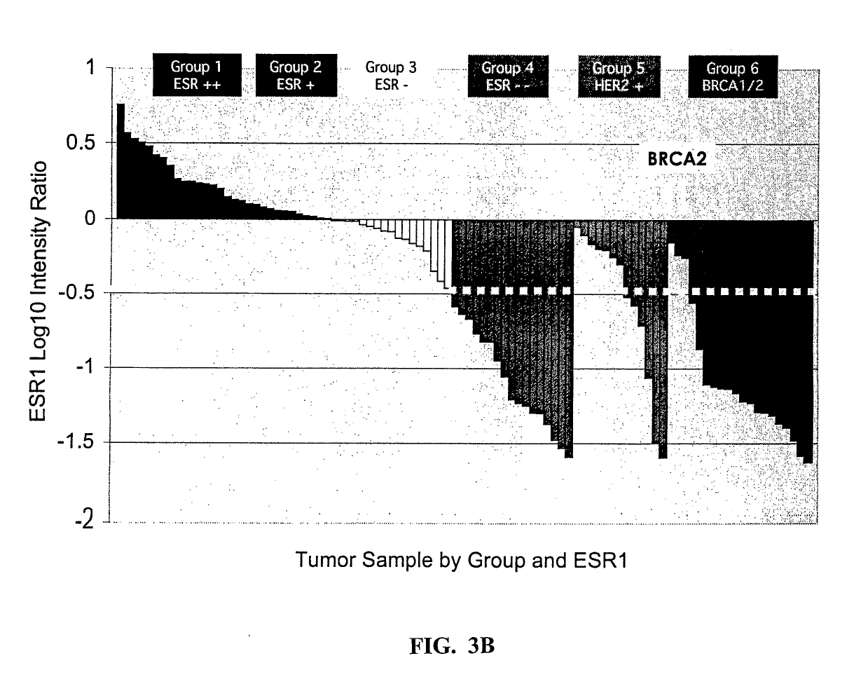

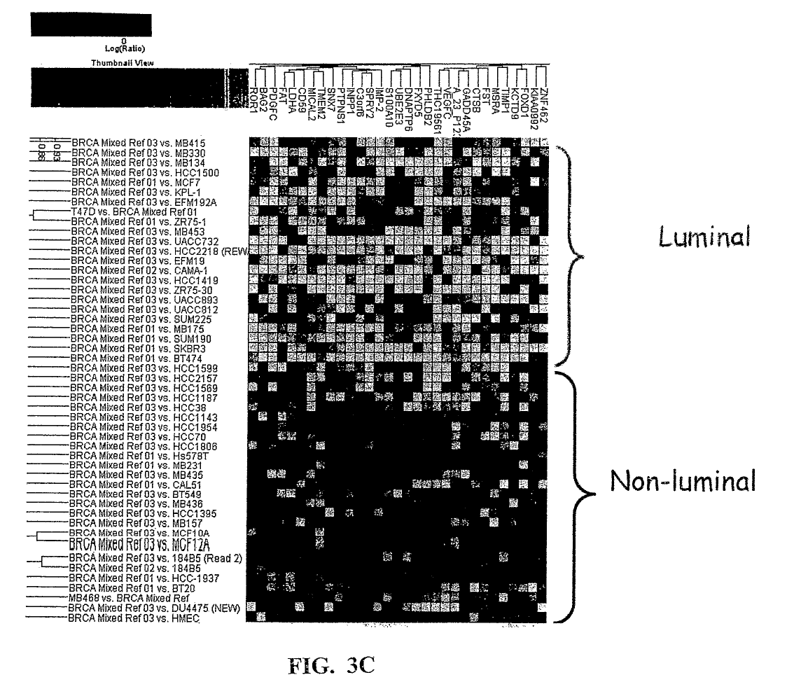

One aspect of the invention provides polynucleotides corresponding or complementary to all or part of a ROR1 gene, mRNA, and/or coding sequence, preferably in isolated form, including polynucleotides encoding a ROR1 protein and fragments thereof, DNA, RNA, DNA/RNA hybrid, and related molecules, polynucleotides or oligonucleotides complementary to a ROR1 gene or mRNA sequence or a part thereof, and polynucleotides or oligonucleotides that hybridize to a ROR1 gene, mRNA, or to a ROR1 encoding polynucleotide (collectively, “ROR1 polynucleotides”). As used herein, the ROR1 gene and protein is meant to include the ROR1 genes and proteins specifically described herein (see, e.g. FIG. 1) and the genes and proteins corresponding to other ROR1 proteins and structurally similar variants of the foregoing. Such other ROR1 proteins and variants will generally have coding sequences that are highly homologous to the ROR1 coding sequence, and preferably will share at least about 80% amino acid identity and at least about 90% amino acid homology (using BLAST criteria), more preferably sharing 95% or greater homology (using BLAST criteria).

One embodiment of a ROR1 polynucleotide is a ROR1 polynucleotide having the sequence shown in FIG. 1. A ROR1 polynucleotide may comprise a polynucleotide having the nucleotide sequence of human ROR1 as shown in FIG. 1, wherein T can also be U; a polynucleotide that encodes all or part of the ROR1 protein; a sequence complementary to the foregoing; or a polynucleotide fragment of any of the foregoing. Another embodiment comprises a polynucleotide having the sequence as shown in FIG. 1, from nucleotide residue number 376 through nucleotide residue number 3189, wherein T can also be U. Another embodiment comprises a polynucleotide that is capable of hybridizing under stringent hybridization conditions to the human ROR1 cDNA shown in FIG. 1 or to a polynucleotide fragment thereof.

Typical embodiments of the invention disclosed herein include ROR1 polynucleotides containing specific portions of the ROR1 mRNA sequence (and those which are complementary to such sequences) such as those that encode the protein and fragments thereof. For example, representative embodiments of the invention disclosed herein include: polynucleotides encoding about amino acid 1 to about amino acid 10 of the ROR1 protein shown in FIG. 1, polynucleotides encoding about amino acid 20 to about amino acid 30 of the ROR1 protein shown in FIG. 1, polynucleotides encoding about amino acid 30 to about amino acid 40 of the ROR1 protein shown in FIG. 1, polynucleotides encoding about amino acid 40 to about amino acid 50 of the ROR1 protein shown in FIG. 1, polynucleotides encoding about amino acid 50 to about amino acid 60 of the ROR1 protein shown in FIG. 1, polynucleotides encoding about amino acid 60 to about amino acid 70 of the ROR1 protein shown in FIG. 1, polynucleotides encoding about amino acid 70 to about amino acid 80 of the ROR1 protein shown in FIG. 1, polynucleotides encoding about amino acid 80 to about amino acid 90 of the ROR1 protein shown in FIG. 1 and polynucleotides encoding about amino acid 90 to about amino acid 100 of the ROR1 protein shown in FIG. 1, etc. Following this scheme, polynucleotides encoding portions of the amino acid sequence of amino acids 100-937 of the ROR1 protein are typical embodiments of the invention. Polynucleotides encoding larger portions of the ROR1 protein are also contemplated. For example polynucleotides encoding from about amino acid 1 (or 20 or 30 or 40 etc.) to about amino acid 20, (or 30, or 40 or 50 etc.) of the ROR1 protein shown in FIG. 1 may be generated by a variety of techniques well known in the art.

Additional illustrative embodiments of ROR1 polynucleotides include embodiments consisting of a polynucleotide having the sequence as shown in FIG. 1 from about nucleotide residue number 1 through about nucleotide residue number 500, from about nucleotide residue number 500 through about nucleotide residue number 1000, from about nucleotide residue number 1000 through about nucleotide residue number 1500, from about nucleotide residue number 1500 through about nucleotide residue number 2000, from about nucleotide residue number 2000 through about nucleotide residue number 2500 and from about nucleotide residue number 2500 through about nucleotide residue number 3358. These polynucleotide fragments can include any portion of the ROR1 sequence as shown in FIG. 1, for example a polynucleotide having the sequence as shown in FIG. 1 from about nucleotide residue number 376 through nucleotide residue number 3189.

The polynucleotides of the preceding paragraphs have a number of different specific uses. For example, because the human ROR1 gene maps to chromosome 1p31.3, polynucleotides encoding different regions of the ROR1 protein can be used to characterize cytogenetic abnormalities on chromosome 1, band p31 that have been identified as being associated with various cancers. In particular, a variety of chromosomal abnormalities in 1p31.3 including loss of heterozygosity have been identified as frequent cytogenetic abnormalities in a number of different cancers (see, e.g., Matthew et al., 1989, Cancer Res. 1994 Dec. 1; 54(23):6265-9; Chunder et al., Pathol Res Pract. 2003; 199(5):313-21. Consequently, polynucleotides encoding specific regions of the ROR1 protein provide new tools that can be used to delineate with a greater precision than previously possible, the specific nature of the cytogenetic abnormalities in this region of chromosome 1 that may contribute to the malignant phenotype. In this context, these polynucleotides satisfy a need in the art for expanding the sensitivity of chromosomal screening in order to identify more subtle and less common chromosomal abnormalities (see, e.g., Evans et al., 1994, Am. J. Obstet. Gynecol. 171(4):1055-1057).

Alternatively, as ROR1 is shown to be aberrantly expressed in breast cancers, in particular the BRCA 1 and basal subtypes, the polynucleotides disclosed herein may be used in methods assessing the status of ROR1 gene products in normal versus cancerous tissues and/or to characterize breast cancer subtypes. Typically, polynucleotides encoding specific regions of the ROR1 protein may be used to assess the levels of ROR1 mRNA in a cell as well as the presence of perturbations (such as deletions, insertions, point mutations etc.) in specific regions of the ROR1 gene products. Exemplary assays include both RT-PCR assays as well as single-strand conformation polymorphism (SSCP) analysis (see, e.g., Marrogi et al., 1999, J. Cutan. Pathol. 26(8): 369-378), both of which utilize polynucleotides encoding specific regions of a protein to examine these regions within the protein.

Other specifically contemplated embodiments of the invention disclosed herein are genomic DNA, cDNAs, ribozymes, and antisense molecules, as well as nucleic acid molecules based on an alternative backbone or including alternative bases, whether derived from natural sources or synthesized. For example, antisense molecules can be RNAs or other molecules, including peptide nucleic acids (PNAs) or non-nucleic acid molecules such as phosphorothioate derivatives, that specifically bind DNA or RNA in a base pair-dependent manner. A skilled artisan can readily obtain these classes of nucleic acid molecules using the ROR1 polynucleotides and polynucleotide sequences disclosed herein.

Antisense technology entails the administration of exogenous oligonucleotides that bind to a target polynucleotide located within the cells. The term “antisense” refers to the fact that such oligonucleotides are complementary to their intracellular targets, e.g., ROR1. See for example, Jack Cohen, 1988, OLIGODEOXYNUCLEOTIDES, Antisense Inhibitors of Gene Expression, CRC Press; and Synthesis 1:1-5 (1988). The ROR1 antisense oligonucleotides of the present invention include derivatives such as S-oligonucleotides (phosphorothioate derivatives or S-oligos, see, Jack Cohen, supra), which exhibit enhanced cancer cell growth inhibitory action. S-oligos (nucleoside phosphorothioates) are isoelectronic analogs of an oligonucleotide (O-oligo) in which a nonbridging oxygen atom of the phosphate group is replaced by a sulfur atom. The S-oligos of the present invention may be prepared by treatment of the corresponding O-oligos with 3H-1,2-benzodithiol-3-one-1,1-dioxide, which is a sulfur transfer reagent. See Iyer, R. P. et al, 1990, J. Org. Chem. 55:4693-4698; and Iyer, R. P. et al., 1990, J. Am. Chem. Soc. 112:1253-1254, the disclosures of which are fully incorporated by reference herein. Additional ROR1 antisense oligonucleotides of the present invention include morpholino antisense oligonucleotides known in the art (see e.g. Partridge et al., 1996, Antisense & Nucleic Acid Drug Development 6: 169-175).

The ROR1 antisense oligonucleotides of the present invention typically may be RNA or DNA that is complementary to and stably hybridizes with the first 100 N-terminal codons or last 100 C-terminal codons of the ROR1 genomic sequence or the corresponding mRNA. While absolute complementarity is not required, high degrees of complementarity are desirable. Use of an oligonucleotide complementary to this region allows for the selective hybridization to ROR1 mRNA and not to mRNA specifying other regulatory subunits of protein kinase. Preferably, the ROR1 antisense oligonucleotides of the present invention are a 15 to 30-mer fragment of the antisense DNA molecule having a sequence that hybridizes to ROR1 mRNA. Optionally, ROR1 antisense oligonucleotide is a 30-mer oligonucleotide that is complementary to a region in the first 10 N-terminal codons and last 10 C-terminal codons of ROR1. Alternatively, the antisense molecules are modified to employ ribozymes in the inhibition of ROR1 expression (L. A. Couture & D. T. Stinchcomb, 1996, Trends Genet. 12: 510-515).

Further specific embodiments of this aspect of the invention include primers and primer pairs, which allow the specific amplification of the polynucleotides of the invention or of any specific parts thereof, and probes that selectively or specifically hybridize to nucleic acid molecules of the invention or to any part thereof. Probes may be labeled with a detectable market, such as, for example, a radioisotope, fluorescent compound, bioluminescent compound, a chemiluminescent compound, metal chelator or enzyme. Such probes and primers can be used to detect the presence of a ROR1 polynucleotide in a sample and as a means for detecting a cell expressing a ROR1 protein.

Examples of such probes include polypeptides comprising all or part of the human ROR1 cDNA sequences shown in FIG. 1. Examples of primer pairs capable of specifically amplifying ROR1 mRNAs are easily made by those of skill in the art. As will be understood by the skilled artisan, a great many different primers and probes may be prepared based on the sequences provided herein and used effectively to amplify and/or detect a ROR1 mRNA.

As used herein, a polynucleotide is said to be “isolated” when it is substantially separated from contaminant polynucleotides that correspond or are complementary to genes other than the ROR1 gene or that encode polypeptides other than ROR1 gene product or fragments thereof. A skilled artisan can readily employ nucleic acid isolation procedures to obtain an isolated ROR1 polynucleotide.

The ROR1 polynucleotides of the invention are useful for a variety of purposes, including but not limited to their use as probes and primers for the amplification and/or detection of the ROR1 gene(s), mRNA(s), or fragments thereof; as reagents for the diagnosis and/or prognosis of breast cancer (e.g. specific breast cancer subtypes) and other cancers; as coding sequences capable of directing the expression of ROR1 polypeptides; as tools for modulating or inhibiting the expression of the ROR1 gene(s) and/or translation of the ROR1 transcript(s); and as therapeutic agents.

### Isolation of ROR1-Encoding Nucleic Acid Molecules

The ROR1 cDNA sequences described herein enable the isolation of other polynucleotides encoding ROR1 gene product(s), as well as the isolation of polynucleotides encoding ROR1 gene product homologs, alternatively spliced isoforms, allelic variants, and mutant forms of the ROR1 gene product. Various molecular cloning methods that can be employed to isolate full length cDNAs encoding a ROR1 gene are well known (See, e.g., Sambrook, J. et al., 1989, Molecular Cloning: A Laboratory Manual, 2d ed., Cold Spring Harbor Press, New York; Ausubel et al., eds., 1995, Current Protocols in Molecular Biology, Wiley and Sons). For example, lambda phage cloning methodologies may be conveniently employed, using commercially available cloning systems (e.g., Lambda ZAP Express, Stratagene). Phage clones containing ROR1 gene cDNAs may be identified by probing with a labeled ROR1 cDNA or a fragment thereof. For example, in one embodiment, the ROR1 cDNA (FIG. 1) or a portion thereof can be synthesized and used as a probe to retrieve overlapping and full length cDNAs corresponding to a ROR1 gene. The ROR1 gene itself may be isolated by screening genomic DNA libraries, bacterial artificial chromosome libraries (BACs), yeast artificial chromosome libraries (YACs), and the like, with ROR1 DNA probes or primers.

### Recombinant DNA Molecules and Host-Vector Systems

The invention also provides recombinant DNA or RNA molecules containing a ROR1 polynucleotide, including but not limited to phages, plasmids, phagemids, cosmids, YACs, BACs, as well as various vital and non-vital vectors well known in the art, and cells transformed or transfected with such recombinant DNA or RNA molecules. As used herein, a recombinant DNA or RNA molecule is a DNA or RNA molecule that has been subjected to molecular manipulation in vitro. Methods for generating such molecules are well known (see, e.g., Sambrook et al, 1989, supra).

The invention further provides a host-vector system comprising a recombinant DNA molecule containing a ROR1 polynucleotide within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 or HighFive cell). Examples of suitable mammalian cells include various breast cancer cell lines such as MDA 231, MCF-7, other transfectable or transducible breast cancer cell lines, as well as a number of mammalian cells routinely used for the expression of recombinant proteins (e.g., COS, CHO, MCF-7 cells). More particularly, a polynucleotide comprising the coding sequence of ROR1 may be used to generate ROR1 proteins or fragments thereof using any number of host-vector systems routinely used and widely known in the art.

A wide range of host-vector systems suitable for the expression of ROR1 proteins or fragments thereof are available (see, e.g., Sambrook et al., 1989, supra; Current Protocols in Molecular Biology, 1995, supra). Common vectors for mammalian expression include but are not limited to pcDNA 3.1 myc-His-tag (Invitrogen) and the retroviral vector pSRαtkneo (Muller et al., 1991, MCB 11:1785). Using these expression vectors, ROR1 may be preferably expressed in several breast cancer and non-breast cell lines, including for example, MCF-7, rat-1, NIH 3T3 and TsuPr1. The host-vector systems of the invention are useful for the production of a ROR1 protein or fragment thereof. Such host-vector systems may be employed to study the functional properties of ROR1 and ROR1 mutations.

Recombinant human ROR1 protein may be produced by mammalian cells transfected with a construct encoding ROR1. In an illustrative embodiment described in the Examples, MCF-7 cells can be transfected with an expression plasmid encoding ROR1, the ROR1 protein is expressed in the MCF-7 cells, and the recombinant ROR1 protein can be isolated using standard purification methods (e.g., affinity purification using anti-ROR1 antibodies). In another embodiment, also described in the Examples herein, the ROR1 coding sequence is subcloned into the retroviral vector pSRαMSVtkneo and used to infect various mammalian cell lines, such as NIH 3T3, MCF-7 and rat-1 in order to establish ROR1 expressing cell lines. Various other expression systems well known in the art may also be employed. Expression constructs encoding a leader peptide joined in frame to the ROR1 coding sequence may be used for the generation of a secreted form of recombinant ROR1 protein.

Proteins encoded by the ROR1 genes, or by fragments thereof, will have a variety of uses, including but not limited to generating antibodies and in methods for identifying ligands and other agents and cellular constituents that bind to a ROR1 gene product. Antibodies raised against a ROR1 protein or fragment thereof may be useful in diagnostic and prognostic assays, and imaging methodologies in the management of human cancers characterized by expression of ROR1 protein, including but not limited to cancers of the breast. Such antibodies may be expressed intracellularly and used in methods of treating patients with such cancers. Various immunological assays useful for the detection of ROR1 proteins are contemplated, including but not limited to various types of radioimmunoassays, enzyme-linked immunosorbent assays (ELISA), enzyme-linked immunofluorescent assays (ELIFA), immunocytochemical methods, and the like. Such antibodies may be labeled and used as immunological imaging reagents capable of detecting ROR1 expressing cells (e.g., in radioscintigraphic imaging methods). ROR1 proteins may also be particularly useful in generating cancer vaccines, as further described below.

### ROR1 Polypeptides

Another aspect of the present invention provides ROR1 proteins and polypeptide fragments thereof. The ROR1 proteins of the invention include those specifically identified herein, as well as allelic variants, conservative substitution variants and homologs that can be isolated/generated and characterized without undue experimentation following the methods outlined below. Fusion proteins that combine parts of different ROR1 proteins or fragments thereof, as well as fusion proteins of a ROR1 protein and a heterologous polypeptide are also included. Such ROR1 proteins will be collectively referred to as the ROR1 proteins, the proteins of the invention, or ROR1. As used herein, the term “ROR1 polypeptide” refers to a polypeptide fragment or a ROR1 protein of at least 6 amino acids, preferably at least 15 amino acids.

Specific embodiments of ROR1 proteins comprise a polypeptide having the amino acid sequence of human ROR1 as shown in FIG. 1. Alternatively, embodiments of ROR1 proteins comprise variant polypeptides having alterations in the amino acid sequence of human ROR1 as shown in FIG. 1.

In general, naturally occurring allelic variants of human ROR1 will share a high degree of structural identity and homology (e.g., 90% or more identity). Typically, allelic variants of the ROR1 proteins will contain conservative amino add substitutions within the ROR1 sequences described herein or will contain a substitution of an amino add from a corresponding position in a ROR1 homologue. One class of ROR1 allelic variants will be proteins that share a high degree of homology with at least a small region of a particular ROR1 amino acid sequence, but will further contain a radical departure from the sequence, such as a non-conservative substitution, truncation, insertion or frame shift.

Conservative amino acid substitutions can frequently be made in a protein without altering either the conformation or the function of the protein. Such changes include substituting any of isoleucine (9, valine (V), and leucine CL) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pips of these two amino acid residues are not significant. Still other changes can be considered “conservative” in particular environments.

Embodiments of the invention disclosed herein include a wide variety of art accepted variants of ROR1 proteins such as polypeptides having amino acid insertions, deletions and substitutions. ROR1 variants can be made using methods known in the art such as site-directed mutagenesis, alanine scanning, and PCR mutagenesis. Site-directed mutagenesis (Carter et al., 1986, Nucl. Acids Res. 13:4331; Zoller et al., 1987, Nucl. Acids Res. 10:6487), cassette mutagenesis (Wells et al., 1985, Gene 34:315), restriction selection mutagenesis (Wells et al., 1986, Philos. Trans. R. Soc. London Set. A, 317:415) or other known techniques can be performed on the cloned DNA to produce the ROR1 variant DNA. Scanning amino acid analysis can also be employed to identify one or more amino acids along a contiguous sequence. Among the common scanning amino acids are relatively small, neutral amino acids. Such amino acids include alanine, glycine, serine, and cysteine. Alanine is typically a common scanning amino acid among this group because it eliminates the side-chain beyond the beta-carbon and is less likely to alter the main-chain conformation of the variant. Alanine is also typically used because it is the most common amino acid. Further, it is frequently found in both buried and exposed positions (Creighton, The Proteins, (W. H. Freeman & Co., N.Y.); Chothia, 1976, J. Mol. Biol., 150:1). If alanine substitution does not yield adequate amounts of variant, an isosteric amino acid can be used.

As discussed above, embodiments of the claimed invention include polypeptides containing less than the 937 amino acid sequence of the ROR1 protein shown in FIG. 1 (and the polynucleotides encoding such polypeptides). For example, representative embodiments of the invention disclosed herein include polypeptides consisting of about amino acid 1 to about amino acid 10 of the ROR1 protein shown in FIG. 1, polypeptides consisting of about amino acid 20 to about amino acid 30 of the ROR1 protein shown in FIG. 1, polypeptides consisting of about amino acid 30 to about amino acid 40 of the ROR1 protein shown in FIG. 1, polypeptides consisting of about amino acid 40 to about amino acid 50 of the ROR1 protein shown in FIG. 1, polypeptides consisting of about amino acid 50 to about amino acid 60 of the ROR1 protein shown in FIG. 1, polypeptides consisting of about amino acid 60 to about amino acid 70 of the ROR1 protein shown in FIG. 1, polypeptides consisting of about amino acid 70 to about amino acid 80 of the ROR1 protein shown in FIG. 1, polypeptides consisting of about amino acid 80 to about amino acid 90 of the ROR1 protein shown in FIG. 1 and polypeptides consisting of about amino acid 90 to about amino acid 100 of the ROR1 protein show in FIG. 1, etc. Following this scheme, polypeptides consisting of portions of the amino acid sequence of amino acids 100-937 of the ROR1 protein are typical embodiments of the invention. Polypeptides consisting of larger portions of the ROR1 protein are also contemplated. For example polypeptides consisting of about amino acid 1 (or 20 or 30 or 40 etc.) to about amino acid 20, (or 30, or 40 or 50 etc.) of the ROR1 protein show in FIG. 1 may be generated by a variety of techniques well known in the art.

The polypeptides of the preceding paragraphs have a number of different specific uses. As ROR1 is shown to be highly expressed in certain breast cancer subtypes as compared to corresponding normal breast tissue, these polypeptides may be used in methods assessing the status of ROR1 gene products in normal versus cancerous tissues and elucidating the malignant phenotype. Typically, polypeptides encoding specific regions of the ROR1 protein may be used to assess the presence of perturbations (such as deletions, insertions, point mutations etc.) in specific regions of the ROR1 genre products. Exemplary assays can utilize antibodies targeting a ROR1 polypeptide containing the amino acid residues of one or more of the biological motifs contained within the ROR1 polypeptide sequence in order to evaluate the characteristics of this region in normal versus cancerous tissues. Alternatively, ROR1 polypeptides containing the amino acid residues of one or more of the biological motifs contained within the ROR1 polypeptide sequence can be used to screen for factors that interact with that region of ROR1.

As discussed above, redundancy in the genetic code permits variation in ROR1 gene sequences. In particular, one skilled in the art will recognize specific codon preferences by a specific host species and can adapt the disclosed sequence as preferred for a desired host. For example, certain codon sequences typically have tare codons (i.e., codons having a usage frequency of less than about 20% in known sequences of the desired host) replaced with higher frequency codons. Codon preferences for a specific organism may be calculated, for example, by utilizing codon usage tables available on the Internet at the following address: www.dna.affrc.go.jp/˜nakamura/codon.html. Nucleotide sequences that have been optimized for a particular host species by replacing any codons having a usage frequency of less than about 20% are referred to herein as “codon optimized sequences.”

Additional sequence modifications are known to enhance protein expression in a cellular host. These include elimination of sequences encoding spurious polyadenylation signals, exon/intron splice site signals, transposon-like repeats, and/or other such well-characterized sequences that may be deleterious to gene expression. The GC content of the sequence may be adjusted to levels average for a given cellular host, as calculated by reference to known genes expressed in the host cell. Where possible, the sequence may also be modified to avoid predicted hairpin secondary mRNA structures. Other useful modifications include the addition of a translational initiation consensus sequence at the start of the open reading frame, as described in Kozak, 1989, Mol. Cell. Biol., 9:5073-5080. Nucleotide sequences that have been optimized for expression in a given host species by elimination of spurious polyadenylation sequences, elimination of exon/intron splicing signals, elimination of teaspoon-like repeats and/or optimization of GC content in addition to codon optimization are referred to herein as an “expression enhanced sequence.”

ROR1 proteins may be embodied in many forms, preferably in isolated form. As used herein, a protein is said to be “isolated” when physical, mechanical or chemical methods are employed to remove the ROR1 protein from cellular constituents that are normally associated with the protein. A skilled artisan can readily employ standard purification methods to obtain an isolated ROR1 protein. A purified ROR1 protein molecule will be substantially free of other proteins or molecules that impair the binding of ROR1 to antibody or other ligand. The nature and degree of isolation and purification will depend on the intended use. Embodiments of a ROR1 protein include a purified ROR1 protein and a functional, soluble ROR1 protein. In one form, such functional, soluble ROR1 proteins or fragments thereof retain the ability to bind antibody or other ligand.

The invention also provides ROR1 polypeptides comprising biologically active fragments of the ROR1 amino acid sequence, such as a polypeptide corresponding to part of the amino acid sequence for ROR1 as shown in FIG. 1. Such polypeptides of the invention exhibit properties of the ROR1 protein, such as the ability to elicit the generation of antibodies that specifically bind an epitope associated with the ROR1 protein.

ROR1 polypeptides can be generated using standard peptide synthesis technology or using chemical cleavage methods well known in the art based on the amino add sequences of the human ROR1 proteins disclosed herein. Alternatively, recombinant methods can be used to generate nucleic acid molecules that encode a polypeptide fragment of a ROR1 protein. In this regard, the ROR1-encoding nucleic acid molecules described herein provide means for generating defined fragments of ROR1 proteins. ROR1 polypeptides are particularly useful in generating and characterizing domain specific antibodies (e.g., antibodies recognizing an extracellular or intracellular epitope of a ROR1 protein), in identifying agents or cellular factors that bind to ROR1 or a particular structural domain thereof, and in various therapeutic contexts, including but not limited to cancer vaccines.

ROR1 polypeptides containing particularly interesting structures can be predicted and/or identified using various analytical techniques well known in the art, including, for example, the methods of Chou-Fasman, Garnier-Robson, Kyte-Doolittle, Eisenberg, Karplus-Schultz or Jameson-Wolf analysis, or on the basis of immunogenicity. Fragments containing such structures are particularly useful in generating subunit specific anti-ROR1 antibodies or in identifying cellular factors that bind to ROR1.

In an embodiment described in the examples that follow, ROR1 can be conveniently expressed in cells (such as MCF-7 cells) transfected with a commercially available expression vector such as a CMV-driven expression vector encoding ROR1 with a C-terminal 6XHis and MYC tag (pcDNA3.1/mycHIS, Invitrogen or Tag5, GenHunter Corporation, Nashville Tenn.). The Tag5 vector provides an IgGK secretion signal that can be used to facilitate the production of a secreted ROR1 protein in transfected cells. The secreted HIS-tagged ROR1 in the culture media may be purified using a nickel column using standard techniques.

The ROR1 of the present invention may also be modified in a way to form a chimeric molecule comprising ROR1 fused to another, heterologous polypeptide or amino acid sequence. In one embodiment, such a chimeric molecule comprises a fusion of the ROR1 with a polyhistidine epitope tag, which provides an epitope to which immobilized nickel can selectively bind. The epitope tag is generally placed at the amino- or carboxyl-terminus of the ROR1. In an alternative embodiment, the chimeric molecule may comprise a fusion of the ROR1 with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a ROR1 polypeptide in place of at least one variable region within an Ig molecule. In particular embodiments, the immunoglobulin fusion includes the hinge, CH₂ and CH₃, or the hinge, CH1, CH2 and CH3 regions of an IgG1 molecule. For the production of immunoglobulin fusions see also U.S. Pat. No. 5,428,130 issued Jun. 27, 1995.

In some embodiments of the invention, the fusion protein includes only the Ig-like C2-type domain of ROR1 (Q73-V139 of SEQ ID NO: 2). In some embodiments of the invention, the fusion protein includes only the frizzled domain of ROR1 (E165-I299 of SEQ ID NO: 2). In some embodiments of the invention, the fusion protein includes only the kringle domain of ROR1 (K312-C391 of SEQ ID NO: 2). In other embodiments of the invention, the fusion protein includes 2 or alternatively 3 of these ROR1 domains.

### ROR1 Antibodies

The term “antibody” is used in the broadest sense and specifically covers single anti-ROR1 monoclonal antibodies (including agonist, antagonist and neutralizing antibodies) and anti-ROR1 antibody compositions with polyepitopic specificity. The term “monoclonal antibody” (mAb) as used herein refers to an antibody obtained from a population of substantially homogeneous antibodies, i.e. the antibodies comprising the individual population are identical except for possible naturally-occurring mutations that may be present in minor amounts.

Another aspect of the invention provides antibodies that bind to ROR1 proteins and polypeptides. The most common antibodies will specifically bind to a ROR1 protein and will not bind (or will bind weakly) to non-ROR1 proteins and polypeptides. Anti-ROR1 antibodies that are particularly contemplated include monoclonal and polyclonal antibodies as well as fragments containing the antigen binding domain and/or one or more complementarity determining regions of these antibodies. As used herein, an antibody fragment is defined as at least a portion of the variable region of the immunoglobulin molecule that binds to its target, i.e., the antigen binding region.

ROR1 antibodies of the invention may be particularly useful in breast cancer diagnostic and prognostic assays, and imaging methodologies. Intracellularly expressed antibodies (e.g., single chain antibodies) may be therapeutically useful in treating cancers in which the expression of ROR1 is involved, such as for example advanced and metastatic breast cancers. Such antibodies may be useful in the treatment, diagnosis, and/or prognosis of other cancers, to the extent ROR1 is also expressed or overexpressed in other types of cancers such as breast cancers.

The invention also provides various immunological assays useful for the detection and quantification of ROR1 and mutant ROR1 proteins and polypeptides. Such assays generally comprise one or more ROR1 antibodies capable of recognizing and binding a ROR1 or mutant ROR1 protein, as appropriate, and may be performed within various immunological assay formats well known in the art, including but not limited to various types of radioimmunoassays, enzyme-linked immunosorbent assays ELISA), enzyme-linked immunofluorescent assays (ELIFA), and the like. In addition, immunological imaging methods capable of detecting breast cancer and other cancers expressing ROR1 are also provided by the invention, including but limited to radioscintigraphic imaging methods using labeled ROR1 antibodies. Such assays may be clinically useful in the detection, monitoring, and prognosis of ROR1 expressing cancers such as breast cancer.

ROR1 antibodies may also be used in methods for purifying ROR1 and mutant ROR1 proteins and polypeptides and for isolating ROR1 homologues and related molecules. For example, in one embodiment, the method of purifying a ROR1 protein comprises incubating a ROR1 antibody, which has been coupled to a solid matrix, with a lysate or other solution containing ROR1 under conditions that permit the ROR1 antibody to bind to ROR1; washing the solid matrix to eliminate impurities; and eluting the ROR1 from the coupled antibody. Other uses of the ROR1 antibodies of the invention include generating anti-idiotypic antibodies that mimic the ROR1 protein.

Various methods for the preparation of antibodies are well known in the art. For example, antibodies may be prepared by immunizing a suitable mammalian host using a ROR1 protein, peptide, or fragment, in isolated or immunoconjugated form (Harlow, and Lane, eds., 1988, Antibodies: A Laboratory Manual, CSH Press; Harlow, 1989, Antibodies, Cold Spring Harbor Press, NY). In addition, fusion proteins of ROR1 may also be used, such as a ROR1GST-fusion protein. In a particular embodiment, a GST fusion protein comprising all ort most of the open reading frame amino acid sequence of FIG. 1 may be produced and used as an immunogen to generate appropriate antibodies. In another embodiment, a ROR1 peptide may be synthesized and used as an immunogen.

In addition, naked DNA immunization techniques known in the art may be used (with or without purified ROR1 protein or ROR1 expressing cells) to generate an immune response to the encoded immunogen (for review, see Donnelly et al., 1997, Ann. Rev. Immunol. 15:617-648).

The amino acid sequence of the ROR1 as shown in FIG. 1 may be used to select specific regions of the ROR1 protein for generating antibodies. For example, hydrophobicity and hydrophilicity analyses of the ROR1 amino acid sequence may be used to identify hydrophilic regions in the ROR1 structure. Regions of the ROR1 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis.

Methods for preparing a protein or polypeptide for use as an immunogen and for preparing immunogenic conjugates of a protein with a carrier such as BSA, KLH, or other carrier proteins are well known in the art. In some circumstances, direct conjugation using, for example, carbodiimide reagents may be used; in other instances linking reagents such as those supplied by Pierce Chemical Co., Rockford, Ill., may be effective. Administration of a ROR1 immunogen is conducted generally by injection over a suitable time period and with use of a suitable adjuvant, as is generally understood in the art. During the immunization schedule, titers of antibodies can be taken to determine adequacy of antibody formation.

ROR1 monoclonal antibodies may be produced by various means well known in the art. For example, immortalized cell lines that secrete a desired monoclonal antibody may be prepared using the standard hybridoma technology of Kohler and Milstein or modifications that immortalize producing B cells, as is generally known. The immortalized cell lines secreting the desired antibodies are screened by immunoassay in which the antigen is the ROR1 protein or a ROR1 fragment. When the appropriate immortalized cell culture secreting the desired antibody is identified, the cells may be expanded and antibodies produced either from in vitro cultures or from ascites fluid.

The antibodies or fragments may also be produced, using current technology, by recombinant means. Regions that bind specifically to the desired regions of the ROR1 protein can also be produced in the context of chimeric or CDR grafted antibodies of multiple species origin. Humanized or human ROR1 antibodies may also be produced for use in therapeutic contexts. Methods for humanizing murine and other non-human antibodies by substituting one or more of the non-human antibody CDRs for corresponding human antibody sequences are well known (see for example, Jones et al., 1986, Nature 321:522-525; Riechmann et al., 1988, Nature 332:323-327; Verhoeyen et al., 1988, Science 239:1534-1536). See also, Carter et al., 1993, Proc. Natl. Acad. Sci. USA 89:4285 and Sims et al., 1993, J. Immunol. 151:2296. Methods for producing fully human monoclonal antibodies include phage display and transgenic methods (for review, see Vaughan et al., 1998, Nature Biotechnology 16:535-539).

Fully human ROR1 monoclonal antibodies may be generated using cloning technologies employing large human Ig gene combinatorial libraries (i.e., phage display) (Griffiths and Hoogenboom, Building an in vitro immune system: human antibodies from phage display libraries. In: Clark, M., ed., 1993, Protein Engineering of Antibody Molecules for Prophylactic and Therapeutic Applications in Man, Nottingham Academic, pp 45-64; Burton and Barbas, Human Antibodies from combinatorial libraries. Id., pp 65-82). Fully human ROR1 monoclonal antibodies may also be produced using transgenic mice engineered to contain human immunoglobulin gene loci as described in PCT Patent Application WO98/24893, Kucherlapati and Jakobovits et al., published Dec. 3, 1997 (see also, Jakobovits, 1998, Exp. Opin. Invest. Drugs 7(4):607-614). This method avoids the in vitro manipulation required with phage display technology and efficiently produces high affinity authentic human antibodies.

Reactivity of ROR1 antibodies with a ROR1 protein may be established by a number of well known means, including western blot, immunoprecipitation, ELISA, and FACS analyses using, as appropriate, ROR1 proteins, peptides, ROR1-expressing cells or extracts thereof.

A ROR1 antibody or fragment thereof of the invention may be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. A second molecule for conjugation to the ROR1 antibody can be selected in accordance with the intended use. For example, for therapeutic use, the second molecule can be a toxin or therapeutic agent. Further, bi-specific antibodies specific for two or more ROR1 epitopes may be generated using methods generally known in the art. Homodimeric antibodies may also be generated by cross-linking techniques known in the art (e.g., Wolff et al., 1993, Cancer Res. 53: 2560-2565).

An illustrative embodiment of the invention is an isolated antibody which specifically binds to an ROR1 polypeptide sequence shown in FIG. 1 (SEQ ID NO: 2). Optionally this isolated antibody specifically binds to the extracellular region of ROR1 (M1-V406 of SEQ ID NO: 2). In certain embodiments of the invention, the isolated antibody specifically binds to the Ig-like C2-type domain of ROR1 (Q73-V139 of SEQ ID NO: 2). In other embodiments of the invention, the isolated antibody specifically binds to the frizzled domain of ROR1 (E165-1299 of SEQ ID NO: 2). In other embodiments of the invention, the isolated antibody specifically binds to the kringle domain of ROR1 (K312-C391 of SEQ ID NO: 2).

Another embodiment of the invention is an immunotoxin which is a conjugate of a cytotoxic moiety and one of these antibodies. Optionally, the antibody is an antibody fragment comprising an antigen binding region which specifically binds to ROR1 (e.g. a Fab fragment). Typically one or more of these antibodies will down regulates the ROR1 and/or is capable of activating complement in a patient treated with an effective amount of the antibodies and/or is capable of mediating antibody dependent cellular cytotoxicity in a patient treated with an effective amount of the antibody. In certain embodiments of the invention, one or more of these antibodies eliminates and/or reduces tumor burden in a patient treated with an effective amount of the antibody. In certain embodiments of the invention, the tumor cell is a human breast carcinomas of the BRCA1 and/or basal subtype. Another related embodiment of the invention is a hybridoma that produces one of these antibodies which specifically binds to ROR1. Another related embodiment of the invention is a composition comprising one of these antibodies which specifically binds to ROR1 and a pharmaceutically acceptable carrier. Yet another embodiment of the invention is an assay for detecting a tumor (e.g. a breast cancer) comprising the steps of exposing a cell to one of these antibodies and then determining the extent of binding of the antibody to the cell.

A related embodiment of the invention is an antibody which specifically binds to the extracellular domain of the ROR1 and inhibits growth of tumor cells which overexpress ROR1 in a patient treated with an effective amount of the antibody. In certain embodiments of the invention, the tumor cell is a human breast carcinomas of the BRCA1 and/or basal subtype. Optionally the antibody is a murine monoclonal antibody. Typically the antibody down regulates the ROR1 and/or is capable of activating complement in a patient and/or is capable of mediating antibody dependent cellular cytotoxicity in the patient. A related embodiment of the invention is an immunotoxin which is a conjugate of a cytotoxic moiety and this antibody. Another related embodiment of the invention is a hybridoma producing this antibody.

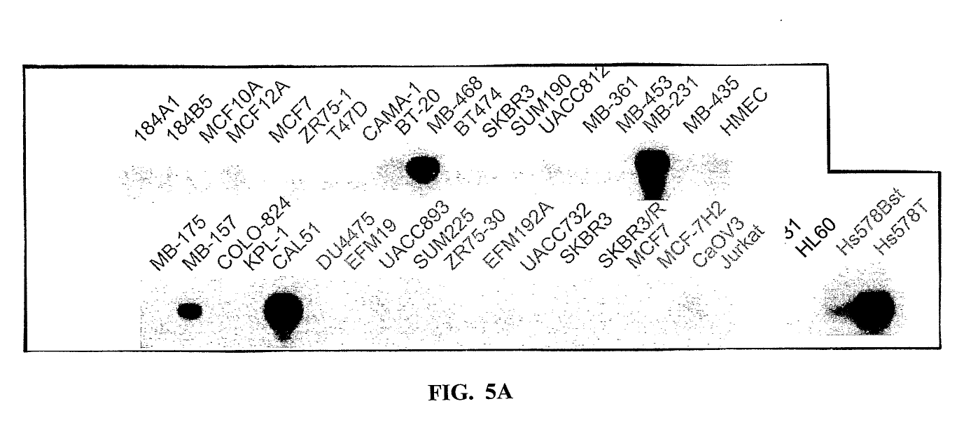

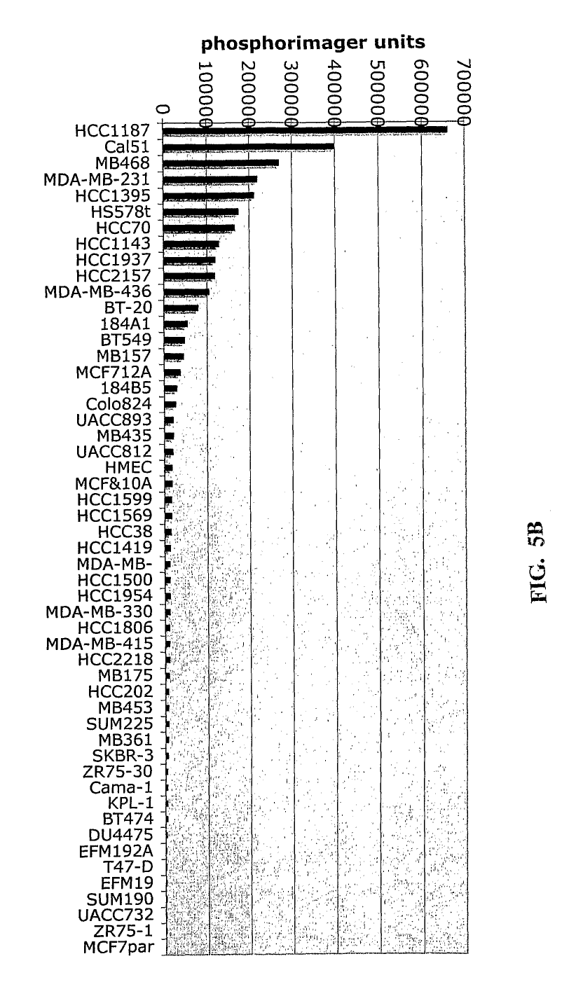

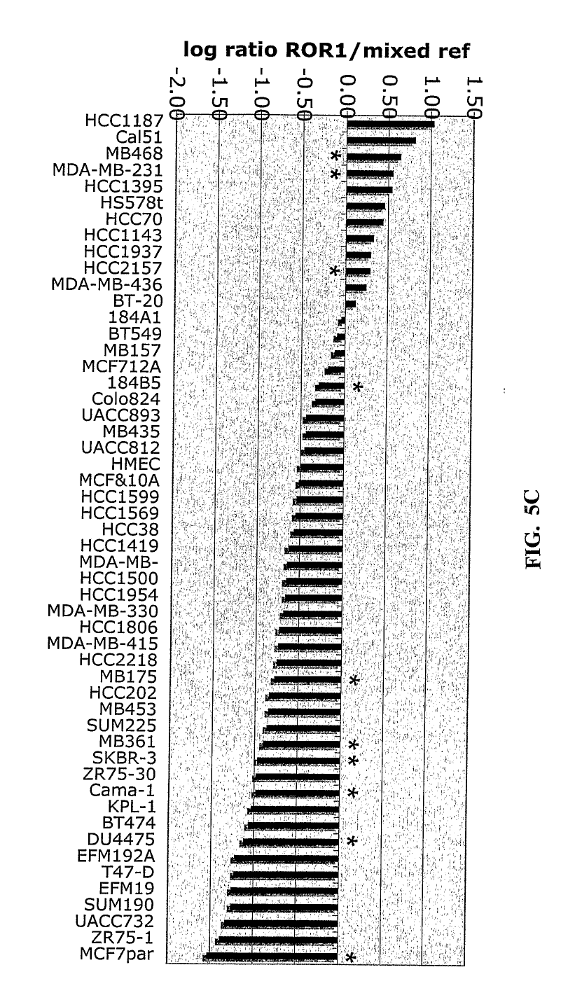

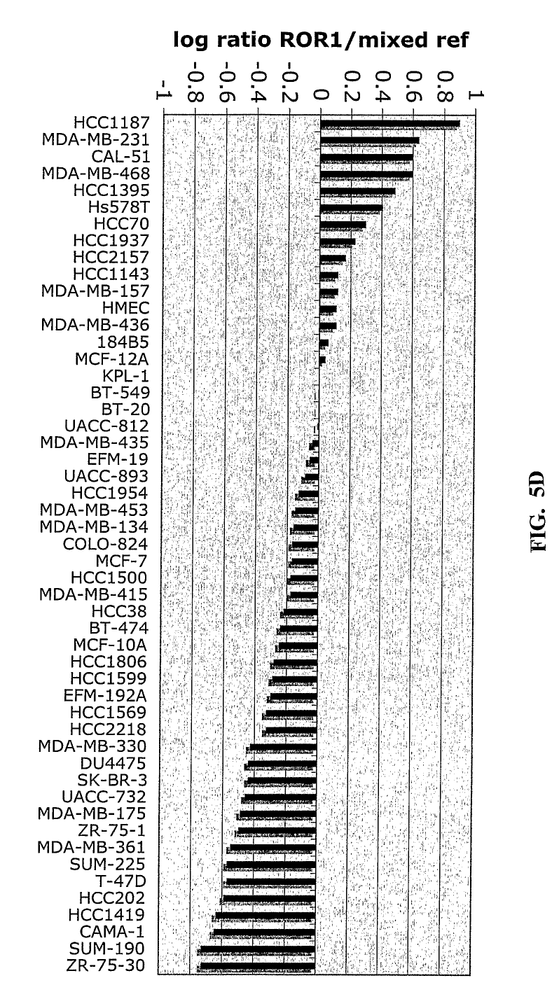

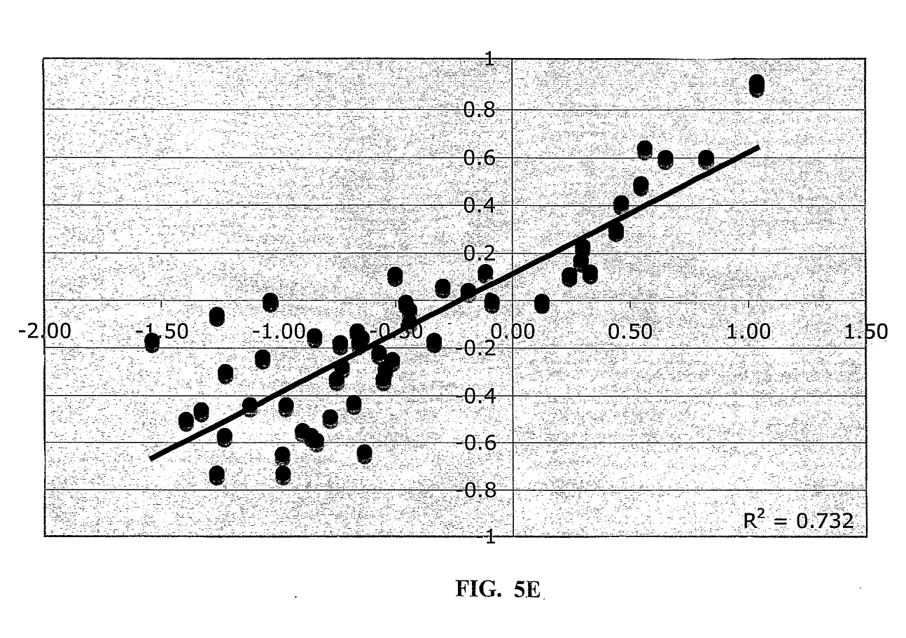

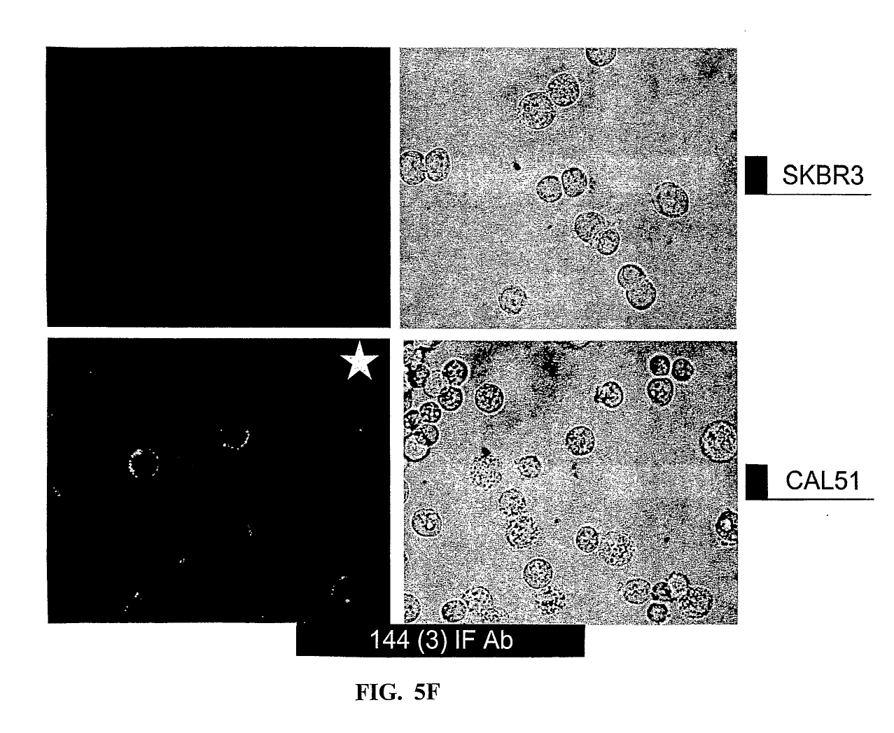

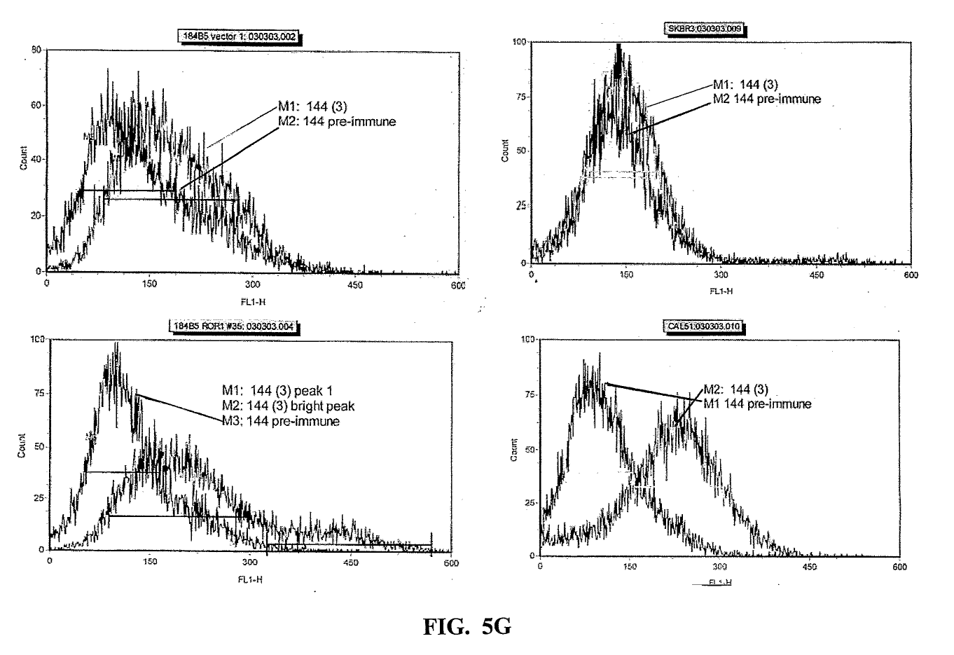

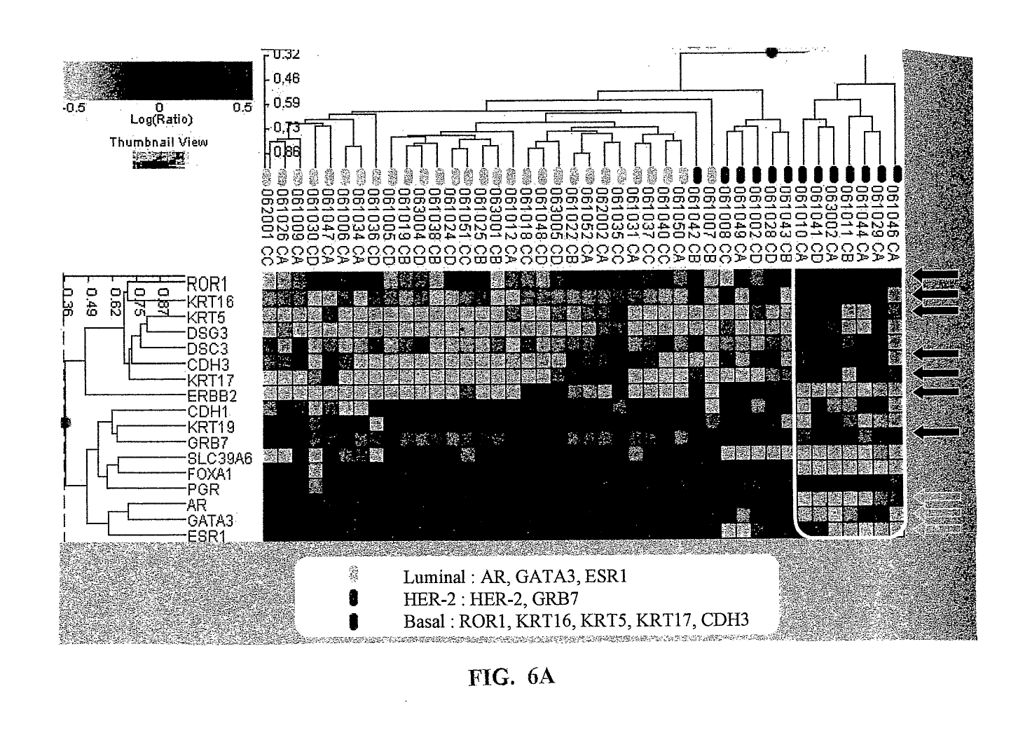

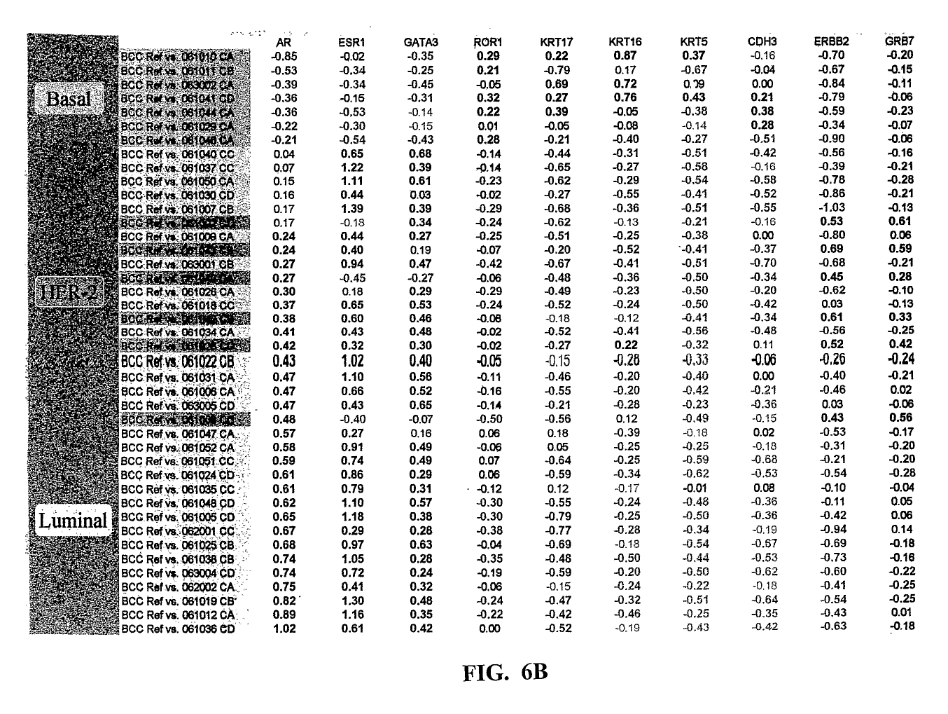

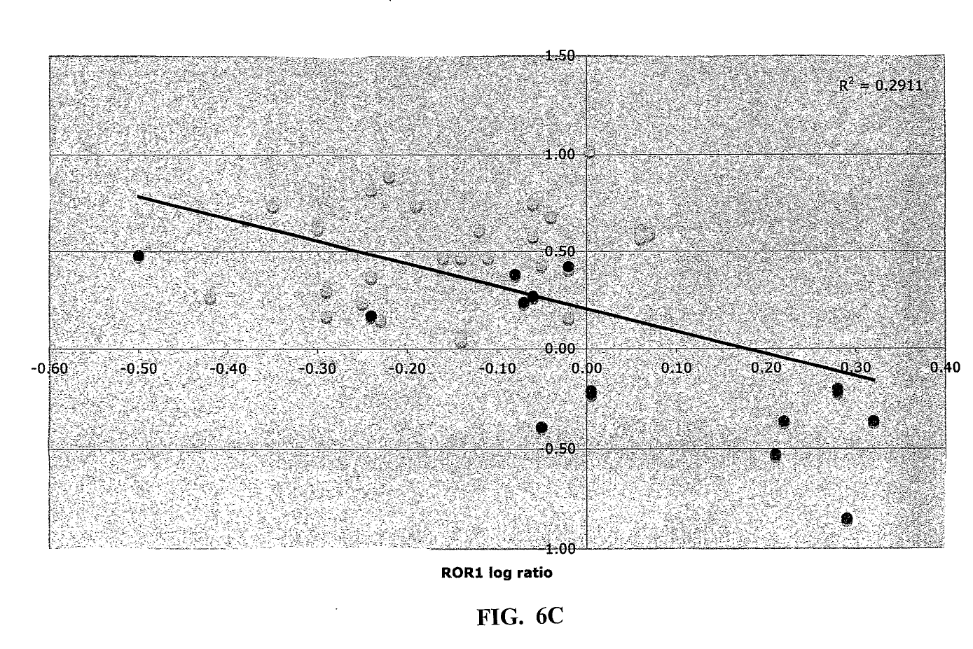

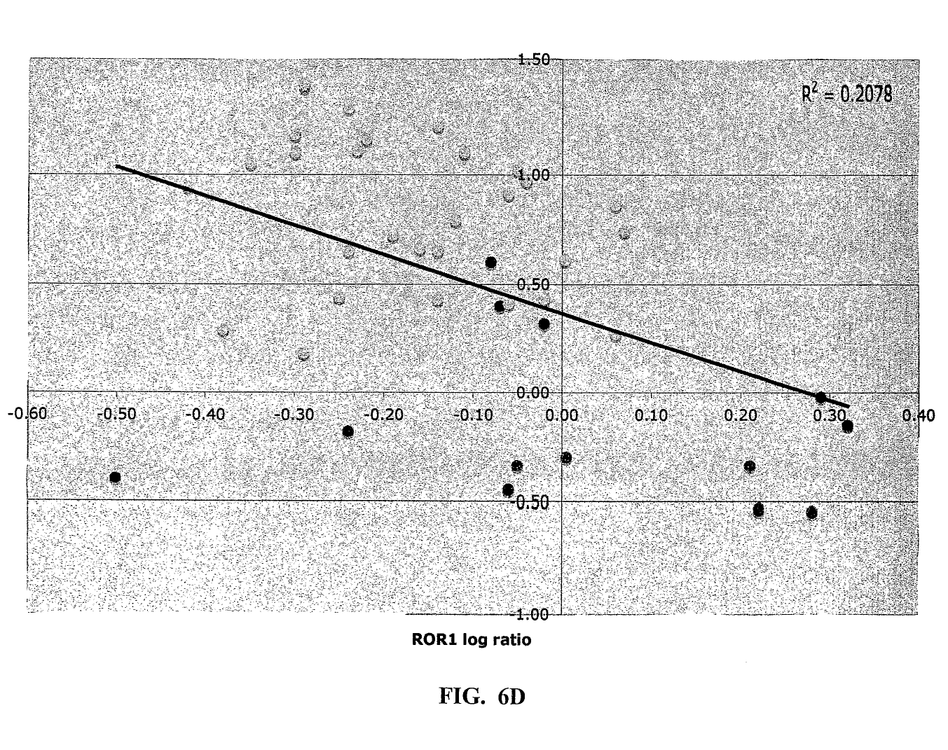

Another embodiment of the invention is an antibody which specifically binds to ROR1 and inhibits the growth of HCC1187, Cal51, MB468, MDA-MB-231, HCC1395, HS578T, HCC70, HCC1143, HCC1937, HCC2157, MDA-MB-436, BT-20, 184A1, MB157, MCF12A, 184B5, or Colo824 tumor cells (see, e.g. FIG. 5) in cell culture by greater than 20%, at an antibody concentration of about 0.5, 1, 5, 10, or 30 μg/ml. Typically these tumor cells are cultured in culture medium comprising 10% fetal bovine serum and the growth inhibition is determined approximately six days after exposure of the tumor cells to the antibody. Typically this antibody is a monoclonal antibody. Optionally this monoclonal antibody binds to the extracellular region of ROR1 (M1-V406 or Q30-V406 of SEQ ID NO: 2). In certain embodiments of the invention, the monoclonal antibody binds to the Ig-like C2-type domain of ROR1 (Q73-V139 of SEQ ID NO: 2). In other embodiments of the invention, the monoclonal antibody binds to the frizzled domain of ROR1 (E165-I299 of SEQ ID NO: 2). In other embodiments of the invention, the monoclonal antibody binds to the kringle domain of ROR1 (K312-C391 of SEQ ID NO: 2). In some embodiments of the invention, this antibody downregulates ROR1 on a tumor cell that overexpresses this polypeptide and inhibits growth of tumor cells in a patient treated with a therapeutically effective amount of this antibody. In certain embodiments of the invention, the tumor cell is a human breast carcinomas of the BRCA1 and/or basal subtype. Typically the antibody is capable of activating complement in a patient and/or is capable of mediating antibody dependent cellular cytotoxicity in the patient. A related embodiment of the invention is an immunotoxin which is a conjugate of a cytotoxic moiety and this antibody. Another related embodiment of the invention is a hybridoma producing this antibody.

Yet another embodiment of the invention is a method of inhibiting the growth of tumor cells that overexpress ROR1 comprising administering to a patient an antibody which binds specifically to the extracellular domain of the ROR1 in an amount effective to inhibit growth of the tumor cells in the patient. In certain embodiments of the invention, the tumor cell is a human breast carcinomas of the BRCA1 and/or basal subtype. Typically the antibody is capable of activating complement in a patient and/or is capable of mediating antibody dependent cellular cytotoxicity in the patient. A related embodiment of the invention is an immunotoxin which is a conjugate of a cytotoxic moiety and this antibody. Another related embodiment of the invention is a hybridoma producing this antibody.

Yet another embodiment of the invention is a method of inhibiting the growth of tumor cells that overexpress ROR1 comprising administering to a patient an antibody comprising an antigen binding region which specifically binds to an extracellular domain of the ROR1 in an amount effective to inhibit growth of the tumor cells in the patient, wherein the antibody is not conjugated to a cytotoxic moiety. In certain embodiments of the invention, the tumor cell is a human breast carcinomas of the BRCA1 and/or basal subtype. A related embodiment of the invention is a method of treating cancer that overexpresses ROR1 comprising administering to a patient an antibody comprising an antigen binding region which specifically binds to an extracellular domain of the ROR1 in an amount effective to eliminate or reduce the patient's tumor burden, wherein the antibody is not conjugated to a cytotoxic moiety. Optionally the patient has breast cancer. Yet another embodiment of the invention is a method of treating cancer comprising identifying a patient with cancer characterized by amplification of the HER2 gene and/or overexpression of the ROR1 and administering to the patient thus identified an antibody comprising an antigen binding region which specifically binds to an extracellular domain of the ROR1 in an amount effective to inhibit growth of the cancer of the patient.

Another embodiment of the invention is a method of treating a patient having a carcinoma that overexpresses ROR1 comprising administering to the patient an antibody which binds specifically to the extracellular domain of the ROR1 in an amount effective to eliminate or reduce the patient's tumor burden. In certain embodiments of the invention, the tumor cell is a human breast carcinomas of the BRCA1 and/or basal subtype. Typically this antibody is a monoclonal antibody. In some embodiments of the invention, this antibody downregulates the ROR1 on a tumor cell that overexpresses this polypeptide and inhibits growth of tumor cells in a patient treated with a therapeutically effective amount of this antibody. Typically the antibody is capable of activating complement in a patient and/or is capable of mediating antibody dependent cellular cytotoxicity in the patient. A related embodiment of the invention is an immunotoxin which is a conjugate of a cytotoxic moiety and this antibody. Another related embodiment of the invention is a hybridoma producing this antibody.

Other related embodiments of the invention include methods for the preparation of a medication for the treatment of pathological conditions including breast cancer by preparing an anti-ROR1 antibody composition for administration to a mammal having the pathological condition. A related method is the use of an effective amount of an anti-ROR1 antibody in the preparation of a medicament for the treatment of a breast cancer. Another related method is the use of an effective amount of an anti-ROR1 antibody in the preparation of a medicament for the treatment of a basal breast cancer. A related method is the use of an effective amount of an anti-ROR1 antibody in the preparation of a medicament for the treatment of a BRCA1 breast cancer. Yet another related embodiment is a use of a anti-ROR1 antibody the manufacture of a medicament for inhibiting ROR1 action in a patient. Such methods typically involve the steps of including an amount of anti-ROR1 antibody sufficient to inhibit ROR1 signaling in vivo and an appropriate amount of a physiologically acceptable carrier. As is known in the art, optionally other agents can be included in these preparations.

### ROR1 Transgenic Animals

Nucleic acids that encode ROR1 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA that is integrated into the genome of a cell from which a transgenic animal develops. In one embodiment, cDNA encoding ROR1 can be used to clone genomic DNA encoding ROR1 in accordance with established techniques and the genomic sequences used to generate transgenic animals that contain cells that express DNA encoding ROR1. Methods for generating transgenic animals, particularly animals such as mice or tats, have become conventional in the art and are described, for example, in U.S. Pat. Nos. 4,736,866 and 4,870,009. Typically, particular cells would be targeted for ROR1 transgene incorporation with tissue-specific enhancers. Transgenic animals that include a copy of a transgene encoding ROR1 introduced into the germ line of the animal at an embryonic stage can be used to examine the effect of increased expression of DNA encoding ROR1. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals beating the transgene, would indicate a potential therapeutic intervention for the pathological condition.

Alternatively, non-human homologues of ROR1 can be used to construct a ROR1 “knock out” animal that has a defective or altered gene encoding ROR1 as a result of homologous recombination between the endogenous gene encoding ROR1 and altered genomic DNA encoding ROR1 introduced into an embryonic cell of the animal. For example, cDNA encoding ROR1 can be used to clone genomic DNA encoding ROR1 in accordance with established techniques. A portion of the genomic DNA encoding ROR1 can be deleted or replaced with another gene, such as a gene encoding a selectable market that can be used to monitor integration. Typically, several kilobases of unaltered flanking DNA (both at the 5′ and 3′ ends) are included in the vector (see e.g., Thomas and Capecchi, 1987, Cell 51:503) for a description of homologous recombination vectors]. The vector is introduced into an embryonic stem cell line (e.g., by electroporation) and cells in which the introduced DNA has homologously recombined with the endogenous DNA are selected (see e.g., Li et al., 1992, Cell 69:915). The selected cells are then injected into a blastocyst of an animal (e.g., a mouse or rat) to form aggregation chimeras (see e.g., Bradley, in Robertson, ed., 1987, Teratocarcinomas and Embryonic Stem Cells: A Practical Approach, (IRL, Oxford), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the ROR1 polypeptide.

### Methods for the Detection of ROR1

Another aspect of the present invention relates to methods for detecting ROR1 polynucleotides and ROR1 proteins and variants thereof, as well as methods for identifying a cell that expresses ROR1. The expression profile of ROR1 makes it a potential diagnostic market for breast cancer and breast cancer subtype. In this context, the status of ROR1 gene products may provide information useful for predicting a variety of factors including susceptibility to advanced stage disease, rate of progression, and/or tumor aggressiveness. As discussed in detail below, the status of ROR1 gene products in patient samples may be analyzed by a variety protocols that are well known in the art including immunohistochemical analysis, the variety of Northern blotting techniques including in situ hybridization, RT-PCR analysis (for example on laser capture micro-dissected samples), western blot analysis and tissue array analysis.

More particularly, the invention provides assays for the detection of ROR1 polynucleotides in a biological sample, such as a breast biopsy and the like. Detectable ROR1 polynucleotides include, for example, a ROR1 gene or fragments thereof, ROR1 mRNA, alternative splice variant ROR1 mRNAs, and recombinant DNA or RNA molecules containing a ROR1 polynucleotide. A number of methods for amplifying and/or detecting the presence of ROR1 polynucleotides are well known in the art and may be employed in the practice of this aspect of the invention.

In one embodiment, a method for detecting a ROR1mRNA in a biological sample comprises producing cDNA from the sample by reverse transcription using at least one primer; amplifying the cDNA so produced using a ROR1 polynucleotides as sense and antisense primers to amplify ROR1 cDNAs therein; and detecting the presence of the amplified ROR1 cDNA Optionally, the sequence of the amplified ROR1 cDNA can be determined. In another embodiment, a method of detecting a ROR1 gene in a biological sample comprises first isolating genomic DNA from the sample; amplifying the isolated genomic DNA using ROR1 polynucleotides as sense and antisense primers to amplify the ROR1 gene therein; and detecting the presence of the amplified ROR1 gene. Any number of appropriate sense and antisense probe combinations may be designed from the nucleotide sequences provided for the ROR1 (FIG. 1) and used for this purpose.

The invention also provides assays for detecting the presence of a ROR1 protein in a tissue of other biological sample such as breast cell preparations, and the like. Methods for detecting a ROR1 protein are also well known and include, for example, immunoprecipitation, immunohistochemical analysis, Western Blot analysis, molecular binding assays, ELISA, ELIFA and the like. For example, in one embodiment, a method of detecting the presence of a ROR1 protein in a biological sample comprises first contacting the sample with a ROR1 antibody, a ROR1-reactive fragment thereof, or a recombinant protein containing an antigen binding region of a ROR1 antibody; and then detecting the binding of ROR1 protein in the sample thereto.

In some embodiments of the invention, the expression of ROR1 proteins in a sample is examined using Immunohistochemical staining protocols. Immunohistochemical staining of tissue sections has been shown to be a reliable method of assessing alteration of proteins in a heterogeneous tissue. Immunohistochemistry (IHC) techniques utilize an antibody to probe and visualize cellular antigens in situ, generally by chromogenic or fluorescent methods. This technique excels because it avoids the unwanted effects of disaggregation and allows for evaluation of individual cells in the context of morphology. In addition, the target protein is not altered by the freezing process.

Certain protocols that examine the expression of ROR1 proteins in a sample typically involve the preparation of a tissue sample followed by immunohistochemistry. Illustrative protocols are provided below. For sample preparation, any tissue sample from a subject may be used. Examples of tissue samples that may be used include, but are not limited to breast tissue. The tissue sample can be obtained by a variety of procedures including, but not limited to surgical excision, aspiration or biopsy. The tissue may be fresh or frozen. In one embodiment, the tissue sample is fixed and embedded in paraffin or the like. The tissue sample may be fixed (i.e. preserved) by conventional methodology (See e.g., “Manual of Histological Staining Method of the Armed Forces Institute of Pathology,” 3rd edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulteka V. Mikel, Editor, Armed Forces Institute of Pathology, American Registry of Pathology, Washington, D.C.). One of skill in the art will appreciate that the choice of a fixative is determined by the purpose for which the tissue is to be histologically stained or otherwise analyzed. One of skill in the art will also appreciate that the length of fixation depends upon the size of the tissue sample and the fixative used. By way of example, neutral buffeted formalin, Bouin's or paraformaldehyde, may be used to fix a tissue sample.

Generally, the tissue sample is first fixed and is then dehydrated through arm ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology”, supra). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology”, supra). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology”, supra). Alternatively, commercially available deparaffinizing non-organic agents such as Hemo-De7 (CMS, Houston, Tex.) may be used.

Subsequent to tissue preparation, a tissue section may be subjected to immunohistochemistry (IHC). IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, New York, Pubs. (1991) for example and radioactivity can be measured using scintillation counting.

(b) Colloidal gold particles.

(c) Fluorescent labels including, but are not limited to, rate earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter.

(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired, For example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g. using a microscope.

While not being bound by the following parameters. protein staining intensity criteria may be evaluated as illustrated by the following chart:

Protein Staining Intensity Criteria

| Staining Pattern | Score |
|---|---|
| No staining is observed in tumor cells. | 0 |
| A faint/barely perceptible staining is detected | 1+ |
| in tumor cells. | |
| A weak to moderate complete staining is observed | 2+ |
| in tumor cells. | |
| A moderate to strong complete staining is | 3+ |
| observed in tumor cells. | |
| A strong to very strong complete staining is | 4+ |
| observed in tumor cells. | |

Other methods for identifying a cell that expresses ROR1 are also available to the skilled artisan. In one embodiment, an assay for identifying a cell that expresses a ROR1 gene comprises detecting the presence of ROR1 mRNA in the cell. Methods for the detection of particular mRNAs in cells are well known and include, for example, hybridization assays using complementary DNA probes (such as in situ hybridization using labeled ROR1 riboprobes, Northern blot and related techniques) and various nucleic acid amplification assays (such as RT-PCR using complementary primers specific for ROR1, and other amplification type detection methods, such as, for example, branched DNA, SISBA, TMA and the like). Alternatively, an assay for identifying a cell that expresses a ROR1 gene comprises detecting the presence of ROR1 protein in the cell or secreted by the cell. Various methods for the detection of proteins are well known in the art End may be employed for the detection of ROR1 proteins and ROR1 expressing cells.

ROR1 expression analysis may also be useful as a tool for identifying and evaluating agents that modulate ROR1 gene expression. For example, ROR1 expression is significantly upregulated in breast cancer, is also aberrantly expressed in other cancers. Identification of a molecule or biological agent that could inhibit ROR1 expression or over-expression in cancer cells may be of therapeutic value. Such an agent may be identified by using a screen that quantifies ROR1 expression by RT-PCR, nucleic acid hybridization or antibody binding.

### Monitoring the Status of ROR1 and its Products

Assays that evaluate the status of the ROR1 gene and ROR1 gene products in an individual may provide information on the growth or oncogenic potential of a biological sample from this individual. For example, because ROR1 mRNA is so highly expressed in certain breast cancer cells as compared to normal breast tissue, assays that evaluate the relative levels of ROR1 mRNA transcripts or proteins in a biological sample can be used to diagnose a disease associated with ROR1 disregulation such as cancer and may provide prognostic information that can for example be useful in defining appropriate therapeutic options. Similarly, assays that evaluate the integrity ROR1 nucleotide and amino acid sequences in a biological sample, can also be used in this context.

The finding that ROR1 mRNA is so highly expressed in certain breast cancer subtypes provides evidence that this gene is associated with disregulated cell growth and therefore identifies this gene and its products as targets that the skilled artisan can use to evaluate biological samples from individuals suspected of having a disease associated with ROR1 disregulation. In this context, the evaluation of the status of ROR1 gene and its products can be used to gain information on the disease potential of a tissue sample.

The term “status” in this context is used according to its art accepted meaning and refers to the condition a gene and its products including, but not limited to the integrity and/or methylation of a gene including its regulatory sequences, the location of expressed gene products (including the location of ROR1 expressing cells), the presence, level, and biological activity of expressed gene products (such as ROR1 mRNA polynucleotides and polypeptides), the presence or absence of transcriptional and translational modifications to expressed gene products as well as associations of expressed gene products with other biological molecules such as protein binding partners. Alterations in the status of ROR1 can be evaluated by a wide variety of methodologies well known in the art, typically those discussed below. Typically an alteration in the status of ROR1 comprises a change in the location of ROR1 expressing cells, an increase in ROR1 mRNA and/or protein expression and/or the association or dissociation of ROR1 with a binding partner.

The expression profile of ROR1 makes it a potential diagnostic market for local and/or metastasized breast cancer disease. In particular, the status of ROR1 may provide information useful for predicting susceptibility to particular disease stage or subtype, progression, and/or tumor aggressiveness. The invention provides methods and assays for determining ROR1 status and diagnosing cancers that express ROR1, such as cancers of the breast. ROR1 status in patient samples may be analyzed by a number of means well known in the art, including without limitation, immunohistochemical analysis, in situ hybridization, RT-PCR analysis on laser capture micro-dissected samples, western blot analysis of clinical samples and cell lines, and tissue array analysis. Typical protocols for evaluating the status of the ROR1 gene and gene products can be found, for example in Ausubul et al. eds., 1995, Current Protocols In Molecular Biology, Units 2 [Northern Blotting], 4 [Southern Blotting], 15 [Immunoblotting] and 18 [PCR Analysis].

As described above, the status of ROR1 in a biological sample can be examined by a number of well known procedures in the art. For example, the status of ROR1 in a biological sample taken from a specific location in the body can be examined by evaluating the sample for the presence or absence of ROR1 expressing cells (e.g. those that express ROR1 mRNAs or proteins). This examination can provide evidence of disregulated cellular growth for example, when ROR1 expressing breast cells are found in a biological sample that does not normally contain such cells (such as a lymph node, bone or spleen). Such alterations in the status of ROR1 in a biological sample are often associated with disregulated cellular growth. Specifically, one indicator of disregulated cellular growth is the metastases of cancer cells from an organ of origin (such as the breast gland) to a different area of the body (such as a lymph node). In this context, evidence of disregulated cellular growth is important for example because occult lymph node metastases can be detected in a substantial proportion of patients with breast cancer, and such metastases are associated with known predictors of disease progression (see, e.g. Gipponni et al., J Surg Oncol. 2004 Mat 1; 85(3):102-111).

In one aspect, the invention provides methods for monitoring ROR1 gene products by determining the status of ROR1 gene products expressed by cells in a test tissue sample from an individual suspected of having a disease associated with disregulated cell growth (such as hyperplasia or cancer) and then comparing the status so determined to the status of ROR1 gene products in a corresponding normal sample, the presence of aberrant ROR1 gene products in the test sample relative to the normal sample providing an indication of the presence of disregulated cell growth within the cells of the individual.

In another aspect, the invention provides assays useful in determining the presence of cancer in an individual, comprising detecting a significant increase in ROR1 mRNA or protein expression in a test cell or tissue sample relative to expression levels in the corresponding normal cell or tissue. The presence of ROR1 mRNA may, for example, be evaluated in tissue samples including but not limited to breast cancer subtypes such as basal and BRCA 1 breast cancer subtypes (see, e.g. Sortlie et al., PNAS (2001), 98(19): 10869-10874), etc. The presence of significant ROR1 expression in any of these tissues may be useful to indicate the emergence, presence and/or severity of these cancers, since the corresponding normal tissues do not express ROR1 mRNA or express it at lower levels.

In a related embodiment, ROR1 status may be determined at the protein level rather than at the nucleic acid level. For example, such a method or assay would comprise determining the level of ROR1 protein expressed by cells in a test tissue sample and comparing the level so determined to the level of ROR1 expressed in a corresponding normal sample. In one embodiment, the presence of ROR1 protein is evaluated, for example, using immunohistochemical methods. ROR1 antibodies or binding partners capable of detecting ROR1 protein expression may be used in a variety of assay formats well known in the art for this purpose.

In other related embodiments, one can evaluate the integrity ROR1 nucleotide and amino acid sequences in a biological sample in order to identify perturbations in the structure of these molecules such as insertions, deletions, substitutions and the like. Such embodiments are useful because perturbations in the nucleotide and amino acid sequences are observed in a large number of proteins associated with a growth disregulated phenotype (see, e.g., Mattogi et al., 1999, J. Cutan. Pathol. 26(8):369-378). In this context, a wide variety of assays for observing perturbations in nucleotide and amino acid sequences are well known in the art. For example, the size and structure of nucleic acid or amino acid sequences of ROR1 gene products may be observed by the Northern, Southern, Western, PCR and DNA sequencing protocols discussed herein. In addition, other methods for observing perturbations in nucleotide and amino acid sequences such as single strand conformation polymorphism analysis are well known in the art (see, e.g., U.S. Pat. Nos. 5,382,510 and 5,952,170).

In another embodiment, one can examine the methylation status of the ROR1 gene in a biological sample. Aberrant demethylation and/or hypermethylation of CpG islands in gene 5′ regulatory regions frequently occurs in immortalized and transformed cells and can result in altered expression of various genes. For example, promoter hypermethylation of the pi-class glutathione S-transferase (a protein expressed in normal prostate but not expressed in >90% of prostate carcinomas) appears to permanently silence transcription of this gene and is the most frequently detected genomic alteration in prostate carcinomas (De Marzo et al., Am. J. Pathol. 155(6): 1985-1992 (1999)). In addition, this alteration is present in at least 70% of cases of high-grade prostatic intraepithelial neoplasia (PIN) (Brooks et al, Cancer Epidemiol. Biomarkers Prev., 1998, 7:531-536). In another example, expression of the LAGE-I tumor specific gene (which is not expressed in normal prostate but is expressed in 25-50% of prostate cancers) is induced by deoxy-azacytidine in lymphoblastoid cells, suggesting that tumoral expression is due to demethylation (Lethe et al., Int. J. Cancer 76(6): 903-908 (1998)). In this context, a variety of assays for examining methylation status of a gene are well known in the art. For example, one can utilize in Southern hybridization approaches methylation-sensitive restriction enzymes which can not cleave sequences that contain methylated CpG sites in order to assess the overall methylation status of CpG islands. In addition, MSP (methylation specific PCR) can rapidly profile the methylation status of all the CpG sites present in a CpG island of a given gene. This procedure involves initial modification of DNA by sodium bisulfite (which will convert all unmethylated cytosines to uracil) followed by amplification using primers specific for methylated versus unmethylated DNA. Protocols involving methylation interference can also be found for example in Current Protocols In Molecular Biology, Units 12, Frederick M. Ausubul et al. eds., 1995.

Gene amplification provides an additional method of assessing the status of ROR1, a locus that maps to lp31, a region shown to be perturbed in a variety of cancers. Gene amplification may be measured in a sample directly, for example, by conventional Southern blotting, Northern blotting to quantitate the transcription of mRNA (Thomas, 1980, Proc. Natl. Acad. Sci. USA, 77:5201-5205), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

In addition to the tissues discussed above, peripheral blood may be conveniently assayed for the presence of cancer cells, including but not limited to breast cancers, using for example, Northern or RT-PCR analysis to detect ROR1 expression. The presence of RT-PCR amplifiable ROR1 mRNA provides an indication of the presence of the cancer. RT-PCR detection assays for tumor cells in peripheral blood are currently being evaluated for use in the diagnosis and management of a number of human solid tumors.

A related aspect of the invention is directed to predicting susceptibility to developing cancer in an individual. In one embodiment, a method for predicting susceptibility to cancer comprises detecting ROR1 mRNA or ROR1 protein in a tissue sample, its presence indicating susceptibility to cancer, wherein the degree of ROR1 mRNA expression present is proportional to the degree of susceptibility. In a specific embodiment, the presence of ROR1 in breast tissue is examined, with the presence of ROR1 in the sample providing an indication of breast cancer susceptibility (or the emergence or existence of a breast tumor and/or the emergence or existence of a specific breast tumor subtype). In another specific embodiment, the presence of ROR1 in tissue is examined, with the presence of ROR1 in the sample providing an indication of cancer susceptibility (or the emergence or existence of a tumor). In a closely related embodiment, one can evaluate the integrity ROR1 nucleotide and amino acid sequences in a biological sample in order to identify perturbations in the structure of these molecules such as insertions, deletions, substitutions and the like, with the presence of one or more perturbations in ROR1 gene products in the sample providing an indication of cancer susceptibility (or the emergence or existence of a tumor).

Yet another related aspect of the invention is directed to methods for gauging tumor aggressiveness. In one embodiment, a method for gauging aggressiveness of a tumor comprises determining the level of ROR1 mRNA or ROR1 protein expressed by cells in a sample of the tumor, comparing the level so determined to the level of ROR1 mRNA or ROR1 protein expressed in a corresponding normal tissue taken from the same individual or a normal tissue reference sample, wherein the degree of ROR1 mRNA or ROR1 protein expression in the tumor sample relative to the normal sample indicates the degree of aggressiveness. In a specific embodiment, aggressiveness of a tumor is evaluated by determining the extent to which ROR1 is expressed in the tumor cells, with higher expression levels indicating mote aggressive tumors. In a closely related embodiment, one can evaluate the integrity of ROR1 nucleotide and amino acid sequences in a biological sample in order to identify perturbations in the structure of these molecules such as insertions, deletions, substitutions and the like, with the presence of one or more perturbations indicating more aggressive tumors.

Yet another related aspect of the invention is directed to methods for observing the progression of a malignancy in an individual over time. In one embodiment, methods for observing the progression of a malignancy in an individual over time comprise determining the level of ROR1 mRNA or ROR1 protein expressed by cells in a sample of the tumor, comparing the level so determined to the level of ROR1 mRNA or ROR1 protein expressed in an equivalent tissue sample taken from the same individual at a different time, wherein the degree of ROR1 mRNA or ROR1 protein expression in the tumor sample over time provides information on the progression of the cancer. In a specific embodiment, the progression of a cancer is evaluated by determining the extent to which ROR1 expression in the tumor cells alters over time, with higher expression levels indicating a progression of the cancer. In a closely related embodiment, one can evaluate the integrity ROR1 nucleotide and amino acid sequences in a biological sample in order to identify perturbations in the structure of these molecules such as insertions, deletions, substitutions and the like, with the presence of one or more perturbations indicating a progression of the cancer.

The above diagnostic approaches may be combined with any one of a wide variety of prognostic and diagnostic protocols known in the art. For example, another embodiment of the invention disclosed herein is directed to methods for observing a coincidence between the expression of ROR1 gene and ROR1 gene products (or perturbations in ROR1 gene and ROR1 gene products) and a factor that is associated with malignancy as a means of diagnosing and prognosticating the status of a tissue sample. In this context, a wide variety of factors associated with malignancy may be utilized such as the expression of genes otherwise associated with malignancy (including Her-2 and BRCA 1 and 2 expression) as well as gross cytological observations (see e.g. Bocking et al., 1984, Anal. Quant. Cytol. 6(2):74-88; Eptsein, 1995, Hum. Pathol. 26(2):223-9; Thorson et al., 1998, Mod. Pathol. 11(6):543-51; Baisden et al., 1999, Am. J. Surg. Pathol. 23(8):918-24). Methods for observing a coincidence between the expression of ROR1 gene and ROR1 gene products (or perturbations in ROR1 gene and ROR1 gene products) and an additional factor that is associated with malignancy are useful, for example, because the presence of a set or constellation of specific factors that coincide provides information crucial for diagnosing and prognosticating the status of a tissue sample.

In a typical embodiment, methods for observing a coincidence between the expression of ROR1 gene and ROR1 gene products (or perturbations in ROR1 gene and ROR1 gene products) and a factor that is associated with malignancy entails detecting the overexpression of ROR1 mRNA or protein in a tissue sample, detecting the overexpression of BRCA 1 or 2 mRNA or protein in a tissue sample, and observing a coincidence of ROR1 mRNA or protein and BRCA mRNA or protein overexpression. In another specific embodiment, the expression of ROR1 and Her-2 mRNA in breast tissue is examined. In a common embodiment, the coincidence of ROR1 and Her-2 or BRCA 1 or 2 mRNA overexpression in the sample provides an indication of breast cancer, breast cancer subtype, breast cancer susceptibility or the emergence or existence of a breast tumor.

Methods for detecting and quantifying the expression of ROR1 mRNA or protein are described herein and use of standard nucleic acid and protein detection and quantification technologies is well known in the art. Standard methods for the detection and quantification of ROR1 mRNA include in situ hybridization using labeled ROR1 riboprobes, Northern blot and related techniques using ROR1 polynucleotide probes, RT-PCR analysis using primers specific for ROR1, and other amplification type detection methods, such as, for example, branched DNA, SISBA, TMA and the like. In a specific embodiment, RT-PCR may be used to detect and quantify ROR1 mRNA expression as described in the Examples. Any number of primers capable of amplifying ROR1 may be used for this purpose. Standard methods for the detection and quantification of protein may be used for this purpose. In a specific embodiment, polyclonal or monoclonal antibodies specifically reactive with the wild-type ROR1 protein may be used in an immunohistochemical assay of biopsied tissue.

The invention has a number of embodiments. One embodiment is a method of examining a test biological sample comprising a human breast cell for evidence of altered cell growth that is indicative of a breast cancer by evaluating the levels of orphan receptor tyrosine kinase (ROR1) polynucleotides that encode the ROR1 polypeptide shown in SEQ ID NO: 2 in the biological sample, wherein an increase in the levels of the ROR1 polynucleotides in the test sample relative to a normal breast tissue sample provide evidence of altered cell growth that is indicative of a breast cancer; and wherein the levels of the ROR1 polynucleotides in the cell are evaluated by contacting the sample with a ROR1 complementary polynucleotide that hybridizes to a ROR1 nucleotide sequence shown in SEQ ID NO: 1, or a complement thereof, and evaluating the presence of a hybridization complex formed by the hybridization of the ROR1 complementary polynucleotide with the ROR1 polynucleotides in the test biological sample.

A related embodiment is a method of examining a human breast cell for evidence of altered cell growth that is associated with or provides evidence of a breast cancer by evaluating the levels of orphan receptor tyrosine kinase (ROR1) polynucleotides that encode the ROR1 polypeptide shown in SEQ ID NO: 2 in the human breast cell, wherein an increase in the levels of the ROR1 polynucleotides (e.g. mRNAs and genomic sequences) in the human breast cell relative to a normal human breast cell provides evidence of altered cell growth that is associated with or provides evidence of a breast cancer; and wherein the levels of the ROR1 polynucleotides in the human breast cell are evaluated by contacting the endogenous ROR1 polynucleotide sequences in the human breast cell with a ROR1 complementary polynucleotide the ROR1 complementary polynucleotide (e.g. a probe labelled with a detectable marker or a PCR primer) and which specifically hybridizes to a ROR1 nucleotide sequence shown in SEQ ID NO: 1 and evaluating the presence of a hybridization complex formed by the hybridization of the ROR1 complementary polynucleotide with the ROR1 polynucleotides in the sample (e.g. via Northern analysis or PCR) so that evidence of altered cell growth that is associated with or provides evidence of a breast cancer is examined. Certain embodiments of the invention include the step of examining the expression of Her-2 (SEQ ID NO: 3), EGFR (SEQ ID NO: 4), VEGF (SEQ ID NO: 5), FMS-like tyrosine kinase (SEQ ID NO: 6), MYC (SEQ ID NO: 7), urokinase plasminogen activator (SEQ ID NO: 8), plasminogen activator inhibitor (SEQ ID NO: 9), BRCA1 (SEQ ID NO: 10) or BRCA2 (SEQ ID NO: 11) polynucleotides in the test biological sample.

In some embodiments of the invention, the increase in the levels of the ROR1 polynucleotides in the human breast cell relative to a normal human breast cell that provides evidence of altered cell growth is quantified, for example, as being at least a 100% (1 fold) increase, or a 200% (2 fold), 4 fold, 8 fold, 15 fold, 30 fold, 60 fold, or a 120 fold increase in the relative levels of the ROR1 polynucleotides. In the quantitative mRNA analyses disclosed herein (see, e.g. FIG. 5), the increase in the levels of the ROR1 mRNAs in the cells tested ranged from a 15 fold increase (e.g. in the BT-20 cell line) to a 120 fold increase in the HCC1187 cell line. The average increase in the levels of the ROR1 polynucleotides in the overexpressing cell lines as compared to the observed expression in luminal breast cancer cell lines is 43 fold. The normalized standard that can be used as a comparative reference of ROR1 expression can for example be obtained from normal breast tissue taken from the same individual, or a normal tissue reference sample taken from a healthy individual. Alternatively, a normalized standard can be a numerical range of normal ROR1 expression that is obtained from a statistical sampling of normal cells from a population of individuals. In certain embodiments of the invention, the normalized standard is derived by comparing ROR1 expression to a control gene that is expressed in the same cellular environment at relatively stable levels (e.g. a housekeeping gene such as an actin).

Immortalized, non-malignant breast cell lines appear to be of basal origin and also express ROR1 polynucleotides at levels significantly higher than luminal breast cancer cells. In this context, the level of ROR1 polynucleotide expression is observed to be higher in basal breast cancer cells as compared to non-malignant basal cells, with an average increase in ROR1 polynucleotide expression being a 7 fold increase. While there are no continuously growing non-malignant luminal cells available, the analyses of luminal breast cancer and normal tissues described herein suggests that the expression of ROR1 polynucleotides in normal luminal mammary cells is very low or undetectable. When ROR1 expression in primary breast cancer is compares to breast cell lines are calculated as a log ratio, the average log ratio of the 12 highest ROR1 expressing cell lines is 0.40 (with a range from 0.12 to 0.9). The average log ratio of the 5 basal ROR1 positive primary breast cancers is 0.26 (with a range from 0.21 to 0.32). The consistency of these calculations is supported by the observation that when compared against the same reference (pure tumor cell lines) the breast tumors have similar but slightly lower ROR1 expression levels than those observed in pure cell lines. Without being bound by a specific theory, these observations are consistent with a simple dilution effect because the tumor cells in the primary tumor occur in a complex mixture of cell types (including those that are known not to express ROR1).

In certain embodiments of the invention, the breast cancer is of the basal subtype. As is known in the art, cancers of the breast can be group into a number of distinct subtypes, including a basal subtype (see, e.g. see, e.g. Sorlie et al., PNAS (2001), 98(19): 10869-10874). In particular, mammary ducts are bilayered structures composed of a luminal layer and a myoepithelial layer that adhere to a basement membrane. The term basal subtype is an art accepted term that refers to certain cancers that arise from the basal layer of the stratified epithelia (see, e.g. FIG. 1 in Wilson et al. Breast Cancer Research Vol 6 No. 5: 192-200 (2004)). Breast carcinomas of the basal subtype reside in the basal layer of the ductal epithelium of the breast as opposed to the apical or luminal layers. Such cancers have distinct cytological features and gene expression profiles such as an intermediate filament profile (cytokeratins) first observed in the basal cells of the skin. In particular, basal cells in the skin are known to express certain cytokeratins (i.e. K5/6, K7, K17, K14) which are found in complex epithelia as opposed to K8, K18, K19 which are found in simple, or glandular epithelia.

A subtype of breast cancer (e.g. one with basal cell properties) can be readily determined via pathology-IHC data and/or the Stanford breast tumor profiling data disclosed herein. For example, Wetzels et al, Am J Path. (1991) 138: p751-63 which is incorporated herein by reference describe basal cell-specific and hyperproliferations-related keratins in human breast cancer. This study found that 15% (n=115) of invasive breast cancers were positive for basal cytokeratins 14 and 17. In addition, Bartek et al., Int J. Cancer (1985) 36:299-306 which is incorporated herein by reference also teach the characterization of breast cancer subtypes using patterns of expression of K19 in human breast tissues and tumors. Conversely, most medullary and poorly differentiated ductal carcinomas were negative for cytokeratin 19 while moderately and well-differentiated ductal, invasive lobular, tubular and most mucinous carcinomas were positive with both K19 Abs. In addition, P-Cadherin (CDH3) (SEQ ID NO: 12) and Desmosomal Cadherins are expressed in Basal Layer of Breast Ducts and P-Cadherin mRNA is overexpressed in the basal and BRCA1 subtypes. This provides confirmatory evidence that the Group 4 and BRCA1 tumor groups share many molecular properties associated with cell type origin.

Paredes et al., Pathol. Res. Pract. 2002: 198(12): 795-801 which is incorporated herein by reference also investigate the expression of P cadherin in breast carcinoma subtypes and correlate it with estrogen receptor (ER) status. 73 ductal carcinomas in situ (DCIS) and 149 invasive carcinomas of the breast were selected and examined for the expression of P-cadherin as well as other biologic markers. P-cadherin expression showed a strong inverse correlation with estrogen receptor (ER) expression in both types of breast carcinoma (in situ and invasive). P-cadherin-positive and ER-negative tumors were related to a higher histologic grade, a high proliferation rate, and expression of c-erbB-2. This demonstrates that P-cadherin identifies a subgroup of breast carcinomas that lacks ER expression, and correlates with higher proliferation rates and other predictors of aggressive behavior. See also, Gamallo et al., Mod. Pathol. 2001: 14(7): 650-4; Kovacs et al., J Clin Pathol 2003 February; 56(2):139-41; and Peralta et al., Cancer 1999 Oct. 1; 86(7):1263-72 which are incorporated herein by reference.

In certain embodiments of the invention, the breast cancer is of the BRCA1 subtype. In particular, as is known in the art, cancers of the breast can be group into a number of distinct subtypes, including a BRCA1 subtype (see, e.g. see, e.g. Sorlie et al., PNAS (2001), 98(19): 10869-10874). In this context, a breast cancer of the BRCA1 subtype is characterized as having a mutation in the BRCA1 gene. A variety of distinct BRCA1 mutations are known to occur in multiple tissues and include substitutions, deletions and missense mutations (see, e.g. Wagner et al., Int J. Cancer. 1998 Jul. 29; 77(3):354-60; Chang et al., Breast Cancer Res Treat. 2001 September; 69(2):101-13; and Foulkes et al., Cancer Res. 2004 Feb. 1; 64(3):830-5; and Aghmesheh et al., Gynecol Oncol. 2005 April; 97(1):16-25 which are incorporated herein by reference). The Basal and BRCA1 cancers are related by cellular origin and molecular pathogenesis and the over-expression of ROR1 is an important alteration involved in the pathogenesis of these two tumor groups.

FIG. 5F and FIG. 5G show the detection of endogenous ROR1 protein on the surface of CAL51 cells using anti-ROR1 rabbit polyclonal sera, with SKBR cells serving as a comparative cell line. When compared to the ROR1 mRNA expression data shown for example in FIG. 5B, these studies with anti-ROR1 rabbit polyclonal sera demonstrate that ROR1 mRNA expression levels correlate with ROR1 protein expression levels. The mRNA/protein expression correlative data presented in these figures is consistent with other observations of ROR1 mRNA and protein expression. For example, Paganoni et al., in J. Neuroscience Research 73: 429-440 (2003) (which is incorporated herein by reference) teach that observations of ROR1 mRNA expression examined via in situ hybridization and/or PCR analyses correlate with observations of ROR1 protein expression examined via immunohistochemical and/or Western analyses in a variety of cells that express ROR1. In addition, Paganoni et al., in GLIA 46: 456-466 (2004) (which is incorporated herein by reference) teach that both the ROR1 and ROR2 mRNAs and the ROR1 and ROR2 proteins are expressed in vivo in early stages in brain development. In this GLIA article Paganoni et al. further teach that not only ROR1 and ROR2 mRNAs, but also ROR proteins, are highly expressed in certain cultured cells. The observation that ROR1 mRNA expression levels correlate with ROR1 protein expression levels is further supported by data presented herein that breast cancer cells that overexpress ROR1 exhibit for example, a specific basal phenotype and have a poor prognosis as compared to cells that do not overexpress ROR1 (characteristics known in the art to be influenced by the function of translated proteins).

Another embodiment of the invention is a method of examining a test biological sample comprising a human breast cell for evidence of altered cell growth that is indicative of a breast cancer, the method comprising evaluating the levels of orphan receptor tyrosine kinase (ROR1) polypeptides having the sequence shown in SEQ ID NO: 2 in the biological sample, wherein an increase in the levels of the ROR1 polypeptides in the test sample relative to a normal breast tissue sample provide evidence of altered cell growth that is indicative of a breast cancer; and wherein the levels of the ROR1 polypeptides in the cell are evaluated by contacting the sample with an antibody that immunospecifically binds to a ROR1 polypeptide sequence shown in SEQ ID NO: 2 and evaluating the presence of a complex formed by the binding of the antibody with the ROR1 polypeptides in the sample.

A related embodiment of the invention is a method of examining a human breast cell (e.g. from a biopsy) that is suspected of being cancerous for evidence of altered cell growth that is indicative of a breast cancer, the method comprising evaluating the levels of orphan receptor tyrosine kinase (ROR1) polypeptides having the sequence shown in SEQ ID NO: 2 in the breast cell, wherein an increase in the levels of the ROR1 polypeptides in the human breast cell relative to a normal breast cell (e.g. a normal cell from the individual providing the human breast cell) provide evidence of altered cell growth that is indicative of a breast cancer; and wherein the levels of the ROR1 polypeptides in the cell are evaluated by contacting the sample with an antibody (e.g. one labelled with a detectable market) that immunospecifically binds to a ROR1 polypeptide sequence shown in SEQ ID NO: 2 and evaluating the presence of a complex formed by the binding of the antibody with the ROR1 polypeptides in the sample. Typically the presence of a complex is evaluated by a method selected from the group consisting of ELISA analysis, Western analysis and immunohistochemistry. Optionally, the breast cancer is of the basal or the BRCA 1 subtype.

Yet another embodiment of the invention is a method of examining a test human cell for evidence of a chromosomal abnormality that is indicative of a human cancer by comparing orphan receptor tyrosine kinase (ROR1) polynucleotide sequences from band p31 of chromosome 1 in a normal cell to ROR1 polynucleotide sequences from band p31 of chromosome 1, band p31 on chromosome 1 in the test human cell to identify an amplification or an alteration (e.g. a deletion, insertion, substitution or missense mutation) of the ROR1 polynucleotide sequences in the test human cell, wherein an amplification or an alteration of the ROR1 polynucleotide sequences in the test human cell provides evidence of a chromosomal abnormality that is indicative of a human cancer. In such methods chromosome 1, band p31 in the test human cell is typically evaluated by contacting the ROR1 polynucleotide sequences in the test human cell sample with a ROR1 complementary polynucleotide that specifically hybridizes to a ROR1 nucleotide sequence shown in SEQ ID NO: 1, or a complement thereof, and evaluating the presence of a hybridization complex formed by the hybridization of the ROR1 complementary polynucleotide with the ROR1 polynucleotide sequences in the test human cell (e.g. by Northern analysis, Southern analysis or polymerase chain reaction analysis).

Identifying Molecules that Interact with ROR1

The ROR1 protein sequences disclosed herein allow the skilled artisan to identify molecules that interact with them via any one of a variety of art accepted protocols. For example one can utilize one of the variety of so-called interaction trap systems (also referred to as the “two-hybrid assay”). In such systems, molecules that interact reconstitute a transcription factor and direct expression of a reporter gene, the expression of which is then assayed. Typical systems identify protein-protein interactions in vivo through reconstitution of a eukaryotic transcriptional activator and are disclosed for example in U.S. Pat. Nos. 5,955,280, 5,925,523, 5,846,722 and 6,004,746.

Alternatively one can identify molecules that interact with ROR1 protein sequences by screening peptide libraries. In such methods, peptides that bind to selected receptor molecules such as ROR1 are identified by screening libraries that encode a random or controlled collection of amino acids. Peptides encoded by the libraries are expressed as fusion proteins of bacteriophage coat proteins, and bacteriophage particles are then screened against the receptors of interest. Peptides having a wide variety of uses, such as therapeutic or diagnostic reagents, may thus be identified without any prior information on the structure of the expected ligand or receptor molecule. Typical peptide libraries and screening methods that can be used to identify molecules that interact with ROR1 protein sequences are disclosed for example in U.S. Pat. Nos. 5,723,286 and 5,733,731.

Alternatively, cell lines expressing ROR1 can be used to identify protein-protein interactions mediated by ROR1. This possibility can be examined using immunoprecipitation techniques as shown by others (Hamilton, B J., et al., 1999, Biochem. Biophys. Res. Commun. 261:646-51). Typically ROR1 protein can be immunoprecipitated from ROR1 expressing breast cancer cell lines using anti-ROR1 antibodies. Alternatively, antibodies against His-tag can be used in cell line engineered to express ROR1 (vectors mentioned above). The immunoprecipitated complex can be examined for protein association by procedures such as western blotting, ³⁵S-methionine labeling of proteins, protein microsequencing, silver staining and two dimensional gel electrophoresis.

Related embodiments of such screening assays include methods for identifying small molecules that interact with ROR1. Typical methods are discussed for example in U.S. Pat. No. 5,928,868 and include methods for forming hybrid ligands in which at least one ligand is a small molecule. In an illustrative embodiments, the hybrid ligand is introduced into cells that in turn contain a first and a second expression vector. Each expression vector includes DNA for expressing a hybrid protein that encodes a target protein linked to a coding sequence for a transcriptional module. The cells further contains a reporter gene, the expression of which is conditioned on the proximity of the first and second hybrid proteins to each other, an event that occurs only if the hybrid ligand binds to target sites on both hybrid proteins. Those cells that express the reporter gene are selected and the unknown small molecule or the unknown hybrid protein is identified.

A typical embodiment of this invention consists of a method of screening for a molecule that interacts with a ROR1 amino acid sequence shown in FIG. 1, comprising the steps of contacting a population of molecules with the ROR1 amino acid sequence, allowing the population of molecules and the ROR1 amino acid sequence to interact under conditions that facilitate an interaction, determining the presence of a molecule that interacts with the ROR1 amino acid sequence and then separating molecules that do not interact with the ROR1 amino acid sequence from molecules that do interact with the ROR1 amino acid sequence. In a specific embodiment, the method further includes purifying a molecule that interacts with the ROR1 amino acid sequence. In one embodiment, the ROR1 amino acid sequence is contacted with a library of peptides.

### Therapeutic Methods and Compositions

The identification of ROR1 as a gene that is highly expressed in subtypes of cancers of the breast (and possibly other cancers), opens a number of therapeutic approaches to the treatment of such cancers. As discussed above, it is possible that ROR1 is secreted from cancer cells and in this way modulates proliferation signals. Its potential role as a transcription factor and its high expression in breast cancer makes it a potential target for small molecule-mediated therapy.

Accordingly, therapeutic approaches aimed at inhibiting the activity of the ROR1 protein are expected to be useful for patients suffering from breast cancer and other cancers expressing ROR1.

### ROR1 as a Target for Antibody-Based Therapy

As disclosed herein, ROR1 is a cell surface protein that is overexpressed in certain pathologies such as cancers of the breast. The structural features of ROR1 indicate that this molecule is an attractive target for antibody-based therapeutic strategies. Because ROR1 is expressed by cancer cells of various Lineages and not by corresponding normal cells, systemic administration of ROR1-immunoreactive compositions would be expected to exhibit excellent sensitivity without toxic, non-specific and/or non-target effects caused by binding of die immunotherapeutic molecule to non-target organs and tissues. Antibodies specifically reactive with domains of ROR1 can be useful to treat ROR1-expressing cancers systemically, either as conjugates with a toxin or therapeutic agent, or as naked antibodies capable of inhibiting cell proliferation or function.

As is known in the art, antibodies to cell surface proteins can be used in therapeutic methods which preferentially kill cells that these express cell surface proteins, particularly in situations where cell surface protein is overexpressed in the pathological cells versus the normal cells in a patients body (e.g. HER2). Well known methodologies using such antibodies take advantage of the ability of such antibodies to activate the complement cascade and/ort mediate antibody dependent cellular cytotoxicity in a patient treated with an effective amount of the antibody. Alternative methodologies include the use of an immunotoxin which is a conjugate of a cytotoxic moiety and one of these antibodies. The amount of experimentation need to assess the ability of an anti-ROR1 antibody to inhibit the growth of any cell examined is minor and follows well established protocols in the art. Moreover, the ability of an antibody to kill a cell expressing on its surface a protein recognized by that antibody and having the specific characteristics of ROR1 (e.g. having an expression pattern and structure etc. similar to proteins such as HER2) follows well established scientific principles. Consequently the ability of an ROR1 antibody to inhibit the growth of and/or kill any cell type can be determined with minimal experimentation.

ROR1 antibodies can be introduced into a patient such that the antibody binds to ROR1 and modulates or perturbs a function such as an interaction with receptors and ligands of the frizzled family and consequently mediates the destruction of the cells and the tumor and/or inhibits the growth of the cells or the tumor. Mechanisms by which such antibodies exert a therapeutic effect may include complement-mediated cytolysis, antibody-dependent cellular cytotoxicity, modulating the physiological function of ROR1, inhibiting ligand binding or signal transduction pathways, modulating tumor cell differentiation, altering tumor angiogenesis factor profiles, and/or by inducing apoptosis. ROR1 antibodies can be conjugated to toxic or therapeutic agents and used to deliver the toxic or therapeutic agent directly to ROR1-bearing tumor cells. Examples of toxic agents include, but are not limited to, calchemicin, maytansin oids, radioisotopes such as ¹³¹I, ytrium, and bismuth.

Cancer immunotherapy using anti-ROR1 antibodies may follow the teachings generated from various approaches that have been successfully employed in the treatment of other types of cancer, including but not limited to colon cancer (Arlen et al., 1998, Crit. Rev. Immunol. 18:133-138), multiple myeloma (Ozaki et al., 1997, Blood 90:3179-3186; Tsunenari et al., 1997, Blood 90:2437-2444), gastric cancer (Kasprzyk et al., 1992, Cancer Res. 52:2771-2776), B-cell lymphoma (Funakoshi et al., 1996, J. Immunother. Emphasis Tumor Immunol. 19:93-101), leukemia (Zhong et al., 1996, Leuk. Res. 20:581-589), colorectal cancer (Moun et al., 1994, Cancer Res. 54:6160-6166; Velders et al., 1995, Cancer Res. 55:4398-4403), and breast cancer (Shepard et al., 1991, J. Clin. Immunol. 11:117-127). Some therapeutic approaches involve conjugation of naked antibody to a toxin, such as the conjugation of ¹³¹I to anti-CD20 antibodies (e.g., Rituxan™, IDEC Pharmaceuticals Corp.), while others involve co-administration of antibodies and other therapeutic agents, such as Herceptin™ (trastuzumab) with paclitaxel (Genentech, Inc.). For treatment of breast cancer, for example, ROR1 antibodies can be administered in conjunction with radiation, chemotherapy or hormone ablation.

Although ROR1 antibody therapy may be useful for all stages of cancer, antibody therapy may be particularly appropriate in advanced or metastatic cancers. Treatment with the antibody therapy of the invention may be indicated for patients who have received previously one or more chemotherapy, while combing the antibody therapy of the invention with a chemotherapeutic or radiation regimen may be preferred for patients who have not received chemotherapeutic treatment. Additionally, antibody therapy may enable the use of reduced dosages of concomitant chemotherapy, particularly for patients who do not tolerate the toxicity of the chemotherapeutic agent very well.

It may be desirable for some cancer patients to be evaluated for the presence and level of ROR1 expression, preferably using immunohistochemical assessments of tumor tissue, quantitative ROR1 imaging, or other techniques capable of reliably indicating the presence and degree of ROR1 expression. Immunohistochemical analysis of tumor biopsies or surgical specimens may be preferred for this purpose. Methods for immunohistochemical analysis of tumor tissues are well known in the art.

Anti-ROR1 monoclonal antibodies useful in treating breast and other cancers include those that are capable of initiating a potent immune response against the tumor and those that are capable of direct cytotoxicity. In this regard, anti-ROR1 monoclonal antibodies (mAbs) may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, anti-ROR1 mAbs that exert a direct biological effect on tumor growth are useful in the practice of the invention. Potential mechanisms by which such directly cytotoxic mAbs may act include inhibition of cell growth, modulation of cellular differentiation, modulation of tumor angiogenesis factor profiles, and the induction of apoptosis. The mechanism by which a particular anti-ROR1 mAb exerts an anti-tumor effect may be evaluated using any number of in vitro assays designed to determine ADCC, ADMMC, complement-mediated cell lysis, and so forth, as is generally known in the art.

The use of murine or other non-human monoclonal antibodies, or human/mouse chimeric mAbs may induce moderate to strong immune responses in some patients. In some cases, this will result in clearance of the antibody from circulation and reduced efficacy. In the most severe cases, such an immune response may lead to the extensive formation of immune complexes which, potentially, can cause renal failure. Accordingly, some monoclonal antibodies used in the practice of the therapeutic methods of the invention are those that are either fully human or humanized and that bind specifically to the target ROR1 antigen with high affinity but exhibit low or no antigenicity in the patient.

Therapeutic methods of the invention contemplate the administration of single anti-ROR1 mAbs as well as combinations, or cocktails, of different mAbs (e.g. anti-ROR1 and anti-Her-2 antibodies). Such mAb cocktails may have certain advantages inasmuch as they contain mAbs that target different epitopes, exploit different effector mechanisms or combine directly cytotoxic mAbs with mAbs that rely on immune effector functionality. Such mAbs in combination may exhibit synergistic therapeutic effects. In addition, the administration of anti-ROR1 mAbs may be combined with other therapeutic agents, including but not limited to various chemotherapeutic agents, androgen-blockers, and immune modulators (e.g., IL-2, GM-CSF). The anti-ROR1 mAbs may be administered in their “naked” or unconjugated form, or may have therapeutic agents conjugated to them.

The anti-ROR1 antibody formulations may be administered via any route capable of delivering the antibodies to the tumor site. Potentially effective routes of administration include, but are not limited to, intravenous, intraperitoneal, intramuscular, intratumor, intradermal, and the like. Treatment will generally involve the repeated administration of the anti-ROR1 antibody preparation via an acceptable route of administration such as intravenous injection (IV), typically at a dose in the range of about 0.1 to about 10 mg/kg body weight. Doses in the range of 10-500 mg mAb per week may be effective and well tolerated.

Based on clinical experience with the Herceptin mAb in the treatment of metastatic breast cancer, an initial loading dose of approximately 4 mg/kg patient body weight IV followed by weekly doses of about 2 mg/kg IV of the anti-ROR1 in mAb preparation may represent an acceptable dosing regimen. Preferably, the initial loading dose is administered as a 90 minute or longer infusion. The periodic maintenance dose may be administered as a 30 minute or longer infusion, provided the initial dose was well tolerated. However, as one of skill in the art will understand, various factors will influence the ideal dose regimen in a particular case. Such factors may include, for example, the binding affinity and half life of the Ab or nabs used, the degree of ROR1 expression in the patient, the extent of circulating shed ROR1 antigen, the desired steady-state antibody concentration level, frequency of treatment, and the influence of chemotherapeutic agents used in combination with the treatment method of the invention.

### Inhibition of ROR1 Protein Function

The invention includes various methods and compositions for inhibiting the binding of ROR1 to its binding partner or ligand, or its association with other protein(s) as well as methods for inhibiting ROR1 function.

### Inhibition of ROR1 With Intracellular Antibodies

In one approach, recombinant vectors encoding single chain antibodies that specifically bind to ROR1 may be introduced into ROR1 expressing cells via gene transfer technologies, wherein the encoded single chain anti-ROR1 antibody is expressed intracellularly, binds to ROR1 protein, and thereby inhibits its function. Methods for engineering such intracellular single chain antibodies are well known. Such intracellular antibodies, also known as “intrabodies”, may be specifically targeted to a particular compartment within the cell, providing control over where the inhibitory activity of the treatment will be focused. This technology has been successfully applied in the art (for review, see Richardson and Marasco, 1995, TIBTECH vol. 13). Intrabodies have been shown to virtually eliminate the expression of otherwise abundant cell surface receptors. See, for example, Richardson et al., 1995, Proc. Natl. Acad. Sci. USA 92: 3137-3141; Beerli et al., 1994, J. Biol. Chem. 289: 23931-23936; Deshane et al., 1994, Gene Ther. 1: 332-337.

Single chain antibodies comprise the variable domains of the heavy and light chain joined by a flexible linker polypeptide, and are expressed as a single polypeptide. Optionally, single chain antibodies may be expressed as a single chain variable region fragment joined to the light chain constant region. Well known intracellular trafficking signals may be engineered into recombinant polynucleotide vectors encoding such single chain antibodies in order to precisely target the expressed intrabody to the desired intracellular compartment. For example, intrabodies targeted to the endoplastic reticulum (ER) may be engineered to incorporate a leader peptide and, optionally, a C-terminal ER retention signal, such as the KDEL amino acid motif. Intrabodies intended to exert activity in the nucleus may be engineered to include a nuclear localization signal. Lipid moieties may be joined to intrabodies in order to tether the intrabody to the cytosolic side of the plasma membrane. Intrabodies may also be targeted to exert function in the cytosol. For example, cytosolic intrabodies may be used to sequester factors within the cytosol, thereby preventing them from being transported to their natural cellular destination.

In one embodiment, intrabodies may be used to capture ROR1 in the nucleus, thereby preventing its activity within the nucleus. Nuclear targeting signals may be engineered into such ROR1 intrabodies in order to achieve the desired targeting. Such ROR1 intrabodies may be designed to bind specifically to a particular ROR1 domain. In another embodiment, cytosolic intrabodies that specifically bind to the ROR1 protein may be used to prevent ROR1 from gaining access to the nucleus, thereby preventing it from exerting any biological activity within the nucleus (e.g., preventing ROR1 from forming transcription complexes with other factors).

Inhibition of ROR1 with Recombinant Proteins

In another approach, recombinant molecules that are capable of binding to ROR1 or its binding partner(s) thereby preventing ROR1 from accessing/binding to its binding partner(s) or associating with other protein(s) are used to inhibit ROR1 function. For example, the recombinant molecule can include the extracellular domain of ROR1 or a portion thereof, such as the Ig loop domain of ROR1, the frizzled domain of ROR1 or the kringle domain of ROR1. In some embodiments of the invention, the recombinant molecules includes 2 or alternatively 3 of these ROR1 domains.

Alternatively, such recombinant molecules may, for example, contain the reactive part(s) of a ROR1 specific antibody molecule. In a particular embodiment, the ROR1 binding domain of a ROR1 binding partner may be engineered into a dimeric fusion protein comprising two ROR1 ligand binding domains linked to the Fc portion of a human IgG, such as human IgG1. Such IgG portion may contain, for example, the C_{H}2 and C_{H}3 domains and the hinge region, but not the C_{H}1 domain. Such dimeric fusion proteins may be administered in soluble form to patients suffering from a cancer associated with the expression of ROR1, including but not limited to breast cancers, where the dimeric fusion protein specifically binds to ROR1 thereby blocking ROR1 interaction with a binding partner. Such dimeric fusion proteins may be further combined into multimeric proteins using known antibody linking technologies.

### Inhibition of ROR1 Transcription or Translation

Within another class of therapeutic approaches, the invention provides various methods and compositions for inhibiting the transcription of the ROR1 gene. Similarly, the invention also provides methods and compositions for inhibiting the translation of ROR1 mRNA into protein.

In one approach, a method of inhibiting the transcription of the ROR1 gene comprises contacting the ROR1 gene with a ROR1 antisense polynucleotide. In another approach, a method of inhibiting ROR1 mRNA translation comprises contacting the ROR1 mRNA with an antisense polynucleotide. In another approach, a ROR1 specific ribozyme may be used to cleave the ROR1 message, thereby inhibiting translation. Such antisense and ribozyme based methods may also be directed to the regulatory regions of the ROR1 gene, such as the ROR1 promoter and/or enhancer elements. Similarly, proteins capable of inhibiting a ROR1 gene transcription factor may be used to inhibit ROR1 mRNA transcription. The various polynucleotides and compositions useful in the aforementioned methods have been described above. The use of antisense and ribozyme molecules to inhibit transcription and translation is well known in the art.

Other factors that inhibit the transcription of ROR1 through interfering with ROR1 transcriptional activation may also be useful for the treatment of cancers expressing ROR1. Similarly, factors that are capable of interfering with ROR1 processing may be useful for the treatment of cancers expressing ROR1. Cancer treatment methods utilizing such factors are also within the scope of the invention.

### General Considerations for Therapeutic Strategies

Gene transfer and gene therapy technologies may be used for delivering therapeutic polynucleotide molecules to tumor cells synthesizing ROR1 (i.e., antisense, ribozyme, polynucleotides encoding intrabodies and other ROR1 inhibitory molecules). A number of gene therapy approaches are known in the art. Recombinant vectors encoding ROR1 antisense polynucleotides, ribozymes, factors capable of interfering with ROR1 transcription, and so forth, may be delivered to target tumor cells using such gene therapy approaches.

The above therapeutic approaches may be combined with any one of a wide variety of chemotherapy or radiation therapy regimens. These therapeutic approaches may also enable the use of reduced dosages of chemotherapy and/or less frequent administration, particularly in patients that do not tolerate the toxicity of the chemotherapeutic agent well.

The anti-tumor activity of a particular composition (e.g., antisense, ribozyme, intrabody), or a combination of such compositions, may be evaluated using various in vitro and in vivo assay systems. In vitro assays for evaluating therapeutic potential include cell growth assays, soft agar assays and other assays indicative of tumor promoting activity, binding assays capable of determining the extent to which a therapeutic composition will inhibit the binding of ROR1 to a binding partner, etc.

In vivo, the effect of a ROR1 therapeutic composition may be evaluated in a suitable animal model. For example, xenogenic breast cancer models wherein human breast cancer explants or passaged xenograft tissues are introduced into immune compromised animals, such as nude or SCID mice, are appropriate in relation to breast cancer and have been described in the art. Efficacy may be predicted using assays that measure inhibition of tumor formation, tumor regression or metastasis, and the like.

In vivo assays that qualify the promotion of apoptosis may also be useful in evaluating potential therapeutic compositions. In one embodiment, xenografts from beating mice treated with the therapeutic composition may be examined for the presence of apoptotic foci and compared to untreated control xenograft-bearing mice. The extent to which apoptotic foci are found in the tumors of the treated mice provides an indication of the therapeutic efficacy of the composition.

The therapeutic compositions used in the practice of the foregoing methods may be formulated into pharmaceutical compositions comprising a carrier suitable for the desired delivery method. Suitable carriers include any material that when combined with the therapeutic composition retains the anti-tumor function of the therapeutic composition and is non-reactive with the patient's immune system. Examples include, but are not limited to, any of a number of standard pharmaceutical carriers such as sterile phosphate buffeted saline solutions, bacteriostatic water, and the like (see, generally, Remington's Pharmaceutical Sciences 16th Ed., A. Osal., Ed., 1980).

Therapeutic formulations may be solubilized and adminstered via any route capable of delivering the therapeutic composition to the tumor site. Potentially effective routes of administration include, but are not limited to, intravenous, parenteral, intraperitoneal, intramuscular, intratumor, intradermal, intraorgan, orthotopic, and the like. A common formulation for intravenous injection comprises the therapeutic composition in a solution of preserved bacteriostatic water, sterile unpreserved water, and/or diluted in polyvinylchloride or polyethylene bags containing 0.9% sterile Sodium Chloride for Injection, USP. Therapeutic protein preparations may be lyophilized and stored as sterile powders, preferably under vacuum, and then reconstituted in bacteriostatic water containing, for example, benzyl alcohol preservative, or in sterile water prior to injection.

Dosages and administration protocols for the treatment of cancers using the foregoing methods will vary with the method and the target cancer and will generally depend on a number of other factors appreciated in the art.

### Kits

For use in the diagnostic and therapeutic applications described or suggested above, kits are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a ROR1 protein or a ROR1 gene of message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

A typical embodiment of the invention is a kit comprising a container, a label on said container, and a composition contained within said container; wherein the composition includes a ROR1 specific antibody and/or a polynucleotide that hybridizes to a complement of the ROR1 polynucleotide shown in SEQ ID NO: 1 under stringent conditions (or binds to a ROR1 polypeptide encoded by the polynucleotide shown in SEQ ID NO: 1), the label on said container indicates that the composition can be used to evaluate the presence of ROR1 protein, RNA or DNA in at least one type of mammalian cell, and instructions for using the ROR1 antibody and/or polynucleotide for evaluating the presence of ROR1 protein, RNA or DNA in at least one type of mammalian cell.

The kit of the invention will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

### Methods for Discovering Genes Such as ROR1

The disclosure also provides optimized methods of data mining including those used to identify ROR1 as a gene of diagnostic significance. These methodologies include novel experimental analyses as well as constraint-based public data analyses. These methods of the invention include a number of discreet actions or steps that can occur in a wide variety of sequential orders. These steps are then combined to identify genes of interest such as ROR1. In a preliminary step, an artisan can define a working gene set, for example from experimentally generated gene lists and/or a literature based gene selection. In another step artisans can undertake microarray screens of gene expression in for example, +/− HER-2 cell lines, +/− ligands/antagonists, primary breast cancers, breast cancer cell lines or the like. In another step, artisans can employ candidate selection parameter to identify genes of interest, for example a focus on genes that can be grouped into signaling pathways that are likely to contribute to the progression of breast cancer (e.g. RTKs (receptor tyrosine kinases)). In another step, the artisan can evaluate and/or confirm the expression of gene(s) of interest via well-known protocols such as quantitative PCR, northerns, and western analyses. In another step, the artisan can develop and test a hypothesis based on the results of the prior steps, for example a hypothesis correlating ROR1 expression with one or more breast cancer subtypes and/or with a poor prognosis. In this step, artisans can consider factors such as whether a functional significance of expression patters are measurable using bioassays and cell line models. For example, one can use human tumor tissues to further evaluate differential expression etc. and use xenograft models to confirm the functional relevance of the observations in vivo.

In one such illustrative data mining method, an initial observation can come from constraints-based analysis of public expression data and cell line data to, for example, identify interesting characteristics of a gene such as ROR1. Using this first observation, one can then develop a hypothesis correlating a breast cancer subtype with poor prognosis and ROR1 (a potential molecular target). One can then validate ROR1 overexpression in relevant breast cancer cell lines and tumors. One can then generate experimental data supporting biological functions of ROR1 in breast cancer pathogenesis.

In an illustrative embodiment, the initial observation can be from constraints-based analysis of public expression data. In this embodiment, one can select a working gene set comprising receptor tyrosine kinases and their ligands. One can then work to integrate this selection with other studies known in the art, for example by integrating the disclosure in Van't Veet, L. J., et al. (2002) Nature 415, 530-536 (“Rosetta/Netherlands”) with that in Sorlie et al., Proc Natl Acad Sci USA. 2001 Sep. 11; 98(19):10869-74. Briefly, Van't Veer et al. (2002) Nature 415, 530-536 notes that breast cancer patients with the same stage of disease can have markedly different treatment responses and overall outcome. In this study Van't Veer et al. used DNA microarray analysis on primary breast tumours of 117 young patients, and applied supervised classification to identify a gene expression signature strongly predictive of a short interval to distant metastases (“poor prognosis” signature) in patients without tumor cells in local lymph nodes at diagnosis (lymph node negative). In this way they establish a signature that identifies tumors of, for example, BRCA1 carriers and teach that this gene expression profile will outperform all currently used clinical parameters in predicting disease outcome. Van't Veer et al. teach that a three step supervised clustering of 78 sporadic tumors based on strength of correlation coefficient with prognosis identifies a subset of 70 genes from 5000 differentially expressed genes that predict distant metastasis within 5 years with 83% accuracy.

Similarly, the Sorlie et al., Proc Natl Acad Sci USA. 2001 Sep. 11; 98(19):10869-74 Stanford/Norway study also classifies breast carcinomas based on variations in gene expression patterns derived from cDNA microarrays and to correlate tumor characteristics to clinical outcome. This article identifies a number of subtypes of breast carcinoma that are associated with significantly different clinical outcomes. The subtypes of breast carcinoma include basal-like, ERBB2+, and luminal subtypes A and B (see, e.g. FIG. 1 in Sorlie et al. supra).

One can employ clustering algorithms that analyze coordinate gene expression patterns as part of a classifications prognosis. This analysis also allows the identification of therapeutic targets. In some embodiments of the invention, this step can include a pathogenesis constraints based hypothesis building where one can focus on genes and pathways likely to be important for disease progression such as those involved in (or having domain with homology to proteins know to be involved in) in disease, growth disregulation, cell cycling and the like. In such constraints based methods for target identification using gene expression profiles one can consider a number of factors such as the observation that breast cancer is heterogeneous, that prognostic markers and molecules have already been shown to be important for subtypes of breast cancers (i.e. ER, HER-2), and that it is unlikely that the same set of genes will be “prognostic” or serve as appropriate therapeutic targets in all breast cancers.

In an illustrative embodiment of this methodology, one can for example select a data set for analysis (e.g. some number of genes), optionally selected from sporadic and/or heritable cancers (e.g. BRCA 1 and or 2 tumors). One can then focus on a set of genes for analysis such as breast cancer related genes (e.g. those in known databases such as omim, breast cancer database, ncbi), Stanford tumor type markers, ERBB2 regulated genes from cell line data, chemokines and receptor tyrosine kinases and ligands, epithelial junction proteins and the like. One can then classify samples according to certain gene (e.g. ERBB2 and ESR1 etc.) expression levels and/or BRCA1 mutation status etc. to identify a working gene set. For example Wilson et al., Breast Cancer Research Vol 6 No. 5: 192-200 (2004) (which is incorporated by reference) teach that estrogen receptor 1 expression and HER amplification can be used to define breast cancer subtypes.

Following these steps, one can then delineate groups with no overlapping samples that are for example, roughly equivalent the Stanford/Norway classifications discussed above. In one embodiment, sporadic tumor samples can first classified on the basis of their HER2 expression and the remaining samples can be grouped by ESR1 expression. The sporadic tumor categories can be non-overlapping, since no HER2+ sample had an ESR1 ratio>0, Samples with a BRCA mutation can be classified separately. In such a grouping, all of the BRCA tumors are shown to have ESR1<0 and HER2<0. The HER2+ and ESR1⁻⁻ tumors exhibit the poorest prognosis, followed by ESR1++.

In some embodiments of the analysis of this working gene set one can employ “bin” data rather than “cluster” data and can for example build matrices to quantify the frequency of up-regulated and downregulated genes across sample and by group. Optionally one can investigate co-expression of members of working gene set across tumor groups. One can also generate hypotheses regarding pathogenesis by tumor group. In this way, one can identify potential targets and test for statistical significance. An exemplary working set includes known breast cancer genes, Stanford tumor type markets, ERBB2 regulated genes, chemokines/RTK and ligands, and/or epithelial junction proteins. For bin data, one can then create data matrices, for example: level 1: ratio for each gene/sample; level 2: binary value each gene/sample; level 3: total up or down by gene/group; level 4: co-expression gene family/group. Optionally, one can focus on receptor tyrosine kinases, with the working gene set included all RTKs and their ligands that were available in for example, the Rosetta/Netherlands data (147 elements representing 127 out of 130 possible unique RTKs and their ligands). One can then identify tumor group-specific RTK/ligand expression.

Embodiments of this methodology were used in the identification of ROR1 as a gene of interest. ROR1 is a receptor tyrosine kinase specifically up-regulated in basal and BRCA1 tumors. FIG. B shows ROR1 mRNA expression in Rosetta/Netherlands data. ROR1 is a novel family of cell surface receptors with tyrosine kinase-like domain (see, Masiakowski et al., JBC, 267 26181-26190 (1992). While the ligand(s) for ROR1/2 are not known, the presence of a CRD (cysteine-rich domain) or frizzled domain suggests that RORs may bind WNTs.

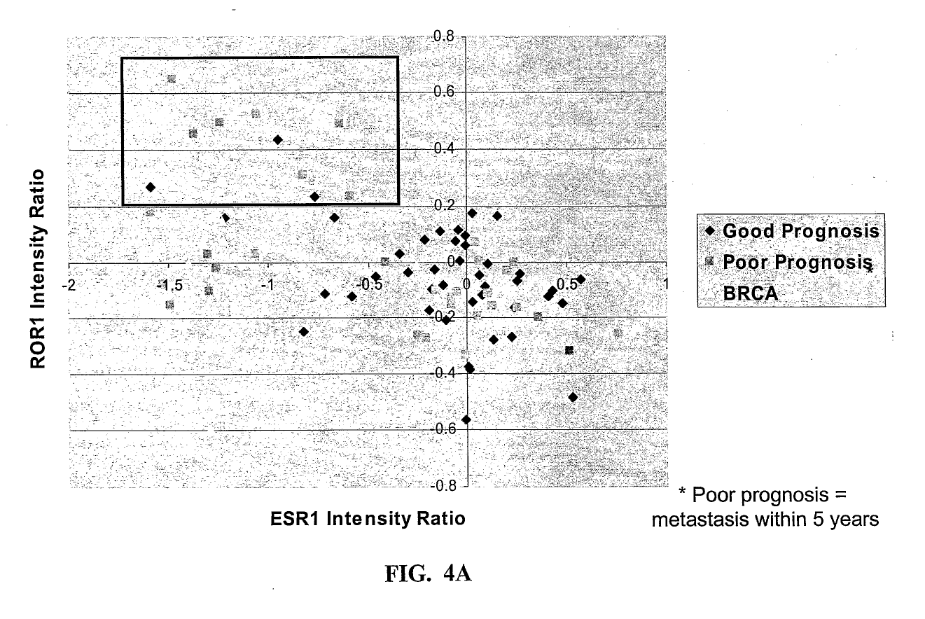

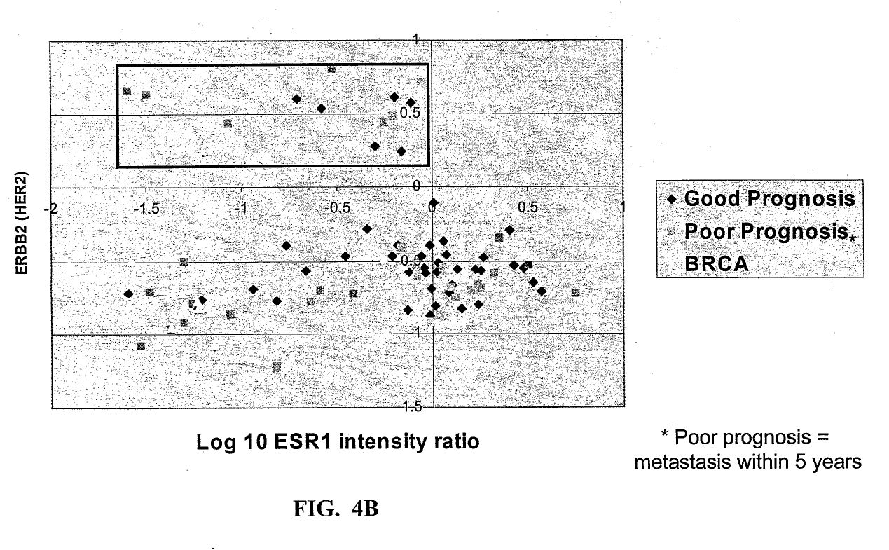

As disclosed herein, these methods allow the development of a hypothesis of ROR1 biology as well as the design of tests for correlating ROR1 expression with prognosis, and/or breast cancer subtype and the like. For example, using this approach we find that basal and BRCA1 breast cancers are related by cellular origin and molecular pathogenesis and that the over-expression of ROR1 is an important alteration that is involved in the pathogenesis of these two tumor groups. As shown in FIG. 4A, ROR1 overexpressing tumors are associated with a poor prognosis in the Rosetta/Netherlands tumors. The percentage (70% of sporadic) of poor prognosis tumors in the ROR1 group is higher than that for any other single prognostic gene analyzed including HER-2, EGFR, V-EGF, FLT3, myc, UPA and PAI. As shown in FIG. 4B, this finding is analogous to that observed with HER-2, where fifty-four percent of HER-2 overexpressing tumors are poor prognosis samples

The significance of ROR1 overexpression in relevant breast cancer cell lines and tumors can be further validated in a number of ways. The ROR1 gene is located at position 1p31.3. In addition, ROR1 over-expressing cell lines have basal or mesenchymal characteristics. Another element AK000776 which is just distal to ROR1 is also present on DNA microarrays such as the Rosetta chip. ROR1 and AK000776 show a strong positive linear correlation. The Northern Blot Analysis in FIG. 5A shows ROR1 mRNA expression in a number of breast cancer cell lines. This data confirms the ROR1 expression observed in Groups 4 and 6 of Rosetta Tumor Data.

The identification of ROR1 as a gene of interest and the subsequent validation of this observation demonstrate the power of the data mining methods disclosed above.

### EXAMPLES

Various aspects of the invention are further described and illustrated by way of the several examples that follow, none of which are intended to limit the scope of the invention.

### Example 1

### Production of Recombinant ROR1 in a Mammalian System

To express recombinant ROR1, the full length ROR1 cDNA can be cloned into an expression vector known in the art such as one that provides a 6H is tag at the carboxyl-terminus (pCDNA 3.1 myc-his, Invitrogen). The constructs can be transfected into an appropriate cell such as MCF-7 cells. The ROR1 genes can also be subcloned into a retroviral expression vector such as pSRαMSV tkneo and used to establish ROR1 expressing cell lines as follows. The ROR1 coding sequence (from translation initiation ATG to the termination codons) can be amplified by PCR using ds cDNA template from ROR1 cDNA. The PCR product is subcloned into pSRαMSVtkdeo via the EcoR1 (blunt-ended) and Xba 1 restriction sites on the vector and transformed into DH5α competent cells. Colonies are picked to screen for clones with unique internal restriction sites on the cDNA. The positive clone is confirmed by sequencing of the cDNA insert. Retroviruses may thereafter be used for infection and generation of various cell lines using, for example, NIH 3T3, TsuPr1, MCF-7 or rat-1 cells.

### Example 2

### Generation of ROR1 Polyclonal and Monoclonal Antibodies

Polyclonal antibodies can be raised in a mammal such as a rabbit, for example, by one or more injections of an immunizing agent and, if desired, an adjuvant. Typically, the immunizing agent and/or adjuvant will be injected in the mammal by multiple subcutaneous or intraperitoneal injections. Typically an immunizing agent may include all or portions of die ROR protein, or fusion proteins thereof.

For example, a portion of ROR1 comprising the Ig C2 like and frizzled domains (termed “IF”) was cloned into the vector pET32A (Novagen) and expressed as a Thio/HIS fusion protein. This protein construct is highly expressed in insoluble inclusion bodies. Upon being solubilized with 6M urea, the fusion protein binds Ni columns efficiently under denaturing conditions. Rabbits were then immunized with this fusion protein and subsequently bled in order to generate polyclonal sera. FIG. 5F and FIG. 5G show the detection of endogenous ROR1 protein in CAL51 cells using this rabbit polyclonal sera, with SKBR cells serving as a comparative cancer cell.

Like polyclonal antibodies, monoclonal antibodies can be generated by well known methods in the art. In order to generate ROR1 monoclonal antibodies for example, a fusion protein (e.g. glutathione s transferase) encompassing a ROR1 protein can be synthesized and used as immunogen. In another example of a method for generating ROR1 antibodies, an immunogen is prepared which consists of a HIS tagged ROR domain such as the frizzled domain. This construct can be inserted into a baculovirus vector which is then introduced into insect cells in a manner that allows the a native (folded) immunogenic protein to be secreted into the media. Optionally immunogens can be conjugated to a second protein known to stimulate the immune response such as ICLH prior to immunization. Alternatively, ROR1 IF immunogen construct can be made in bacteria. In situations where the immunogenic protein is insoluble, it can be optionally denatured with Urea prior to immunization. Alternatively, a ROR1 complete ECD immunogen construct can be made as part of a Ig fusion construct and then expressed in mammalian cells (e.g. CHO cells) and purified using the Ig portion fusion construct prior to immunization.

In an illustrative embodiment, mice can be initially immunized (e.g. intraperitoneally) with an appropriate amount of an immunogen comprising the FRZ domain of ROR1. Optionally the immunogen can be conjugated to KLH, and/or mixed in complete Freund's adjuvant. Mice can be subsequently immunized (e.g. every 2 weeks with this ROR1 immunogen), optionally mixed in Freund's incomplete adjuvant. Reactivity of serum from immunized mice can be monitored by ELISA using this ROR1 immunogen. Mice showing the strongest reactivity can be rested and given a final injection of immunogen and then sacrificed. The spleens of the sacrificed mice can then be harvested and fused to SPO/2 myeloma cells using standard procedures. Supernatants from growth wells following HAT selection are typically screened by ELISA and western blot to identify ROR1 specific antibody producing clones.

The binding affinity of a ROR1 monoclonal antibody can be determined using standard technology. Affinity measurements quantify the strength of antibody to epitope binding and may be used to help define which ROR1 monoclonal antibodies are preferred for diagnostic or therapeutic use. The BIAcore system (Uppsala, Sweden) is a common method for determining binding affinity. The BIAcore system uses surface plasmon resonance (SPR, Welford, K., 1991, Opt. Quant. Elect. 23:1; Morton and Myszka, 1998, Methods in Enzymology 295:268) to monitor biomolecular interactions in real time. BIAcore analysis conveniently generates association late constants, dissociation rate constants, equilibrium dissociation constants, and affinity constants.

### Example 3

### RT-PCR Expression Analysis

A variety of PCR protocols for analyzing ROR1 expression in a cell are well known in the art. The following provides an illustration of one typical protocol.

First strand cDNAs can be generated from a sufficient amount (e.g. 1 μg) of mRNA with a primer such as oligo (dT)12-18 priming using a commercially available system such as the Gibco-BRL Superscript Preamplification system. The manufacturer's protocol can be used. These typically include an incubation for 50 min at 42° C. with reverse transcriptase followed by RNAse H treatment at 37° C. for 20 min. After completing the reaction, the volume can be increased with water prior to normalization. Normalization of the first strand cDNAs from normal and cancer tissues can be performed by using primers to a housekeeping gene such as β-actin. For example, first strand cDNA (5 μl) can be amplified in a total volume of 50 μl containing 0.4 μM primers, 0.2 μM each dNTPs, 1×PCR buffer (Gibco-BRL, 10 mM Tris-HCL, 1.5 mM MgCl₂, 50 mM KCl, pH8.3) and 1× Platinum Taq DNA polymerase (Gibco-BRL). PCR can be performed using an thermal cycler under the following conditions: Initial denaturation can be at 94° C. for 45 sec, followed by a 18, 20, and 22 cycles of 94° C. for 45, 58° C. for 45 sec, 72° C. for 45 sec. A final extension at 72° C. can be carried out for 2 min. Five μl of the PCR reaction can be removed at 18, 20, and 22 cycles and used for agarose gel electrophoresis. After agarose gel electrophoresis, the band intensities of the 283 b.p. β-actin bands from multiple tissues can be compared by visual inspection. Dilution factors for the first strand cDNAs can be calculated to result in equal β-actin band intensities in all tissues after 22 cycles of PCR. Three rounds of normalization can be required to achieve equal band intensities in all tissues after 22 cycles of PCR. To determine expression levels of the ROR1 gene, 5 μl of normalized first strand cDNA can be analyzed by PCR using 26, and 30 cycles of amplification. Quantitative expression analysis can be achieved by comparing the PCR products at cycle numbers that give light band intensities. RT-PCR expression analysis can be performed on first strand cDNAs generated using pools of tissues from multiple normal and cancer samples. The cDNA normalization can be demonstrated in every experiment using a housekeeping gene such as beta-actin.

### Example 4

### Examining the Role of ROR1 in Basal, ER-negative Breast Cancer

Immunohistochemical and mRNA expression profiling studies of large breast cancer cohorts have reproducibly identified a subset of tumors that express markers, such cytokeratin 5, that are characteristic of the basal layer of the mammary gland (see, e.g. Sorlie et al., Proc NetAcad Sci USA. 2003; 100: 8418-23; Sorlie et al., Prop Natl Acad Sci USA. 2001; 98: 10869-74; and Foulkes et al., J Natl Cancer Inst. 2003; 95: 1482-5). It has been suggested that these malignancies arise from basal or supra-basal progenitor cells with stem cell attributes. This is in contrast to many human breast cancers that uniformly express the simple glandular cytokeratins (K8/18/19) suggesting their origins as transformed luminal epithelial cells. Human breast cancers with basal features are invariably estrogen receptor (ER) negative, rarely contain amplified HER-2, are generally high grade/poorly differentiated and are associated with poor prognosis (see, e.g. Sorlie et al., Proc Natl Acad Sci USA. 2003; 100: 8418-23; Sorlie et al., Proc Natl Acad Sci USA. 2001; 98: 10869-74; and Foulkes et al., J Natl Cancer Inst. 2003; 95: 1482-5).

Although high frequencies of p53 mutations have been associated with basal cancers and tumors arising in BRCA1 carriers fall into to this basal class (see, e.g. Sorlie et al., Proc Natl Acad Sci USA 2003; 100: 8418-23; Sorlie et al., Proc Natl Acad Sci USA. 2001; 98: 10869-74; and Foulkes et al., J Natl Cancer Inst. 2003; 95: 1482-5), the oncogenic molecules and key molecular pathways that drive the progression of these tumors are unknown. As disclosed herein, using microarray profiling and Northern blot confirmation we have demonstrated that the ROR1 receptor tyrosine kinase is highly expressed in primary human breast cancers with an ER negative, basal phenotype. We have also found high ROR1 expression in several human breast cancer cell lines that co-express basal markers, while ROR1 expression was not detected in any luminal cell lines. Importantly, the level of ROR1 expression detected in basal, malignant cell lines is significantly higher than in non-malignant cells. An additional feature of ROR1 is that it may bind wnt ligands via an extracellular frizzled domain thus providing a possible link to a signaling pathway previously shown to regulate progenitor cells (see, e.g. Saldanha et al., Protein Sci. 1998; 7: 1632-5).

The disclosure provided herein allows those of skill in the art to identify candidate genes that drive the progression of these poorly understood basal, ER negative human breast cancers. While not being bound by a specific scientific theory, the highly suggestive expression pattern of ROR1 in combination with the established importance of receptor tyrosine kinases (e.g. HER2, EGFR, VEGFR) in tumor formation prompted us to propose the hypothesis that ROR1 plays a critical role in the pathogenesis of basal tumors. The oncogenic potential of ROR1 has not previously been explored.

A first set of experiments test the hypothesis that ROR1 preferentially transforms basal/progenitor cells of the mouse mammary gland.

Determining if inducible over-expression of ROR1 can transform mouse mammary epithelial cells.

Transgenic, conditional TetO-ROR1 mice can be generated and crossed to existing MMTV-rtTA mice (see, e.g. Gunther et al., FASEB J. 2002; 16: 283-92) to achieve doxycycline-dependent (tet-on) expression of ROR1 specifically in the mammary gland.

Determining if ROR1 over-expression preferentially transforms the basal/progenitor cell lineages of the mammary gland.

These TetO-ROR1 mice will then be crossed to strains expressing rtTA under the control of the keratin 5 (K5) promoter to drive expression specifically in the basal/progenitor compartments of the mammary gland and other tissues.

Illustrative Methods:

Transgene expression in MMTV-rtTA/TetO-ROR1 and K5-rtTA/TetO-ROR1 mice can be induced with doxycycline beginning at 6 weeks of age. Expression of ROR1 can be examined by in situ hybridization, northern blotting and immunohistochemistry. Changes in tissue architecture and the presence of pre-malignant or malignant lesions can be assessed at increasing intervals following transgene induction by the analysis of carmine-stained mammary whole mounts and hematoxylin & eosin stained tissue sections. The cellular origin of any hyperplastic lesions or overt carcinomas can be investigated using immunohistochemical staining with intermediate filament markers, adhesion proteins and putative stem cell makers (K8/K18/K19 for luminal cells, K5/K6/K14/P-cadherin/Sca-1 for basal/progenitor cells). As a backup, K14-rtTa mice can be considered to drive ROR1 expression.

Relevance:

Human breast cancers with basal properties are aggressive malignancies that are not responsive to established targeted therapies such as anti-estrogens or Herceptin since they are invariably ER negative and rarely contain amplified HER-2. The ROR1 cell surface receptor is a tractable therapeutic target accessible by monoclonal antibodies or small molecule tyrosine kinase inhibitors. The demonstration that ROR1 over-expression drives basal breast cancers in the mouse provides a rationale for the development of ROR1 targeted therapeutics that specifically treat basal breast cancers.

### Example 5

### A Novel Receptor Tyrosine Kinase and the Control of Multipotent Mammary Progenitor Cells

Human estrogen receptor (ER) positive tumors and mouse mammary tumors induced by oncogenic Neu or H-Rat express cell type markets consistent with a differentiated luminal origin (e.g. cytokeratins K18/K19). In contrast, aggressive ER-negative human cancers and murine tumors induced by the Wnt-1 oncogene, display a much more heterogeneous pattern of cell type markers including the basal cytokeratins K5, K17, K14, and stem cell antigen (Sca1) (see, e.g. Li et al., Proc Natl Acad Sci USA. 2003; 100: 15853-8). This is consistent with the idea that multipotent progenitor cells are the targets of transformation in these breast cancers. Immortalized progenitor cells have been described that are capable of differentiating into both luminal and myoepithelial lineages (see, e.g. Gudjonsson et al., Genes Dev. 2002; 16: 693-706; and Deugnier et al., J Cell Biol. 2002; 159: 453-63).

Although most human breast cancer cell lines express homogeneous luminal markers, we have recently identified multiple malignant breast cell lines that appear to have progenitor properties in that they produce both K18/K19 and smooth muscle actin (SMA) positive cells. Strikingly, we have discovered that both the non-malignant and the cancer lines with progenitor properties consistently express the ROR1 receptor tyrosine kinase while luminal mammary cells have no detectable expression. We also found high-level expression of ROR1 in a subset of primary human breast cancers with basal/progenitor properties. Additionally, ROR1 may bind Wnt ligands via its extracellular frizzled domain thus providing a link to a signaling pathway that is known to regulate progenitor cells (see, e.g. Saldanha et al., Protein Sci. 1998; 7: 1632-5; and Brittan et al., J Pathol. 2002; 197: 492-509).

The highly suggestive ROR1 expression pattern combined with the intriguing possibility that it may bind Wnt ligands which hive established roles as critical mediators of stem cell renewal, led us to hypothesize that ROR1 signaling participates in the control of mammary progenitor cell proliferation and/or self renewal. We further hypothesize that since malignant cells with similar progenitor properties have even higher levels of ROR1, cells may up-regulate this pathway during malignant progression. The disclosure provided herein allows one to test the hypothesis that signaling through the ROR1 receptor tyrosine kinase controls the proliferation, self-renewal and/or differentiation of multipotent mammary progenitor cells.

Determine ROR1 silencing in mammary cells with progenitor properties critically affects their proliferation, morphogenesis and/or differentiation capacity.

ROR1 expression can be silenced by RNA interference in non-malignant and malignant cells with progenitor properties and the effects assayed in morphogenic and tumorigenic assays.

Determine if increased signaling from the ROR1 receptor specifically transforms or increases the malignancy of mammary epithelial basal/progenitor cells compared to luminal breast cells.

The effects of ROR1 over-expression or constitutively activation on the proliferation and malignant potential of basal/progenitor and luminal cell lines can be compared.

### Illustrative Methods:

Silencing of ROR1 can be accomplished by stable of expression hpRNA ROR1 sequences using the pSIREN-retroQ retroviral system (BD Clontech). Overexpression of ROR1 constructs including wild type, constitutively activated and deletion mutants missing the CRD or kinase domain can be achieved using retroviral infection (pLPCX; BD Clontech) can be monitored by using recently generated ROR1 polyclonal antibodies. The effects of depleted or overexpressed ROR1 can be assayed using in ratio matrigel TDLU formation assays (human cells), in vivo mammary epithelial reconstitution assays in cleared mammary fat pads (mouse cells) and in vivo xenograft tumor formation (malignant cells) as well as standard proliferation assays. The differentiation or cell type composition can be assessed by immunohistochemical staining using signature markets that regulate the growth and differentiation of mammary stem cells are not well understood and they may be critical for the pathogenesis of a particular class of aggressive ER-negative, basal breast cancers. Evidence that signaling through the ROR1 receptor tyrosine kinase controls the growth of mammary progenitor cells and the malignant cells derived from them could help explain how murine Wnt-1 induced tumors arise. Moreover, the properties of ROR1 as both cell surface receptor and a tyrosine kinase make it a particularly attractive therapeutic target.

Throughout this application, various publications are referenced (e.g. within parentheses). The disclosures of these publications awe hereby incorporated by reference herein in their entireties. Certain methods and materials in this application are analogous to those found in U.S. Pat. Nos. 6,767,541, 6,165,464, 5,772,997, 5,677,171, 5,770,195, 6,399,063, 5,725,856 and 5,720,954, the contents of which are incorporated herein by reference.

The present invention is not to be limited in scope by die embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### TABLES

TABLE 1
POLYNUCLEOTIDE SEQUENCES

HUMAN HER2 Polynucleotide Sequence (SEQ ID NO: 3)
ATGGAGCTGGCGGCCTTGTGCCGCTGGGGGCTCCTCCTCGCCCTCTTGCC
CCCCGGAGCCGCGAGCACCCAAGTGTGCACCGGCACAGACATGAAGCTGC
GGCTCCCTGCCAGTCCCGAGACCCACCTGGACATGCTCCGCCACCTCTAC
CAGGGCTGCCAGGTGGTGCAGGGAAACCTGGAACTCACCTACCTGCCCAC
CAATGCCAGCCTGTCCTTCCTGCAGGATATCCAGGAGGTGCAGGGCTACG
TGCTCATCGCTCACAACCAAGTGAGGCAGGTCCCACTGCAGAGGCTGCGG
ATTGTGCGAGGCACCCAGCTCTTTGAGGACAACTATGCCCTGGCCGTGCT
AGACAATGGAGACCCGCTGAACAATACCACCCCTGTCACAGGGGCCTCCC
CAGGAGGCCTGCGGGAGCTGCAGCTTCGAAGCCTCACAGAGATCTTGAAA
GGAGGGGTCTTGATCCAGCGGAACCCCCAGCTCTGCTACCAGGACACGAT
TTTGTGGAAGGACATCTTCCACAAGAACAACCAGCTGGCTCTCACACTGA
TAGACACCAACCGCTCTCGGGCCTGCCACCCCTGTTCTCCGATGTGTAAG
GGCTCCCGCTGCTGGGGAGAGAGTTCTGAGGATTGTCAGAGCCTGACGCG
CACTGTCTGTGCCGGTGGCTGTGCCCGCTGCAAGGGGCCACTGCCCACTG
ACTGCTGCCATGAGCAGTGTGCTGCCGGCTGCACGGGCCCCAAGCACTCT
GACTGCCTGGCCTGCCTCCACTTCAACCACAGTGGCATCTGTGAGCTGCA
CTGCCCAGCCCTGGTCACCTACAACACAGACACGTTTGAGTCCATGCCCA
ATCCCGAGGGCGGGTATACATTCGGCGCCAGCTGTGTGACTGCCTGTCCC
TACAACTACCTTTCTACGGACGTGGGATCCTGCACCCTCGTCTGCCCCCT
GCACAACCAAGAGGTGACAGCAGAGGATGGAACACAGCGGTGTGAGAAGT
GCAGCAAGCCCTGTGCCCGAGTGTGCTATGGTCTGGGCATGGAGCACTTG
CGAGAGGTGAGGGCAGTTACCAGTGCCAATATCCAGGAGTTTGCTGGCTG
CAAGAAGATCTTTGGGAGCCTGGCATTTCTGCCGGAGAGCTTTGATGGGG
ACCCAGCCTCCAACACTGCCCCGCTCCAGCCAGAGCAGCTCCAAGTGTTT
GAGACTCTGGAAGAGATCACAGGTTACCTATACATCTCAGCATGGCCGGA
CAGCCTGCCTGACCTCAGCGTCTTCCAGAACCTGCAAGTAATCCGGGGAC
GAATTCTGCACAATGGCGCCTACTCGCTGACCCTGCAAGGGCTGGGCATC
AGCTGGCTGGGGCTGCGCTCACTGAGGGAACTGGGCAGTGGACTGGCCCT
CATCCACCATAACACCCACCTCTGCTTCGTGCACACGGTGCCCTGGGACC
AGCTCTTTCGGAACCCGCACCAAGCTCTGCTCCACACTGCCAACCGGCCA
GAGGACGAGTGTGTGGGCGAGGGCCTGGCCTGCCACCAGCTGTGCGCCCG
AGGGCACTGCTGGGGTCCAGGGCCCACCCAGTGTGTCAACTGCAGCCAGT
TCCTTCGGGGCCAGGAGTGCGTGGAGGAATGCCGAGTACTGCAGGGGCTC
CCCAGGGAGTATGTGAATGCCAGGCACTGTTTGCCGTGCCACCCTGAGTG
TCAGCCCCAGAATGGCTCAGTGACCTGTTTTGGACCGGAGGCTGACCAGT
GTGTGGCCTGTGCCCACTATAAGGACCCTCCCTTCTGCGTGGCCCGCTGC
CCCAGCGGTGTGAAACCTGACCTCTCCTACATGCCCATCTGGAAGTTTCC
AGATGAGGAGGGCGCATGCCAGCCTTGCCCCATCAACTGCACCCACTCCT
GTGTGGACCTGGATGACAAGGGCTGCCCCGCCGAGCAGAGAGCCAGCCCT
CTGACGTCCATCGTCTCTGCGGTGGTTGGCATTCTGCTGGTCGTGGTCTT
GGGGGTGGTCTTTGGGATCCTCATCAAGCGACGGCAGCAGAAGATCCGGA
AGTACACGATGCGGAGACTGCTGCAGGAAACGGAGCTGGTGGAGCCGCTG
ACACCTAGCGGAGCGATGCCCAACCAGGCGCAGATGCGGATCCTGAAAGA
GACGGAGCTGAGGAAGGTGAAGGTGCTTGGATCTGGCGCTTTTGGCACAG
TCTACAAGGGCATCTGGATCCCTGATGGGGAGAATGTGAAAATTCCAGTG
GCCATCAAAGTGTTGAGGGAAAACACATCCCCCAAAGCCAACAAAGAAAT
CTTAGACGAAGCATACGTGATGGCTGGTGTGGGCTCCCCATATGTCTCCC
GCCTTCTGGGCATCTGCCTGACATCCACGGTGCAGCTGGTGACACAGCTT
ATGCCCTATGGCTGCCTCTTAGACCATGTCCGGGAAAACCGCGGACGCCT
GGGCTCCCAGGACCTGCTGAACTGGTGTATGCAGATTGCCAAGGGGATGA
GCTACCTGGAGGATGTGCGGCTCGTACACAGGGACTTGGCCGCTCGGAAC
GTGCTGGTCAAGAGTCCCAACCATGTCAAAATTACAGACTTCGGGCTGGC
TCGGCTGCTGGACATTGACGAGACAGAGTACCATGCAGATGGGGGCAAGG
TGCCCATCAAGTGGATGGCGCTGGAGTCCATTCTCCGCCGGCGGTTCACC
CACCAGAGTGATGTGTGGAGTTATGGTGTGACTGTGTGGGAGCTGATGAC
TTTTGGGGCCAAACCTTACGATGGGATCCCAGCCCGGGAGATCCCTGACC
TGCTGGAAAAGGGGGAGCGGCTGCCCCAGCCCCCCATCTGCACCATTGAT
GTCTACATGATCATGGTCAAATGTTGGATGATTGACTCTGAATGTCGGCC
AAGATTCCGGGAGTTGGTGTCTGAATTCTCCCGCATGGCCAGGGACCCCC
AGCGCTTTGTGGTCATCCAGAATGAGGACTTGGGCCCAGCCAGTCCCTTG
GACAGCACCTTCTACCGCTCACTGCTGGAGGACGATGACATGGGGGACCT
GGTGGATGCTGAGGAGTATCTGGTACCCCAGCAGGQCTTCTTCTGTCCAG
ACCCTGCCCCGGGCGCTGGGGGCATGGTCCACCACAGGCACCGCAGCTCA
TCTACCAGGAGTGGCGGTGGGGACCTGACACTAGGGCTGGAGCCCTCTGA
AGAGGAGGCCCCCAGGTCTCCACTGGCACCCTCCGAAGGGGCTGGCTCCG
ATGTATTTGATGGTGACCTGGGAATGGGGGCAGCCAAGGGGCTGCAAAGC
CTCCCCACACATGACCCCAGCCCTCTACAGCGGTACAGTGAGGACCCCAC
AGTACCCCTGCGCTCTGAGACTGATGGCTACGTTGCCCCCCTGACCTGCA
GCCCCCAGCCTGAATATGTGAACCAGCCAGATGTTCGGCCCCAGCCCCCT
TCGCCCCGAGAGGGCCCTCTGCCTGCTGCCCGACCTGCTGGTGCCACTCT
GGAAAGGGCCAAGACTCTCTCCCCAGGGAAGAATGGGGTCGTCAAAGACG
TTTTTGCCTTTGGGGGTGCCGTGGAGAACCCCGAGTACTTGACACCCCAG
GGAGGAGCTGCCCCTCAGCCCCACCCTCCTCCTGCCTTCAGCCCAGCCTT
CGACAACCTCTATTACTGGGACCAGGACCCACCAGAGCGGGGGGCTCCAC
CCAGCACCTTCAAAGGGACACCTACGGCAGAGAACCCAGAGTACCTGGGT
CTGGACGTGCCAGTG
SEQ ID NO: 3
HUMAN EGFR POLYNUCLEOTIDE SEQUENCE (SEQ ID NO: 4)
CCGGCGCAGCGCGGCCGCAGCAGCCTCCGCCCCCCGCACGGTGTGAGCGC
CCGCCGCGGCCGAGGCGGCCGGAGTCCCGAGCTAGCCCCGGCGGCCGCCG
CCGCCCAGACCGGACGACAGGCCACCTCGTCGGCGTCCGCCCGAGTCCCC
GCCTCGCCGCCAACGCCACAACCACCGCGCACGGCCCCCTGACTCCGTCC
AGTATTGATCGGGAGAGCCGGAGCGAGCTCTTCGGGGAOCAGCGATGCGA
CCCTCCGGGACGGCCGGGGCAGCGCTCCTGGCGCTGCTGGCTGCGCTCTG
CCCGGCGAGTCGGGCTCTGGAGGAAAAGAAAGTTTGCCAAGGCACGAGTA
ACAAGCTCACGCAGTTGGGCACTTTTGAAGATCATTTTCTCAGCCTCCAG
AGGATGTTCAATAACTGTGAGGTGGTCCTTGGGAATTTGGAAATTACCTA
TGTGCAGAGGAATTATGATCTFTCCTTCTTAAAGACCATCCAGGAGGTGG
CTGGTTATGTCCTCATTGCCCTCAACACAGTGGAGCGAATTCCTTTGGAA
AACCTGCAGATCATCAGAGGAAATATGTACTACGAAAATTCCTATGCCTT
AGCAGTCTTATCTAACTATGATGCAAATAAAACCGGACTGAAGGAGCTGC
CCATGAGAAATTTACAGGAAATCCTGCATGGCGCCGTGCGGTTCAGCAAC
AACCCTGCCCTGTGCAATGTGGAGAGCATCCAGTGGCGGGACATAGTCAG
CAGTGACTTTCTCAGCAACATGTCGATGGACTTCCAGAACCACCTGGGCA
GCTGCCAAAAGTGTGATCCAAGCTGTCCCAATGGGAGCTGCTGGGGTGCA
GGAGAGGAGAACTGCCAGAAACTGACCAAAATCATCTGTGCCCAGCAGTG
CTCCGGGCGCTGCCGTGGCAAGTCCCCCAGTGACTGCTGCCACAACCAGT
GTGCTGCAGGCTGCACAGGCCCCCGGGAGAGCGACTGCCTGGTCTGCCGC
AAATTCCGAGACGAAGCCACGTGCAAGGACACCTGCCCCCCACTCATGCT
CTACAACCCCACCACGTACCAGATGGATGTGAACCCCGAGGGCAAATACA
GCTTTGGTGCCACCTGCGTGAAGAAGTGTCCCCGTAATTATGTGGTGACA
GATCACGGCTCGTGCGTCCGAGCCTGTGGGGCCGACAGCTATGAGATGGA
GGAAGACGGCGTCCGCAAGTGTAAGAAGTGCGAAGGGCCTTGCCGCAAAG
TGTGTAACGGAATAGGTATTGGTGAATTTAAAGACTCACTCTCCATAAAT
GCTACGAATATTAAACACTTCAAAAACTGCACCTCCATCAGTGGCGATCT
CCACATCCTGCCGGTGGCATTTAGGGGTGACTCCTTCACACATACTCCTC
CTCTGGATCCACAGGAACTGGATATTCTGAAAACCGTAAAGGAAATCACA
GGTTTGAGCTGAATTATCACATGAATATAAATGGGAAATCAGTGTTTTAG
AGAGAGAACTTTTCGACATATTTCCTGTTCCCTTGGAATAAAAACATTTC
TTCTGAAATTTTACCGTTAA
HUMAN VEGF POLYNUCLEOTIDE SEQUENCE (SEQ ID NO: 5)
AAGAGCTCCAGAGAGAAGTCGAGGAAGAGAGAGACGGGGTCAGAGAGAGC
GCGCGGGCGTGCGAGCAGCGAAAGCGACAGGGGCAAAGTGAGTGACCTGC
TTTTGGGGGTGACCGCCGGAGCGCGGCGTGAGCCCTCCCCCTTGGGATCC
CGCAGCTGACCAGTCGCGCTGACGGACAGACAGACAGACACCGCCCCCAG
CCCCAGTTACCACCTCCTCCCCGGCCGGCGGCGGACAGTGGACGCGGCGG
CGAGCCGCGGGCAGGGGCCGGAGCCCGCCCCCGGAGGCGGGGTGGAGGGG
GTCGGAGCTCGCGGCGTCGCACTGAAACTTTTCGTCCAACTTCTGGGCTG
TTCTCGCTTCGGAGGAGCCGTGGTCCGCGCGGGGGAAGCCGAGCCGAGCG
GAGCCGCGAGAAGTGCTAGCTCGGGCCGGGAGGAGCCGCAGCCGGAGGAG
GGGGAGGAGGAAGAAGAGAAGGAAGAGGAGAGGGGGCCGCAGTGGCGACT
CGGCGCTCGGAAGCCGGGCTCATGGACGGGTGAGGCGGCGGTGTGCGCAG
ACAGTGCTCCAGCGCGCGCGCTCCCCAGCCCTGGCCCGGCCTCGGGCCGG
GAGGAAGAGTAGCTCGCCGAGGCGCCGAGGAGAGCGGGCCGCCCCACAGC
CCGAGCCGGAGAGGGACGCGAGCCGCGCGCCCCGGTCGGGCCTCCGAAAC
CATGAACTTTCTGCTGTCTTGGGTGCATTGGAGCCTTGCCTTGCTGCTCT
ACCTCCACCATGCCAAGTGGTCCCAGGCTGCACCCATGGCAGAAGGAGGA
GGGCAGAATCATCACGAAGTGGTGAAGTTCATGGATGTCTATCAGCGCAG
CTACTGCCATCCAATCGAGACCCTGGTGGACATCTTCCAGGAGTACCCTG
ATGAGATCGAGTACATCTTCAAGCCATCCTGTGTGCCCCTGATGCGATGC
GGGGGCTGCTCCAATGACGAGGGCCTGGAGTGTGTGCCCACTGAGGAGTC
CAACATCACCATGCAGATTATGCGGATCAAACCTCACCAAGGCCAGCACA
TAGGAGAGATGAGCTTCCTACAGCACAACAAATGTGAATGCAGACCAAAG
AAAGATAGAGCAAGACAAGAAAATCCCTGTGGGCCTTGCTCAGAGCGGAG
AAAGCATTTGTTTGTACAAGATCCGCAGACGTGTAAATGTTCCTGCAAAA
ACACACACTCGCGTTGCAAGGCGAGGCAGCTTGAGTTAAACGAACGTACT
TGCAGATGTGACAAGCCGAGGCGGTGAGCCGGGCAGGAGGAAGGAGCCTC
CCTCAGGGTTTCGGGAACCAGATCTCTCTCCAGGAAAGACTGATACAGAA
CGATCGATACAGAAACCACGCTGCCGCCACCACACCATCACCATCGACAG
AACAGTCCTTAATCCAGAAACCTGAAATGAAGGAAGAGGAGACTCTGCGC
AGAGCACTTTGGGTCCGGAGGGCGAGACTCCGGCGGAAGCATTCCCGGGC
GGGTGACCCAGCACGGTCCCTCTTGGAATTGGATTCGCCATTTTATTTTT
CTTGCTGCTAAATCACCGAGCCCGGAAGATTAGAGAGTTTTATTTCTGGG
ATTCCTGTAGACACACCCACCCACATACATACATTTATATATATATATAT
TATATATATATAAAAATAAATATCTCTATTTTATATATATAAAATATATA
TATTCTTTTTTTAAATTAACAGTGCTAATGTTATTGGTGTCTTCACTGGA
TGTATTTGACTGCTGTGGACTTGAGTTGGGAGGGGAATGTTCCCACTCAG
ATCCTGACAGGGAAGAGGAGGAGATGAGAGACTCTGGCATGATCTTTTTT
TTGTCCCACTTGGTGGGGCCAGGGTCCTCTCCCCTGCCCAAGAATGTGCA
AGGCCAGGGCATGGGGGCAAATATGACCCAGTTTTGGGAACACCGACAAA
CCCAGCCCTGGCGCTGAGCCTCTCTACCCCAGGTCAGACGGACAGAAAGA
CAAATCACAGGTTCCGGGATGAGGACACCGGCTCTGACCAGGAGTTTGGG
GAGCTTCAGGACATTGCTGTGCTTTGGGGATTCCCTCCACATGCTGCACG
CGCATCTCGCCCCCAGGGGCACTGCCTGGAAGATTCAGGAGCCTGGGCGG
CCTTCGCTTACTCTCACCTGCTTCTGAGTTGCCCAGGAGGCCACTGGCAG
ATGTCCCGGCGAAGAGAAGAGACACATTGTTGGAAGAAGCAGCCCATGAC
AGCGCCCCTTCCTGGGACTCGCCCTCATCCTCTTCCTGCTCCCCTTCCTG
GGGTGCAGCCTAAAAGGACCTATGTCCTCACACCATTGAAACCACTAGTT
CTGTCCCCCCAGGAAACCTGGTTGTGTGTGTGTGAGTGGTTGACCTTCCT
CCATCCCCTGGTCCTTCCCTTCCCTTCCCGAGGCACAGAGAGACAGGGCA
GGATCCACGTGCCCATTGTGGAGGCAGAGAAAAGAGAAAGTGTTTTATAT
ACGGTACTTATTTAATATCCCTTTTTAATTAGAAATTAGAACAGTTAATT
TAATTAAAGAGTAGGGTTTTTTTTCAGTATTCTTGGTTAATATTTAATTT
CAACTATTTATGAGATGTATCTTTTGCTCTCTCTTGCTCTCTTATTTGTA
CCGGTTTTTGTATATAAAATTCATGTTTCCAATCTCTCTCTCCCTGATCG
GTGACAGTCACTAGCTTATCTTGAACAGATATTTAATTTTGCTAACACTC
AGCTCTGCCCTCCCCGATCCCCTGGCTCCCCAGCACACATTCCTTTGAAA
GAGGGTTTCAATATACATCTACATACTATATATATATTGGGCAACTTGTA
TTTGTGTGTATATATATATATATATGTTTATGTATATATGTGATCCTGAA
AAAATAAACATCGCTATTCTGTTTTTTATATGTTCAAACCAAACAAGAAA
AAATAGAGAATTCTACATACTAAATCTCTCTCCTTTTTTAATTTTAATAT
TTGTTATCATTTATTTATTGGTGCTACTGTTTATCCGTAATAATTGTGGG
GAAAAGATATTAACATCACGTCTTTGTCTCTAGTGCAGTTTTTCGAGATA
TTCCGTAGTACATATTTATTTTTAAACAACGACAAAGAAATACAGATATA
TCTTA
HUMAN FLT FMS-LIKE TYROSINE KINASE-3 (FLT3)
POLYNUCLEOTIDE SEQUENCE (SEQ ID NO: 6)
CGAGGCGGCATCCGAGGGCTGGGCCGGCGCCCTGGGGGACCCCGGGCTCC
GGAGGCCATGCCGGCGTTGGCGCGCGACGCGGGCACCGTGCCGCTGCTCG
TTGTTTTTTCTGCAATGATATTTGGGACTATTACAAATCAAGATCTGCCT
GTGATCAAGTGTGTTTTAATCAATCATAAGAACAATGATTCATCAGTGGG
GAAGTCATCATCATATCCCATGGTATCAGAATCCCCGGAAGACCTCGGGT
GTGCGTTGAGACCCCAGAGCTCAGGGACAGTGTACGAAGCTGCCGCTGTG
GAAGTGGATGTATCTGCTTCCATCACACTGCAAGTGCTGGTCGATGCCCC
AGGGAACATTTCCTGTCTCTGGGTCTTTAAGCACAGCTCCCTGAATTGCC
AGCCACATTTTGATTTACAAAACAGAGGAGTTGTTTCCATGGTCATTTTG
AAAATGACAGAAACCCAAGCTGGAGAATACCTACTTTTTATTCAGAGTGA
AGCTACCAATTACACAATATTGTTTACAGTGAGTATAAGAAATACCCTGC
TTTACACATTAAGAAGACCTTACTTTAGAAAAATGGAAAACCAGGACGCC
CTGGTCTGCATATCTGAGAGCGTTCCAGAGCCGATCGTGGAATGGGTGCT
TTGCGATTCACAGGGGGAAAGCTGTAAAGAAGAAAGTCCAGCTGTTGTTA
AAAAGGAGGAAAAAGTGCTTCATGAATTATTTGGGACGGACATAAGGTGC
TGTGCCAGAAATGAACTGGGCAGGGAATGCACCAGGCTGTTCACAATAGA
TCTAAATCAAACTCCTCAGACCACATTGCCACAATTATTTCTTAAAGTAG
GGGAACCCTTATGGATAAGGTGCAAAGCTGTTCATGTGAACCATGGATTC
GGGCTCACCTGGGAATTAGAAAACAAAGCACTCGAGGAGGGCAACTACTT
TGAGATGAGTACCTATTCAACAAACAGAACTATGATACGGATTCTGTTTG
CTTTTGTATCATCAGTGGCAAGAAACGACACCGGATACTACACTTGTTCC
TCTTCAAAGCATCCCAGTCAATCAGCTTTGGTTACCATCGTAGGAAAGGG
ATTTATAAATGCTACCAATTCAAGTGAAGATTATGAAATTGACCAATATG
AAGAGTTTTGTTTTTCTGTCAGGTTTAAAGCCTACCCACAAATCAGATGT
ACGTGGACCTTCTCTCGAAAATCATTTCCTTGTGAGCAAAAGGGTCTTGA
TAACGGATACAGCATATCCAAGTTTTGCAATCATAAGCACCAGCCAGGAG
AATATATATTCCATGCAGAAAATGATGATGCCCAATTTACCAAAATGTTC
ACGCTGAATATAAGAAGGAAACCTCAAGTGCTCGCAGAAGCATCGGCAAG
TCAGGCGTCCTGTTTCTCGGATGGATACCCATTACCATCTTGGACCTGGA
AGAAGTGTTCAGACAAGTCTCCCAACTGCACAGAAGAGATCACAGAAGGA
GTCTGGAATAGAAAGGCTAACAGAAAAGTGTTTGGACAGTGGGTGTCGAG
CAGTACTCTAAACATGAGTGAAGCCATAAAAGGGTTCCTGGTCAAGTGCT
GTGCATACAATTCCCTTGGCACATCTTGTGAGACGATCCTTTTAAACTCT
CCAGGCCCCTTCCCTTTCATCCAAGACAACATCTCATTCTATGCAACAAT
TGGTGTTTGTCTCCTCTTCATTGTCGTTTTAACCCTGCTAATTTGTCACA
AGTACAAAAAGCAATTTAGGTATGAAAGCCAGCTACAGATGGTACAGGTG
ACCGGCTCCTCAGATAATGAGTACTTCTACGTTGATTTCAGAGAATATGA
ATATGATCTCAAATGGGAGTTTCCAAGAGAAAATTTAGAGTTTGGGAAGG
TACTAGGATCAGGTGCTTTTGGAAAAGTGATGAACGCAACAGCTTATGGA
ATTAGCAAAACAGGAGTCTCAATCCAGGTTGCCGTCAAAATGCTGAAAGA
AAAAGCAGACAGCTCTGAAAGAGAGGCACTCATGTCAGAACTCAAGATGA
TGACCCAGCTGGGAAGCCACGAGAATATTGTGAACCTGCTGGGGGCGTGC
ACACTGTCAGGACCAATTTACTTGATTTTTGAATACTGTTGCTATGGTGA
TCTTCTCAACTATCTAAGAAGTAAAAGAGAAAAATTTCACAGGACTTGGA
CAGAGATTTTCAAGGAACACAATTTCAGTTTTTACCCCACTTTCCAATCA
CATCCAAATTCCAGCATGCCTGGTTCAAGAGAAGTTCAGATACACCCGGA
CTCGGATCAAATCTCAGGGCTTCATGGGAATTCATTTCACTCTGAAGATG
AAATTGAATATGAAAACCAAAAAAGGCTGGAAGAAGAGGAGGACTTGAAT
GTGCTTACATTTGAAGATCTTCTTTGCTTTGCATATCAAGTTGCCAAAGG
AATGGAATTTCTGGAATTTAAGTCGTGTGTTCACAGAGACCTGGCCGCCA
GGAACGTGCTTGTCACCCACGGGAAAGTGGTGAAGATATGTGACTTTGGA
TTGGCTCGAGATATCATGAGTGATTCCAACTATGTTGTCAGGGGCAATGC
CCGTCTGCCTGTAAAATGGATGGCCCCCGAAAGCCTGTTTGAAGGCATCT
ACACCATTAAGAGTGATGTCTGGTCATATGGAATATTACTGTGGGAAATC
TTCTCACTTGGTGTGAATCCTTACCCTGGCATTCCGGTTGATGCTAACTT
CTACAAACTGATTCAAAATGGATTTAAAATGGATCAGCCATTTTATGCTA
CAGAAGAAATATACATTATAATGCAATCCTGCTGGGCTTTTGACTCAAGG
AAACGGCCATCCTTCCCTAATTTGACTTCGTTTTTAGGATGTCAGCTGGC
AGATGCAGAAGAAGCGATGTATCAGAATGTGGATGGCCGTGTTTCGGAAT
GTCCTCACACCTACCAAAACAGGCGACCTTTCAGCAGAGAGATGGATTTG
GGGCTACTCTCTCCGCAGGCTCAGGTCGAAGATTCGTAGAGGAACAATTT
AGTTTTAAGGACTTCATCCCTCCACCTATCCCTAACAGGCTGTAGATTAC
CAAAACAAGATTAATTTCATCACTAAAAGAAAATCTATTATCAACTGCTG
CTTCACCAGACTTTTCTCTAGAAGCCGTCTGCGTTTACTCTTGTTTTCAA
AGGGACTTTTGTAAAATCAAATCATCCTGTCACAAGGCAGGAGGAGCTGA
TAATGAACTTTATTGGAGCATTGATCTGCATCCAAGGCCTTCTCAGGCCG
GCTTGAGTGAATTGTGTACCTGAAGTACAGTATATTCTTGTAAATACATA
AAACAAAAGCATTTTGCTAAGGAGAAGCTAATATGATTTTTTAAGTCTAT
GTTTTAAAATAATATGTAAATTTTTCAGCTATTTAGTGATATATTTTATG
GGTGGGAATAAAATTTCTACTACAG
HUMAN MYC POLYNUCLEOTIDE SEQUENCE (SEQ ID NO: 7)
AAGTGCTGGGATTACAGGTGTGAGCCAGGGCACCAGGCTTAGATGTGGCT
CTTTGGGGAGATAATTTTGTCCAGAGACCTTTCTAACGTATTCATGCCTT
GTATTTGTACAGCATTAATCTGGTAATTGATTATTTTAATGTAACCTTGC
TAAAGGAGTGATTTCTATTTCCTTTCTTAAAGAGGAGGAACAAGAAGATG
AGGAAGAAATCGATGTTGTTTCTGTGGAAAAGAGGCAGGCTCCTGGCAAA
AGGTCAGAGTCTGGATCACCTTCTGCTGGAGGCCACAGCAAACCTCCTCA
CAGCCCACTGGTCCTCAAGAGGTGCCACGTCTCCACACATCAGCACAACT
ACGCAGCGCCTCCCTCCACTCGGAAGGACTATCCTGCTGCCAAGAGGGTC
AAGTTGGACAGTGTCAGAGTCCTGAGACAGATCAGCAACAACCGAAAATG
CACCAGCCCCAGGTCCTCGGACACCGAGGAGAATGTCAAGAGGCGAACAC
ACAACGTCTTGGAGCGCCAGAGGAGGAACGAGCTAAAACGGAGCTTTTTT
GCCCTGCGTGACCAGATCCCGGAGTTGGAAAACAATGAAAAGGCCCCCAA
GGTAGTTATCCTTAAAAAAGCCACAGCATACATCCTGTCCGTCCAAGCAG
AGGAGCAAAAGCTCATTTCTGAAGAGGACTTGTTGCGGAAACGACGAGAA
CAGTTGAAACACAAACTTGAACAGCTACGGAACTCTTGTGCGTAAGGAAA
AGTAAGGAAAACGATTCCTTCTAACAGAAATGTCCTGAGCAATCACCTAT
GAACTTGTTTCAAATGCATGATCAAATGCAACCTCACAACCTTGGCTGAG
TCTTGAGACTGAAAGATTTAGCCATAATGTAAACTGCCTCAAATTGGACT
TTGGGCATAAAAGAACTTTTTTATGCTTACCATCTTTTTTTTTTCTTTAA
CAGATTTGTATTTAAGAATTGTTTTTAAAAAATTTTAAGATTTACACAAT
GTTTCTCTGTAAATATTGCCATTAAATGTAAATAACTTTAATAAAACGTT
TATAGCAGTTACACAGAATTTCAATCCTAGTATATAGTACCTAGTATTAT
AGGTACTATAAACCCTAATTTTTTTTATTTAAGTACATTTTGCTTTTTAA
AGTTGATTTTTTTCTATTGTTTTTAGAAAAAATAAAATAACTGGCAAATA
TATCATTGAGCCAAATCTTAAGTTGTGAATGTTTTGTTTCGTTTCTTCCC
CCTCCCAACCACCACCATCCCTGTTTGTTTTCATCAATTGCCCCTTCAGA
GGGTGGTCTTAAGAAAGGCAAGAGTTTTCCTCTGTTGAAATGGGTCTGGG
GGCCTTAAGGTCTTTAAGTTCTTGGAGGTTCTAAGATGCTTCCTGGAGAC
TATGATAACAGCCGAAGTTGACAGTTAGAAGGAATGGCAGAAGGCAGGTG
AGAAGGTGAGAGGTAGGCAAAGGAGATACAAGAGGTCAAAGGTAGCAGTT
AAGTACACAAAGAGGCATAAGGACTGGGGAGTTGGGAGGAAGGTGAGGAA
GAAACTCCTGTTACTTTAGTTAACCAGTGCCAGTCCCCTGCTCACTCCAA
A
HUMAN UROKINASE PLASMINOGEN ACTIVATOR (UPA)
POLYNUCLEOTIDE SEQUENCE (SEQ ID NO: 8)
CCCGGGCCAGGGTCCACCTGTCCCCGCAGCGCCGGCTCGCGCCCTCCTGC
CGCAGCCACCGAGCCGCCGTCTAGCGCCCCGACCTCGCCACCATGAGAGC
CCTGCTGGCGCGCCTGCTTCTCTGCGTCCTGGTCGTGAGCGACTCCAAAG
GCAGCAATGAACTTCATCAAGTTCCATCGAACTGTGACTGTCTAAATGGA
GGAACATGTGTGTCCAACAAGTACTTCTCCAACATTCACTGGTGCAACTG
CCCAAAGAAATTCGGAGGGCAGCACTGTGAAATAGATAAGTCAAAAACCT
GCTATGAGGGGAATGGTCACTTTTACCGAGGAAAGGCCAGCACTGACACC
ATGGGCCGGCCCTGCCTGCCCTGGAACTCTGCCACTGTCCTTCAGCAAAC
GTACCATGCCCACAGATCTGATGCTCTTCAGCTGGGCCTGGGGAAACATA
ATTACTGCAGGAACCCAGACAACCGGAGGCGACCCTGGTGCTATGTGCAG
GTGGGCCTAAAGCCGCTTGTCCAAGAGTGCATGGTGCATGACTGCGCAGA
TGGAAAAAAGCCCTCCTCTCCTCCAGAAGAATTAAAATTTCAGTGTGGCC
AAAAGACTCTGAGGCCCCGCTTTAAGATTATTGGGGGAGAATTCACCACC
ATCGAGAACCAGCCCTGGTTTGCGGCCATCTACAGGAGGCACCGGGGGGG
CTCTGTCACCTACGTGTGTGGAGGCAGCCTCATCAGCCCTTGCTGGGTGA
TCAGCGCCACACACTGCTTCATTGATTACCCAAAGAAGGAGGACTACATC
GTCTACCTGGGTCGCTCAAGGCTTAACTCCAACACGCAAGGGGAGATGAA
GTTTGAGGTGGAAAACCTCATCCTACACAAGGACTACAGCGCTGACACGC
TTGCTCACCACAACGACATTGCCTTGCTGAAGATCCGTTCCAAGGAGGGC
AGGTGTGCGCAGCCATCCCGGACTATACAGACCATCTGCCTGCCCTCGAT
GTATAACGATCCCCAGTTTGGCACAAGCTGTGAGATCACTGGCTTTGGAA
AAGAGAATTCTACCGACTATCTCTATCCGGAGCAGCTGAAAATGACTGTT
GTGAAGCTGATTTCCCACCGGGAGTGTCAGCAGCCCCACTACTACGGCTC
TGAAGTCACCACCAAAATGCTGTGTGCTGCTGACCCACAGTGGAAAACAG
ATTCCTGCCAGGGAGACTCAGGGGGACCCCTCGTCTGTTCCCTCCAAGGC
CGCATGACTTTGACTGGAATTGTGAGCTGGGGCCGTGGATGTGCCCTGAA
GGACAAGCCAGGCGTCTACACGAGAGTCTCACACTTCTTACCCTGGATCC
GCAGTCACACCAAGGAAGAGAATGGCCTGGCCCTCTGAGGGTCCCCAGGG
AGGAAACGGGCACCACCCGCTTTCTTGCTGGTTGTCATTTTTGCAGTAGA
GTCATCTCCATCAGCTGTAAGAAGAGACTGGGAAGATAGGCTCTGCACAG
ATGGATTTGCCTGTGCCACCCACCAGGGTGAACGACAATAGCTTTACCCT
CAGGCATAGGCCTGGGTGCTGGCTGCCCAGACCCCTCTGGCCAGGATGGA
GGGGTGGTCCTGACTCAACATGTTACTGACCAGCAACTTGTCTTTTTCTG
GACTGAAGCCTGCAGGAGTTAAAAAGGGCAGGGCATCTCCTGTGCATGGG
TGAAGGGAGAOCCAGCTCCCCCGACGGTGGGCATTTGTGAGGCCCATGGT
TGAGAAATGAATAATTTCCCAATTAGGAAGTGTAACAGCTGAGGTCTCTT
GAGGGAGCTTAGCCAATGTGGGAGCAGCGGTTTGGGGAGCAGAGACACTA
ACGACTTCAGGGCAGGGCTCTGATATTCCATGAATGTATCAGGAAATATA
TATGTGTGTGTATGTTTGCACACTTGTGTGTGGGCTGTGAGTGTAAGTGT
GAGTAAGAGCTGGTGTCTGATTGTTAAGTCTAAATATTTCCTTAAACTGT
GTGGACTGTGATGCCACACAGAGTGGTCTTTCTGGAGAGGTTATAGGTCA
CTCCTGGGGCCTCTTGGGTCCCCCACGTGACAGTGCCTGGGAATGTATTA
TTCTGCAGCATGACCTGTGACCAGCACTGTCTCAGTTTCACTTTCACATA
GATGTCCCTTTCTTGGCCAGTTATCCCTTCCTTTTAGCCTAGTTCATCCA
ATCCTCACTGGGTGGGGTGAGGACCACTCCTTACACTGAATATTTATATT
TCACTATTTTTATTTATATTTTTGTAATTTTAAATAAAAGTGATCAATAA
AATGTGATTTTTCTGATGAA
HUMAN PLASMINOGEN ACTIVATOR INHIBITOR (PAI-1)
POLYNUCLEOTIDE SEQUENCE SEQ ID NO: 9)
GAATTCCTGCAGCTCAGCAGCCGCCGCCAGAGCAGGACGAACCGCCAATC
GCAAGGCACCTCTGAGAACTTCAGGATGCAGATGTCTCCAGCCCTCACCT
GCCTAGTCCTGGGCCTGGCCCTTGTCTTTGGTGAAGGGTCTGCTGTGCAC
CATCCCCCATCCTACGTGGCCCACCTGGCCTCAGACTTCGGGGTGAGGGT
GTTTCAGCAGGTGGCGCAGGCCTCCAAGGACCGCAACGTGGTTTTCTCAC
CCTATGGGGTGGCCTCGGTGTTGGCCATGCTCCAGCTGACAACAGGAGGA
GAAACCCAGCAGCAGATTCAAGCAGCTATGGGATTCAAGATTGATGACAA
GGGCATGGCCCCCGCCCTCCGGCATCTGTACAAGGAGCTCATGGGGCCAT
GGAACAAGGATGAGATCAGCACCACAGACGCGATCTTCGTCCAGCGGGAT
CTGAAGCTGGTCCAGGGCTTCATGCCCCACTTCTTCAGGCTGTTCCGGAG
CACGGTCAAGCAAGTGGACTTTTCAGAGGTGGAGAGAGCCAGATTCATCA
TCAATGACTGGGTGAAGACACACACAAAAGGTATGATCAGCAACTTGCTT
GGGAAAGGAGCCGTGGACCAGCTGACACGGCTGGTGCTGGTGAATGCCCT
CTACTTCAACGGCCAGTGGAAGACTCCCTTCCCCGACTCCAGCACCCACC
GCCGCCTCTTCCACAAATCAGACGGCAGCACTGTCTCTGTGCCCATGATG
GCTCAGACCAACAAGTTCAACTATACTGAGTTCACCACGCCCGATGGCCA
TTACTACGACATCCTGGAACTGCCCTACCACGGGGACACCCTCAGCATGT
TCATTGCTGCCCCTTATGAAAAAGAGGTGCCTCTCTCTGCCCTCACCAAC
ATTCTGAGTGCCCAGCTCATCAGCCACTGGAAAGGCAACATGACCAGGCT
GCCCCGCCTCCTGGTTCTGCCCAAGTTCTCCCTGGAGACTGAAGTCGACC
TCAGGAAGCCCCTAGAGAACCTGGGAATGACCGACATGTTCAGACAGTTT
CAGGCTGACTTCACGAGTCTTTCAGACCAAGAGCCTCTCCACGTCGCGCA
GGCGCTGCAGAAAGTGAAGATCGAGGTGAACGAGAGTGGCACGGTGGCCT
CCTCATCCACAGCTGTCATAGTCTCAGCCCGCATGGCCCCCGAGGAGATC
ATCATGGACAGACCCTTCCTCTTTGTGGTCCGGCACAACCCCACAGGAAC
AGTCCTTTTCATGGGCCAAGTGATGGAACCCTGACCCTGGGGAAAGACGC
CTTCATCTGGGACAAAACTGGAGATGCATCGGGAAAGAAGAAACTCCGAA
GAAAAGAATTTTAGTGTTAATGACTCTTTCTGAAGGAAGAGAAGACATTT
GCCTTTTGTTAAAAGATGGTAAACCAGATCTGTCTCCAAGACCTTGGCCT
CTCCTTGGAGGACCTTTAGGTCAAACTCCCTAGTCTCCACCTGAGACCCT
GGGAGAGAAGTTTGAAGCACAACTCCCTTAAGGTCTCCAAACCAGACGGT
GACGCCTGCGGGACCATCTGGGGCACCTGCTTCCACCCGTCTCTCTGCCC
ACTCGGGTCTGCAGACCTGGTTCCCACTGAGGCCCTTTGCAGGATGGAAC
TACGGGGCTTACAGGAGCTTTTGTGTGCCTGGTAGAAACTATTTCTGTTC
CAGTCACATTGCCATCACTCTTGTACTGCCTGCCACCGCGGAGGAGGCTG
GTGACAGGCCAAAGGCCAGTGGAAGAAACACCCTTTCATCTCAGAGTCCA
CTGTGGCACTGGCCACCCCTCCCCAGTACAGGGGTGCTGCAGGTGGCAGA
GTGAATGTCCCCCATCATGTGGCCCAACTCTCCTGGCCTGGCCATCTCCC
TCCCCAGAAACAGTGTGCATGGGTTATTTTGGAGTGTAGGTGACTTGTTT
ACTCATTGAAGCAGATTTCTGCTTCCTTTTATTTTTATAGGAATAGAGGA
AGAAATGTCAGATGCGTGCCCAGCTCTTCACCCCCCAATCTCTTGGTGGG
GAGGGGTGTACCTAAATATTTATCATATCCTTGCCCTTGAGTGCTTGTTA
GAGAGAAAGAGAACTACTAAGGAAAATAATATTATTTAAACTCGCTCCTA
GTGTTTCTTTGTGGTCTGTGTCACCGTATCTCAGGAAGTCCAGCCACTTG
ACTGGCACACACCCCTCCGGACATCCAGCGTGACGGAGCCCACACTGCCA
CCTTGTGGCCGCCTGAGACCCTCGCGCCCCCCGCGCCCCCCGCGCCCCTC
TTTTTCCCCTTGATGGAAATTGACCATACAATTTCATCCTCCTTCAGGGG
ATCAAAAGGACGGAGTGGGGGGACAGAGACTCAGATGAGGACAGAGTGGT
TTCCAATGTGTTCAATAGATTTAGGAGCAGAAATGCAAGGGGCTGCATGA
CCTACCAGGACAGAACTTTCCCCAATTACAGGGTGACTCACAGCCGCATT
GGTGACTCACTTCAATGTGTCATTTCCGGCTGCTGTGTGTGAGCAGTGGA
CACGTGAGGGGGGGGTGGGTGAGAGAGACAGGCAGCTCGGATTCAACTAC
CTTAGATAATATTTCTGAAAACCTACCAGCCAGAGGGTAGGGCACAAAGA
TGGATGTAATGCACTTTGGGAGGCCAAGGCGGGAGGATTGCTTGAGCCCA
GGAGTTCAAGACCAGCCTGGGCAACATACCAAGACCCCCGTCTCTTTAAA
AATATATATATTTTAAATATACTTAAATATATATTTCTAATATCTTTAAA
TATATATATATATTTTAAAGACCAATTTATGGGAGAATTGCACACAGATG
TGAAATGAATGTAATCTAATAGAAGC
HUMAN BRCA1 POLYNUCLEOTIDE SEQUENCE
(SEQ ID NO: 10)
AAAACTGCGACTGCGCGGCGTGAGCTCGCTGAGACTTCCTGGACCCCGCA
CCAGGCTGTGGGGTTTCTCAGATAACTGGGCCCCTGCGCTCAGGAGGCCT
TCACCCTCTGCTCTGGGTAAAGTTCATTGGAACAGAAAGAAATGGATTTA
TCTGCTCTTCGCGTTOAAGAAGTACAAAATGTCATTAATGCTATGCAGAA
AATCTTAGAGTGTCCCATCTGTCTGGAGTTGATCAAGGAACCTGTCTCCA
CAAAGTGTGACCACATATTTTGCAAATTTTGCATGCTGAAACTTCTCAAC
CAGAAGAAAGGGCCTTCACAGTGTCCTTTATGTAAGAATGATATAACCAA
AAGGAGCCTACAAGAAAGTACGAGATTTAGTCAACTTGTTGAAGAGCTAT
TGAAAATCATTTGTGCTTTTCAGCTTGACACAGGTTTGGAGTATGCAAAC
AGCTATAATTTTGCAAAAAAGGAAAATAACTCTCCTGAACATCTAAAAGA
TGAAGTTTCTATCATCCAAAGTATGGGCTACAGAAACCGTGCCAAAAGAC
TTCTACAGAGTGAACCCGAAAATCCTTCCTTGCAGGAAACCAGTCTCAGT
GTCCAACTCTCTAACCTTGGAACTGTGAGAACTCTGAGGACAAAGCAGCG
GATACAACCTCAAAAGACGTCTGTCTACATTGAATTGGGATCTGATTCTT
CTGAAGATACCGTTAATAAGGCAACTTATTGCAGTGTGGGAGATCAAGAA
TTGTTACAAATCACCCCTCAAGGAACCAGGGATGAAATCAGTTTGGATTC
TGCAAAAAAGGCTGCTTGTGAATTTTCTGAGACGGATGTAACAAATACTG
AACATCATCAACCCAGTAATAATGATTTGAACACCACTGAGAAGCGTGCA
GCTGAGAGGCATCCAGAAAAGTATCAGGGTAGTTCTGTTTCAAACTTGCA
TGTGGAGCCATGTGGCACAAATACTCATGCCAGCTCATTACAGCATGAGA
ACAGCAGTTTATTACTCACTAAAGACAGAATGAATGTAGAAAAGGCTGAA
TTCTGTAATAAAAGCAAACAGCCTGGCTTAGCAAGGAGCCAACATAACAG
ATGGGCTGGAAGTAAGGAAACATGTAATGATAGGCGGACTCCCAGCACAG
AAAAAAAGGTAGATCTGAATGCTGATCCCCTGTGTGAGAGAAAAGAATGG
AATAAGCAGAAACTGCCATGCTCAGAGAATCCTAGAGATACTGAAGATGT
TCCTTGGATAACACTAAATAGCAGCATTCAGAAAGTTAATGAGTGGTTTT
CCAGAAGTGATGAACTGTTAGGTTCTGATGACTCACATGATGGGGAGTCT
GAATCAAATGCCAAAGTAGCTGATGTATTGGACGTTCTAAATGAGGTAGA
TGAATATTCTGGTTCTTCAGAGAAAATAGACTTACTGGCCAGTGATCCTC
ATGAGGCTTTAATATGTAAAAGTGAAAGAGTTCACTCCAAATCAGTAGAG
AGTAATATTGAAGACAAAATATTTGGGAAAACCTATCGGAAGAAGGCAAG
CCTCCCCAACTTAAGCCATGTAACTGAAAATCTAATTATAGGAGCATTTG
TTACTGAGCCACAGATAATACAAGAGCGTCCCCTCACAAATAAATTAAAG
CGTAAAAGGAGACCTACATCAGGCCTTCATCCTGAGGATTTTATCAAGAA
AGCAGATTTGGCAGTTCAAAAGACTCCTGAAATGATAAATCAGGGAACTA
ACCAAACGGAGCAGAATGGTCAAGTGATGAATATTACTAATAGTGGTCAT
GAGAATAAAACAAAAGGTGATTCTATTCAGAATGAGAAAAATCCTAACCC
AATAGAATCACTCGAAAAAGAATCTGCTTTCAAAACGAAAGCTGAACCTA
TAAGCAGCAGTATAAGCAATATGGAACTCGAATTAAATATCCACAATTCA
AAAGCACCTAAAAAGAATAGGCTGAGGAGGAAGTCTTCTACCAGGCATAT
TCATGCGCTTGAACTAGTAGTCAGTAGAAATCTAAGCCCACCTAATTGTA
CTGAATTGCAAATTGATAGTTGTTCTAGCAGTGAAGAGATAAAGAAAAAA
AAGTACAACCAAATGCCAGTCAGGCACAGCAGAAACCTACAACTCATGGA
AGGTAAAGAACCTGCAACTGGAGCCAAGAAGAGTAACAAGCCAAATGAAC
AGACAAGTAAAAGACATGACAGCGATACTTTCCCAGAGCTGAAGTTAACA
AATGCACCTGGTTCTTTTACTAAGTGTTCAAATACCAGTGAACTTAAAGA
ATTTGTCAATCCTAGCCTTCCAAGAGAAGAAAAAGAAGAGAAACTAGAAA
CAGTTAAAGTGTCTAATAATGCTGAAGACCCCAAAGATCTCATGTTAAGT
GGAGAAAGGGTTTTGCAAACTGAAAGATCTGTAGAGAGTAGCAGTATTTC
ATTGGTACCTGGTACTGATTATGGCACTCAGGAAAGTATCTCGTTACTGG
AAGTTAGCACTCTAGGGAAGGCAAAAACAGAACCAAATAAATGTGTGAGT
CAGTGTGCAGCATTTGAAAACCCCAAGGGACTAATTCATGGTTGTTCCAA
AGATAATAGAAATGACACAGAAGGCTTTAAGTATCCATTGGGACATGAAG
TTAACCACAGTCGGGAAACAAGCATAGAAATGGAAGAAAGTGAACTTGAT
GCTCAGTATTTGCAGAATACATTCAAGGTTTCAAAGCGCCAGTCATTTGC
TCCGTTTTCAAATCCAGGAAATGCAGAAGAGGAATGTGCAACATTCTCTG
CCCACTCTGGGTCCTTAAAGAAACAAAGTCCAAAAGTCACTTTTGAATGT
GAACAAAAGGAAGAAAATCAAGGAAAGAATGAGTCTAATATCAAGCCTGT
ACAGACAGTTAATATCACTGCAGGCTTTCCTGTGGTTGGTCAGAAAGATA
AGCCAGTTGATAATGCCAAATGTAGTATCAAAGGAGGCTCTAGGTTTTGT
CTATCATCTCAGTTCAGAGGCAACGAAACTGGACTCATTACTCCAAATAA
ACATGGACTTTTACAAAACCCATATCGTATACCACCACTTTTTCCCATCA
AGTCATTTGTTAAAACTAAATGTAAGAAAAATCTGCTAGAGGAAAACTTT
GAGGAACATTCAATGTCACCTGAAAGAGAAATGGGAAATGAGAACATTCC
AAGTACAGTGAGCACAATTAGCCGTAATAACATTAGAGAAAATGTTTTTA
AAGAAGCCAGCTCAAGCAATATTAATGAAGTAGGTTCCAGTACTAATGAA
GTGGGCTCCAGTATTAATGAAATAGGTTCCAGTGATGAAAACATTCAAGC
AGAACTAGGTAGAAACAGAGGGCCAAAATTGAATGCTATGCTTAGATTAG
GGGTTTTGCAACCTGAGGTCTATAAACAAAGTCTTCCTGGAAGTAATTGT
AAGCATCCTGAAATAAAAAAGCAAGAATATGAAGAAGTAGTTCAGACTGT
TAATACAGATTTCTCTCCATATCTGATTTCAGATAACTTAGAACAGCCTA
TGGGAAGTAGTCATGCATCTCAGGTTTGTTCTGAGACACCTGATGACCTG
TTAGATGATGGTGAAATAAAGGAAGATACTAGTTTTGCTGAAAATGACAT
TAAGGAAAGTTCTGCTGTTTTTAGCAAAAGCGTCCAGAAAGGAGAGCTTA
GCAGGAGTCCTAGCCCTTTCACCCATACACATTTGGCTCAGGGTTACCGA
AGAGGGGCCAAGAAATTAGAGTCCTCAGAAGAGAACTTATCTAGTGAGGA
TGAAGAGCTTCCCTGCTTCCAACACTTGTTATTTGGTAAAGTAAACAATA
TACCTTCTCAGTCTACTAGGCATAGCACCGTTGCTACCGAGTGTCTGTCT
AAGAACACAGAGGAGAATTTATTATCATTGAAGAATAGCTTAAATGACTG
CAGTAACCAGGTAATATTGGCAAAGGCATCTCAGGAACATCACCTTAGTG
AGGAAACAAAATGTTCTGCTAGCTTGTTTTCTTCACAGTGCAGTGAATTG
GAAGACTTGACTGCAAATACAAACACCCAGGATCCTTTCTTGATTGGTTC
TTCCAAACAAATGAGGCATCAGTCTGAAAGCCAGGGAGTTGGTCTGAGTG
ACAAGGAATTGGTTTCAGATGATGAAGAAAGAGGAACGGGCTTGGAAGAA
AATAATCAAGAAGAGCAAAGCATGGATTCAAACTTAGGTGAAGCAGCATC
TGGGTGTGAGAGTGAAACAAGCGTCTCTGAAGACTGCTCAGGGCTATCCT
CTCAGAGTGACATTTTAACCACTCAGCAGAGGGATACCATGCAACATAAC
CTGATAAAGCTCCAGCAGGAAATGGCTGAACTAGAAGCTGTGTTAGAACA
GCATGGGAGCCAGCCTTCTAACAGCTACCCTTCCATCATAAGTGACTCTT
CTGCCCTTGAGGACCTGCGAAATCCAGAACAAAGCACATCAGAAAAAGCA
GTATTAACTTCACAGAAAAGTAGTGAATACCCTATAAGCCAGAATCCAGA
AGGCCTTTCTGCTGACAAGTTTGAGGTGTCTGCAGATAGTTCTACCAGTA
AAAATAAAGAACCAGGAGTGGAAAGGTCATCCCCTTCTAAATGCCCATCA
TTAGATGATAGGTGGTACATGCACAGTTGCTCTGGGAGTCTTCAGAATAG
AAACTACCCATCTCAAGAGGAGCTCATTAAGGTTGTTGATGTGGAGGAGC
AACAGCTGGAAGAGTCTGGGCCACACGATTTGACGGAAACATCTTACTTG
CCAAGGCAAGATCTAGAGGGAACCCCTTACCTGGAATCTGGAATCAGCCT
CTTCTCTGATGACCCTGAATCTGATCCTTCTGAAGACAGAGCCCCAGAGT
CAGCTCGTGTTGGCAACATACCATCTTCAACCTCTGCATTGAAAGTTCCC
CAATTGAAAGTTGCAGAATCTGCCCAGAGTCCAGCTGCTGCTCATACTAC
TGATACTGCTGGGTATAATGCAATGGAAGAAAGTGTGAGCAGGGAGAAGC
CAGAATTGACAGCTTCAACAGAAAGGGTCAACAAAAGAATGTCCATGGTG
GTGTCTGGCCTGACCCCAGAAGAATTTATGCTCGTGTACAAGTTTGCCAG
AAAACACCACATCACTTTAACTAATCTAATTACTGAAGAGACTACTCATG
TTGTTATGAAAACAGATGCTGAGTTTGTGTGTGAACGGACACTGAAATAT
TTTCTAGGAATTGCGGGAGGAAAATGGGTAGTTAGCTATTTCTGGGTGAC
CCAGTCTATTAAAGAAAGAAAAATGCTGAATGAGCATGATTTTGAAGTCA
GAGGAGATGTGGTCAATGGAAGAAACCACCAAGGTCCAAAGCGAGCAAGA
GAATCCCAGGACAGAAAGATCTTCAGGGGGCTAGAAATCTGTTGCTATGG
GCCCTTCACCAACATGCCCACAGATCAACTGGAATGGATGGTACAGCTGT
GTGGTGCTTCTGTGGTGAAGGAGCTTTCATCATTCACCCTTGGCACAGGT
GTCCACCCAATTGTGGTTGTGCAGCCAGATGCCTGGACAGAGGACAATGG
CTTCCATGCAATTGGGCAGATGTGTGAGGCACCTGTGGTGACCCGAGAGT
GGGTGTTGGACAGTGTAGCACTCTACCAGTGCCAGGAGCTGGACACCTAC
CTGATACCCCAGATCCCCCACAGCCACTACTGACTGCAGCCAGCCACAGG
TACAGAGCCCAGGACCCCAAGAATGAGCTTACAAAGTGGCCTTTCCAGGC
CCTGGGAGCTCCTCTCACTCTTCAGTCCTTCTACTGTCCTGGCTACTAAA
TATTTTATGTACATCAGCCTGAAAAGGACTTCTGGCTATGCAAGGGTCCC
TTAAAGATTTTCTGCTTGAAGTCTCCCTTGGAAATCTGCCATGAGCACAA
AATTATGGTAATTTTTCACCTGAGAAGATTTTAAAACCATTTAAACGCCA
CCAATTGAGCAAGATGCTGATTCATTATTTATCAGCCCTATTCTTTCTAT
TCAGGCTGTTGTTGGCTTAGGGCTGGAAGCACAGAGTGGCTTGGCCTCAA
GAGAATAGCTGGTTTCCCTAAGTTTACTTCTCTAAAACCCTGTGTTCACA
AAGGCAGAGAGTCAGACCCTTCAATGGAAGGAGAGTGCTTGGGATCGATT
ATGTGACTTAAAGTCAGAATAGTCCTTGGGCAGTTCTCAAATGTTGGAGT
GGAACATTGGGGAGGAAATTCTGAGGCAGGTATTAGAAATGAAAAGGAAA
CTTGAAACCTGGGCATGGTGGCTCACGCCTGTAATCCCAGCACTTTGGGA
GGCCAAGGTGGGCAGATCACTGGAGGTCAGGAGTTCGAAACCAGCCTGGC
CAACATGGTGAAACCCCATCTCTACTAAAAATACAGAAATTAGCCGGTCA
TGGTGGTGGACACCTGTAATCCCAGCTACTCAGGTGGCTAAGGCAGGAGA
ATCACTTCAGCCCGGGAGGTGGAGGTTGCAGTGAGCCAAGATCATACCAC
GGCACTCCAGCCTGGGTGACAGTGAGACTGTGGCTCAAAAAAAAAAAAAA
AAAAGGAAAATGAAACTAGGAAAGGTTTCTTAAAGTCTGAGATATATTTG
CTAGATTTCTAAAGAATGTGTTCTAAAACAGCAGAAGATTTTCAAGAACC
GGTTTCCAAAGACAGTCTTCTAATTCCTCATTAGTAATAAGTAAAATGTT
TATTGTTGTAGCTCTGGTATATAATCCATTCCTCTTAAAATATAAGACCT
CTGGCATGAATATTTCATATCTATAAAATGACAGATCCCACCAGGAAGGA
AGCTGTTGCTTTCTTTGAGGTGATTTTTTTCCTTTGCTCCCTGTTGCTGA
AACCATACAGCTTCATAAATAATTTTGCTTGCTGAAGGAAGAAAAAGTGT
TTTTCATAAACCCATTATCCAGGACTGTTTATAGCTGTTGGAAGGACTAG
GTCTTCCCTAGCCCCCCCAGTGTGCAAGGGCAGTGAAGACTTGATTGTAC
AAAATACGTTTTGTAAATGTTGTGCTGTTAACACTGCAAATAAACTTGGT
AGCAAACA
HUMAN BRCA2 POLYNUCLEOTIDE SEQUENCE
(SEQ ID NO: 11)
GGTGGCGCGAGCTTCTGAAACTAGGCGGCAGAGGCGGAGCCGCTGTGGCA
CTGCTGCGCCTCTGCTGCGCCTCGGGTGTCTTTTGCGGCGGTGGGTCGCC
GCCGGGAGAAGCGTGAGGGGACAGATTTGTGACCGGCGCGGTTTTTGTCA
GCTTACTCCGGCCAAAAAAGAACTGCACCTCTGGAGCGGACTTATTTACC
AAGCATTGGAGGAATATCGTAGGTAAAAATGCCTATTGGATCCAAAGAGA
GGCCAACATTTTTTGAAATTTTTAAGACACGCTGCAACAAAGCAGATTTA
GGACCAATAAGTCTTAATTGGTTTGAAGAACTTTCTTCAGAAGCTCCACC
CTATAATTCTGAACCTGCAGAAGAATCTGAACATAAAAACAACAATTACG
AACCAAACCTATTTAAAACTCCACAAAGGAAACCATCTTATAATCAGCTG
GCTTCAACTCCAATAATATTCAAAGAGCAAGGGCTGACTCTGCCGCTGTA
CCAATCTCCTGTAAAAGAATTAGATAAATTCAAATTAGACTTAGGAAGGA
ATGTTCCCAATAGTAGACATAAAAGTCTTCGCACAGTGAAAACTAAAATG
GATCAAGCAGATGATGTTTCCTGTCCACTTCTAAATTCTTGTCTTAGTGA
AAGTCCTGTTGTTCTACAATGTACACATGTAACACCACAAAGAGATAAGT
CAGTGGTATGTGGGAGTTTGTTTCATACACCAAAGTTTGTGAAGGGTCGT
CAGACACCAAAACATATTTCTGAAAGTCTAGGAGCTGAGGTGGATCCTGA
TATGTCTTGGTCAAGTTCTTTAGCTACACCACCCACCCTTAGTTCTACTG
TGCTCATAGTCAGAAATGAAGAAGCATCTGAAACTGTATTTCCTCATGAT
ACTACTGCTAATGTGAAAAGCTATTTTTCCAATCATGATGAAAGTCTGAA
GAAAAATGATAGATTTATCGCTTCTGTGACAGACAGTGAAAACACAAATC
AAAGAGAAGCTGCAAGTCATGGATTTGGAAAAACATCAGGGAATTCATTT
AAAGTAAATAGCTGCAAAGACCACATTGGAAAGTCAATGCCAAATGTCCT
AGAAGATGAAGTATATGAAACAGTTGTAGATACCTCTGAAGAAGATAGTT
TTTCATTATGTTTTTCTAAATGTAGAACAAAAAATCTACAAAAAGTAAGA
ACTAGCAAGACTAGGAAAAAAATTTTCCATGAAGCAAACGCTGATGAATG
TGAAAAATCTAAAAACCAAGTGAAAGAAAAATACTCATTTGTATCTGAAG
TGGAACCAAATGATACTGATCCATTAGATTCAAATGTAGCACATCAGAAG
CCCTTTGAGAGTGGAAGTGACAAAATCTCCAAGGAAGTTGTACCGTCTTT
GGCCTGTGAATGGTCTCAACTAACCCTTTCAGGTCTAAATGGAGCCCAGA
TGGAGAAAATACCCCTATTGCATATTTCTTCATGTGACCAAAATATTTCA
GAAAAAGACCTATTAGACACAGAGAACAAAAGAAAGAAAGATTTTCTTAC
TTCAGAGAATTCTTTGCCACGTATTTCTAGCCTACCAAAATCAGAGAAGC
CATTAAATGAGGAAACAGTGGTAAATAAGAGAGATGAAGAGCAGCATCTT
GAATCTCATACAGACTGCATTCTTGCAGTAAAGCAGGCAATATCTGGAAC
TTCTCCAGTGGCTTCTTCATTTCAGGGTATCAAAAAGTCTATATTCAGAA
TAAGAGAATCACCTAAAGAGACTTTCAATGCAAGTTTTTCAGGTCATATG
ACTGATCCAAACTTTAAAAAAGAAACTGAAGCCTCTGAAAGTGGACTGGA
AATACATACTGTTTGCTCACAGAAGGAGGACTCCTTATGTCCAAATTTAA
TTGATAATGGAAGCTGGCCAGCCACCACCACACAGAATTCTGTAGCTTTG
AAGAATGCAGGTTTAATATCCACTTTGAAAAAGAAAACAAATAAGTTTAT
TTATGCTATACATGATGAAACATTTTATAAAGGAAAAAAAATACCGAAAG
ACCAAAAATCAGAACTAATTAACTGTTCAGCCCAGTTTGAAGCAAATGCT
TTTGAAGCACCACTTACATTTGCAAATGCTGATTCAGGTTTATTGCATTC
TTCTGTGAAAAGAAGCTGTTCACAGAATGATTCTGAAGAACCAACTTTGT
CCTTAACTAGCTCTTTTGGGACAATTCTGAGGAAATGTTCTAGAAATGAA
ACATGTTCTAATAATACAGTAATCTCTCAGGATCTTGATTATAAAGAAGC
AAAATGTAATAAGGAAAAACTACAGTTATTTATTACCCCAGAAGCTGATT
CTCTGTCATGCCTGCAGGAAGGACAGTGTGAAAATGATCCAAAAAGCAAA
AAAGTTTCAGATATAAAAGAAGAGGTCTTGGCTGCAGCATGTCACCCAGT
ACAACATTCAAAAGTGGAATACAGTGATACTGACTTTCAATCCCAGAAAA
GTCTTTTATATGATCATGAAAATGCCAGCACTCTTATTTTAACTCCTACT
TCCAAGGATGTTCTGTCAAACCTAGTCATGATTTCTAGAGGCAAAGAATC
ATACAAAATGTCAGACAAGCTCAAAGGTAACAATTATGAATCTGATGTTG
AATTAACCAAAAATATTCCCATGGAAAAGAATCAAGATGTATGTGCTTTA
AATGAAAATTATAAAAACGTTGAGCTGTTGCCACCTGAAAAATACATGAG
AGTAGCATCACCTTCAAGAAAGGTACAATTCAACCAAAACACAAATCTAA
GAGTAATCCAAAAAAATCAAGAAGAAACTACTTCAATTTCAAAAATAACT
GTCAATCCAGACTCTGAAGAACTTTTCTCAGACAATGAGAATAATTTTGT
CTTCCAAGTAGCTAATGAAAGGAATAATCTTGCTTTAGGAAATACTAAGG
AACTTCATGAAACAGACTTGACTTGTGTAAACGAACCCATTTTCAAGAAC
TCTACCATGGTTTTATATGGAGACACAGGTGATAAACAAGCAACCCAAGT
GTCAATTAAAAAAGATTTGGTTTATGTTCTTGCAGAGGAGAACAAAAATA
GTGTAAAGCAGCATATAAAAATGACTCTAGGTCAAGATTTAAAATCGGAC
ATCTCCTTGAATATAGATAAAATACCAGAAAAAAATAATGATTACATGAA
CAAATGGGCAGGACTCTTAGGTCCAATTTCAAATCACAGTTTTGGAGGTA
GCTTCAGAACAGCTTCAAATAAGGAAATCAAGCTCTCTGAACATAACATT
AAGAAGAGCAAAATGTTCTTCAAAGATATTGAAGAACAATATCCTACTAG
TTTAGCTTGTGTTGAAATTGTAAATACCTTGGCATTAGATAATCAAAAGA
AACTGAGCAAGCCTCAGTCAATTAATACTGTATCTGCACATTTACAGAGT
AGTGTAGTTGTTTCTGATTGTAAAAATAGTCATATAACCCCTCAGATGTT
ATTTTCCAAGCAGGATTTTAATTCAAACCATAATTTAACACCTAGCCAAA
AGGCAGAAATTACAGAACTTTCTACTATATTAGAAGAATCAGGAAGTCAG
TTTGAATTTACTCAGTTTAGAAAACCAAGCTACATATTGCAGAAGAGTAC
ATTTGAAGTGCCTGAAAACCAGATGACTATCTTAAAGACCACTTCTGAGG
AATGCAGAGATGCTGATCTTCATGTCATAATGAATGCCCCATCGATTGGT
CAGGTAGACAGCAGCAAGCAATTTGAAGGTACAGTTGAAATTAAACGGAA
GTTTGCTGGCCTGTTGAAAAATGACTGTAACAAAAGTGCTTCTGGTTATT
TAACAGATGAAAATGAAGTGGGGTTTAGGGGCTTTTATTCTGCTCATGGC
ACAAAACTGAATGTTTCTACTGAAGCTCTGCAAAAAGCTGTGAAACTGTT
TAGTGATATTGAGAATATTAGTGAGGAAACTTCTGCAGAGGTACATCCAA
TAAGTTTATCTTCAAGTAAATGTCATGATTCTGTTGTTTCAATGTTTAAG
ATAGAAAATCATAATGATAAAACTGTAAGTGAAAAAAATAATAAATGCCA
ACTGATATTACAAAATAATATTGAAATGACTACTGGCACTTTTGTTGAAG
AAATTACTGAAAATTACAAGAGAAATACTGAAAATGAAGATAACAAATAT
ACTGCTGCCAGTAGAAATTCTCATAACTTAGAATTTGATGGCAGTGATTC
AAGTAAAAATGATACTGTTTGTATTCATAAAGATGAAACGGACTTGCTAT
TTACTGATCAGCACAACATATGTCTTAAATTATCTGGCCAGTTTATGAAG
GAGGGAAACACTCAGATTAAAGAAGATTTGTCAGATTTAACTTTTTTGGA
AGTTGCGAAAGCTCAAGAAGCATGTCATGGTAATACTTCAAATAAAGAAC
AGTTAACTGCTACTAAAACGGAGCAAAATATAAAAGATTTTGAGACTTCT
GATACATTTTTTCAGACTGCAAGTGGGAAAAATATTAGTGTCGCCAAAGA
GTCATTTAATAAAATTGTAAATTTCTTTGATCAGAAACCAGAAGAATTGC
ATAACTTTTCCTTAAATTCTGAATTACATTCTGACATAAGAAAGAACAAA
ATGGACATTCTAAGTTATGAGGAAACAGACATAGTTAAACACAAAATACT
GAAAGAAAGTGTCCCAGTTGGTACTGGAAATCAACTAGTGACCTTCCAGG
GACAACCCGAACGTGATGAAAAGATCAAAGAACCTACTCTGTTGGGTTTT
CATACAGCTAGCGGGAAAAAAGTTAAAATTGCAAAGGAATCTTTGGACAA
AGTGAAAAACCTTTTTGATGAAAAAGAGCAAGGTACTAGTGAAATCACCA
GTTTTAGCCATCAATGGGCAAAGACCCTAAAGTACAGAGAGGCCTGTAAA
GACCTTGAATTAGCATGTGAGACCATTGAGATCACAGCTGCCCCAAAGTG
TAAAGAAATGCAGAATTCTCTCAATAATGATAAAAACCTTGTTTCTATTG
AGACTGTGGTGCCACCTAAGCTCTTAAGTGATAATTTATGTAGACAAACT
GAAAATCTCAAAACATCAAAAAGTATCTTTTTGAAAGTTAAAGTACATGA
AAATGTAGAAAAAGAAACAGCAAAAAGTCCTGCAACTTGTTACACAAATC
AGTCCCCTTATTCAGTCATTGAAAATTCAGCCTTAGCTTTTTACACAAGT
TGTAGTAGAAAAACTTCTGTGAGTCAGACTTCATTACTTGAAGCAAAAAA
ATGGCTTAGAGAAGGAATATTTGATGGTCAACCAGAAAGAATAAATACTG
CAGATTATGTAGGAAATTATTTGTATGAAAATAATTCAAACAGTACTATA
GCTGAAAATGACAAAAATCATCTCTCCGAAAAACAAGATACTTATTTAAG
TAACAGTAGCATGTCTAACAGCTATTCCTACCATTCTGATGAGGTATATA
ATGATTCAGGATATCTCTCAAAAAATAAACTTGATTCTGGTATTGAGCCA
GTATTGAAGAATGTTGAAGATCAAAAAAACACTAGTTTTTCCAAAGTAAT
ATCCAATGTAAAAGATGCAAATGCATACCCACAAACTGTAAATGAAGATA
TTTGCGTTGAGGAACTTGTGACTAGCTCTTCACCCTGCAAAAATAAAAAT
GCAGCCATTAAATTGTCCATATCTAATAGTAATAATTTTGAGGTAGGGCC
ACCTGCATTTAGGATAGCCAGTGGTAAAATCGTTTGTGTTTCACATGAAA
CAATTAAAAAAGTGAAAGACATATTTACAGACAGTTTCAGTAAAGTAATT
AAGGAAAACAACGAGAATAAATCAAAAATTTGCCAAACGAAAATTATGGC
AGGTTGTTACGAGGCATTGGATGATTCAGAGGATATTCTTCATAACTCTC
TAGATAATGATGAATGTAGCACGCATTCACATAAGGTTTTTGCTGACATT
CAGAGTGAAGAAATTTTACAACATAACCAAAATATGTCTGGATTGGAGAA
AGTTTCTAAAATATCACCTTGTGATGTTAGTTTGGAAACTTCAGATATAT
GTAAATGTAGTATAGGGAAGCTTCATAAGTCAGTCTCATCTGCAAATACT
TGTGGGATTTTTAGCACAGCAAGTGGAAAATCTGTCCAGGTATCAGATGC
TTCATTACAAAACGCAAGACAAGTGTTTTCTGAAATAGAAGATAGTACCA
AGCAAGTCTTTTCCAAAGTATTGTTTAAAAGTAACGAACATTCAGACCAG
CTCACAAGAGAAGAAAATACTGCTATACGTACTCCAGAACATTTAATATC
CCAAAAAGGCTTTTCATATAATGTGGTAAATTCATCTGCTTTCTCTGGAT
TTAGTACAGCAAGTGGAAAGCAAGTTTCCATTTTAGAAAGTTCCTTACAC
AAAGTTAAGGGAGTGTTAGAGGAATTTGATTTAATCAGAACTGAGCATAG
TCTTCACTATTCACCTACGTCTAGACAAAATGTATCAAAAATACTTCCTC
GTGTTGATAAGAGAAACCCAGAGCACTGTGTAAACTCAGAAATGGAAAAA
ACCTGCAGTAAAGAATTTAAATTATCAAATAACTTAAATGTTGAAGGTGG
TTCTTCAGAAAATAATCACTCTATTAAAGTTTCTCCATATCTCTCTCAAT
TTCAACAAGACAAACAACAGTTGGTATTAGGAACCAAAGTCTCACTTGTT
GAGAACATTCATGTTTTGGGAATAGAACAGGCTTCACCTAAAAACGTAAA
AATGGAAATTGGTAAAACTGAAACTTTTTCTGATGTTCCTGTGAAAACAA
ATATAGAAGTTTGTTCTACTTACTCCAAAGATTCAGAAAACTACTTTGAA
ACAGAAGCAGTAGAAATTGCTAAAGCTTTTATGGAAGATGATGAACTGAC
AGATTCTAAACTGCCAAGTCATGCCACACATTCTCTTTTTACATGTCCCG
AAAATGAGGAAATGGTTTTGTCAAATTCAAGAATTGGAAAAAGAAGAGGA
GAGCCCCTTATCTTAGTGGGAGAACCCTCAATCAAAAGAAACTTATTAAA
TGAATTTGACAGGATAATAGAAAATCAAGAAAAATCCTTAAAGGCTTCAA
AAAGCACTCCAGATGGCACAATAAAAGATCGAAGATTGTTTATGCATCAT
GTTTCTTTAGAGCCGATTACCTGTGTACCCTTTCGCACAACTAAGGAACG
TCAAGAGATACAGAATCCAAATTTTACCGCACCTGGTCAAGAATTTCTGT
CTAAATCTCATTTGTATGAACATCTGACTTTGGAAAAATCTTCAAGCAAT
TTAGCAGTTTCAGGACATCCATTTTATCAAGTTTCTGCTACAAGAAATGA
AAAAATGAGACACTTGATTACTACAGGCAGACCAACCAAAGTCTTTGTTC
CACCTTTTAAAACTAAATCACATTTTCACAGAGTTGAACAGTGTGTTAGG
AATATTAACTTGGAGGAAAACAGACAAAAGCAAAACATTGATGGACATGG
CTCTGATGATAGTAAAAATAAGATTAATGACAATGAGATTCATCAGTTTA
ACAAAAACAACTCCAATCAAGCAGCAGCTGTAACTTTCACAAAGTGTGAA
GAAGAACCTTTAGATTTAATTACAAGTCTTCAGAATGCCAGAGATATACA
GGATATGCGAATTAAGAAGAAACAAAGGCAACGCGTCTTTCCACAGCCAG
GCAGTCTGTATCTTGCAAAAACATCCACTCTGCCTCGAATCTCTCTGAAA
GCAGCAGTAGGAGGCCAAGTTCCCTCTGCGTGTTCTCATAAACAGCTGTA
TACGTATGGCGTTTCTAAACATTGCATAAAAATTAACAGCAAAAATGCAG
AGTCTTTTCAGTTTCACACTGAAGATTATTTTGGTAAGGAAAGTTTATGG
ACTGGAAAAGGAATACAGTTGGCTGATGGTGGATGGCTCATACCCTCCAA
TGATGGAAAGGCTGGAAAAGAAGAATTTTATAGGGCTCTGTGTGACACTC
CAGGTGTGGATCCAAAGCTTATTTCTAGAATTTGGGTTTATAATCACTAT
AGATGGATCATATGGAAACTGGCAGCTATGGAATGTGCCTTTCCTAAGGA
ATTTGCTAATAGATGCCTAAGCCCAGAAAGGGTGCTTCTTCAACTAAAAT
ACAGATATGATACGGAAATTGATAGAAGCAGAAGATCGGCTATAAAAAAG
ATAATGGAAAGGGATGACACAGCTGCAAAAACACTTGTTCTCTGTGTTTC
TGACATAATTTCATTGAGCGCAAATATATCTGAAACTTCTAGCAATAAAA
CTAGTAGTGCAGATACCCAAAAAGTGGCCATTATTGAACTTACAGATGGG
TGGTATGCTGTTAAGGCCCAGTTAGATCCTCCCCTCTTAGCTGTCTTAAA
GAATGGCAGACTGACAGTTGGTCAGAAGATTATTCTTCATGGAGCAGAAC
TGGTGGGCTCTCCTGATGCCTGTACACCTCTTGAAGCCCCAGAATCTCTT
ATGTTAAAGATTTCTGCTAACAGTACTCGGCCTGCTCGCTGGTATACCAA
ACTTGGATTCTTTCCTGACCCTAGACCTTTTCCTCTGCCCTTATCATCGC
TTTTCAGTGATGGAGGAAATGTTGGTTGTGTTGATGTAATTATTCAAAGA
GCATACCCTATACAGTGGATGGAGAAGACATCATCTGGATTATACATATT
TCGCAATGAAAGAGAGGAAGAAAAGGAAGCAGCAAAATATGTGGAGGCCC
AACAAAAGAGACTAGAAGCCTTATTCACTAAAATTCAGGAGGAATTTGAA
GAACATGAAGAAAACACAACAAAACCATATTTACCATCACGTGCACTAAC
AAGACAGCAAGTTCGTGCTTTGCAAGATGGTGCAGAGCTTTATGAAGCAG
TGAAGAATGCAGCAGACCCAGCTTACCTTGAGGGTTATTTCAGTGAAGAG
CAGTTAAGAGCCTTGAATAATCACAGGCAAATGTTGAATGATAAGAAACA
AGCTCAGATCCAGTTGGAAATTAGGAAGGCCATGGAATCTGCTGAACAAA
AGGAACAAGGTTTATCAAGGGATGTCACAACCGTGTGGAAGTTGCGTATT
GTAAGCTATTCAAAAAAAGAAAAAGATTCAGTTATACTGAGTATTTGGCG
TCCATCATCAGATTTATATTCTCTGTTAACAGAAGGAAAGAGATACAGAA
TTTATCATCTTGCAACTTCAAAATCTAAAAGTAAATCTGAAAGAGCTAAC
ATACAGTTAGCAGCGACAAAAAAAACTCAGTATCAACAACTACCGGTTTC
AGATGAAATTTTATTTCAGATTTACCAGCCACGGGAGCCCCTTCACTTCA
GCAAATTTTTAGATCCAGACTTTCAGCCATCTTGTTCTGAGGTGGACCTA
ATAGGATTTGTCGTTTCTGTTGTGAAAAAAACAGGACTTGCCCCTTTCGT
CTATTTGTCAGACGAATGTTACAATTTACTGGCAATAAAGTTTTGGATAG
ACCTTAATGAGGACATTATTAAGCCTCATATGTTAATTGCTGCAAGCAAC
CTCCAGTGGCGACCAGAATCCAAATCAGGCCTTCTTACTTTATTTGCTGG
AGATTTTTCTGTGTTTTCTGCTAGTCCAAAAGAGGGCCACTTTCAAGAGA
CATTCAACAAAATGAAAAATACTGTTGAGAATATTGACATACTTTGCAAT
GAAGCAGAAAACAAGCTTATGCATATACTGCATGCAAATGATCCCAAGTG
GTCCACCCCAACTAAAGACTGTACTTCAGGGCCGTACACTGCTCAAATCA
TTCCTGGTACAGGAAACAAGCTTCTGATGTCTTCTCCTAATTGTGAGATA
TATTATCAAAGTCCTTTATCACTTTGTATGGCCAAAAGGAAGTCTGTTTC
CACACCTGTCTCAGCCCAGATGACTTCAAAGTCTTGTAAAGGGGAGAAAG
AGATTGATGACCAAAAGAACTGCAAAAAGAGAAGAGCCTTGGATTTCTTG
AGTAGACTGCCTTTACCTCCACCTGTTAGTCCCATTTGTACATTTGTTTC
TCCGGCTGCACAGAAGGCATTTCAGCCACCAAGGAGTTGTGGCACCAAAT
ACGAAACACCCATAAAGAAAAAAGAACTGAATTCTCCTCAGATGACTCCA
TTTAAAAAATTCAATGAAATTTCTCTTTTGGAAAGTAATTCAATAGCTGA
CGAAGAACTTGCATTGATAAATACCCAAGCTCTTTTGTCTGGTTCAACAG
GAGAAAAACAATTTATATCTGTCAGTGAATCCACTAGGACTGCTCCCACC
AGTTCAGAAGATTATCTCAGACTGAAACGACGTTGTACTACATCTCTGAT
CAAAGAACAGGAGAGTTCCCAGGCCAGTACGGAAGAATGTGAGAAAAATA
AGCAGGACACAATTACAACTAAAAAATATATCTAAGCATTTGCAAAGGCG
ACAATAAATTATTGACGCTTAACCTTTCCAGTTTATAAGACTGGAATATA
ATTTCAAACCACACATTAGTACTTATGTTGCACAATGAGAAAAGAAATTA
GTTTCAAATTTACCTCAGCGTTTGTGTATCGGGCAAAAATCGTTTTGCCC
GATTCCGTATTGGTATACTTTTGCTTCAGTTGCATATCTTAAAACTAAAT
GTAATTTATTAACTAATCAAGAAAAACATCTTTGGCTGAGCTCGGTGGCT
CATGCCTGTAATCCCAACACTTTGAGAAGCTGAGGTGGGAGGAGTGCTTG
AGGCCAGGAGTTCAAGACCAGCCTGGGCAACATAGGGAGACCCCCATCTT
TACGAAGAAAAAAAAAAAGGGGAAAAGAAAATCTTTTAAATCTTTGGATT
TGATCACTACAAGTATTATTTTACAATCAACAAAATGGTCATCCAAACTC
AAACTTGAGAAAATATCTTGCTTTCAAATTGACACTA
HUMAN P-CADHERIN POLYNUCLEOTIDE SEQUENCE
(SEQ ID NO: 12)
GGCTAGCGCGGGAGGTGGAGAAAGAGGCTTGGGCGGCCCCGCTGTAGCCG
CGTGTGGGAGGACGCACGGGCCTGCTTCAAAGCTTTGGGATAACAGCGCC
TCCGGGGGATAATGAATGCGGAGCCTCCGTTTTCAGTCGACTTCAGATGT
GTCTCCACTTTTTTCCGCTGTAGCCGCAAGGCAAGGAAACATTTCTCTTC
CCGTACTGAGGAGGCTGAGGAGTGCACTGGGTGTTCTTTTCTCCTCTAAC
CCAGAACTGCGAGACAGAGGCTGAGTCCCTGTAAAGAACAGCTCCAGAAA
AGCCAGGAGAGCGCAGGAGGGCATCCGGGAGGCCAGGAGGGGTTCGCTGG
GGCCTCAACCGCACCCACATCGGTCCCACCTGCGAGGGGGCGGGACCTCG
TGGCGCTGGACCAATCAGCACCCACCTGCGCTCACCTGGCCTCCTCCCGC
TGGCTCCCGGGGGCTGCGGTGCTCAAAGGGGCAAGAGCTGAGCGGAACAC
CGGCCCGCCGTCGCGGCAGCTGCTTCACCCCTCTCTCTGCAGCCATGGGG
CTCCCTCGTGGACCTCTCGCGTCTCTCCTCCTTCTCCAGGTTTGCTGGCT
GCAGTGCGCGGCCTCCGAGCCGTGCCGGGCGGTCTTCAGGGAGGCTGAAG
TGACCTTGGAGGCGGGAGGCGCGGAGCAGGAGCCCGGCCAGGCGCTGGGG
AAAGTATTCATGGGCTGCCCTGGGCAAGAGCCAGCTCTGTTTAGCACTGA
TAATGATGACTTCACTGTGCGGAATGGCGAGACAGTCCAGGAAAGAAGGT
CACTGAAGGAAAGGAATCCATTGAAGATCTTCCCATCCAAACGTATCTTA
CGAAGACACAAGAGAGATTGGGTGGTTGCTCCAATATCTGTCCCTGAAAA
TGGCAAGGGTCCCTTCCCCCAGAGACTGAATCAGCTCAAGTCTAATAAAG
ATAGAGACACCAAGATTTTCTACAGCATCACGGGGCCGGGGGCAGACAGC
CCCCCTGAGGGTGTCTTCGCTGTAGAGAAGGAGACAGGCTGGTTGTTGTT
GAATAAGCCACTGGACCGGGAGGAGATTGCCAAGTATGAGCTCTTTGGCC
ACGCTGTGTCAGAGAATGGTGCCTCAGTGGAGGACCCCATGAACATCTCC
ATCATAGTGACCGACCAGAATGACCACAAGCCCAAGTTTACCCAGGACAC
CTTCCGAGGGAGTGTCTTAGAGGGAGTCCTACCAGGTACTTCTGTGATGC
AGATGACAGCCACAGATGAGGATGATGCCATCTACACCTACAATGGGGTG
GTTGCTTACTCCATCCATAGCCAAGAACCAAAGGACCCACACGACCTCAT
GTTCACAATTCACCGGAGCACAGGCACCATCAGCGTCATCTCCAGTGGCC
TGGACCGGGAAAAAGTCCCTGAGTACACACTGACCATCCAGGCCACAGAC
ATGGATGGGGACGGCTCCACCACCACGGCAGTGGCAGTAGTGGAGATCCT
TGATGCCAATGACAATGCTCCCATGTTTGACCCCCAGAAGTACGAGGCCC
ATGTGCCTGAGAATGCAGTGGGCCATGAGGTGCAGAGGCTGACGGTCACT
GATCTGGACGCCCCCAACTCACCAGCGTGGCGTGCCACCTACCTTATCAT
GGGCGGTGACGACGGGGACCATTTTACCATCACCACCCACCCTGAGAGCA
ACCAGGGCATCCTGACAACCAGGAAGGGTTTGGATTTTGAGGCCAAAAAC
CAGCACACCCTGTACGTTGAAGTGACCAACGAGGCCCCTTTTGTGCTGAA
GCTCCCAACCTCCACAGCCACCATAGTGGTCCACGTGGAGGATGTGAATG
AGGCACCTGTGTTTGTCCCACCCTCCAAAGTCGTTGAGGTCCAGGAGGGC
ATCCCCACTGGGGAGCCTGTGTGTGTCTACACTGCAGAAGACCCTGACAA
GGAGAATCAAAAGATCAGCTACCGCATCCTGAGAGACCCAGCAGGGTGGC
TAGCCATGGACCCAGACAGTGGGCAGGTCACAGCTGTGGGCACCCTCGAC
CGTGAGGATGAGCAGTTTGTGAGGAACAACATCTATGAAGTCATGGTCTT
GGCCATGGACAATGGAAGCCCTCCCACCACTGGCACGGGAACCCTTCTGC
TAACACTGATTGATGTCAACGACCATGGCCCAGTCCCTGAGCCCCGTCAG
ATCACCATCTGCAACCAAAGCCCTGTGCGCCAGGTGCTGAACATCACGGA
CAAGGACCTGTCTCCCCACACCTCCCCTTTCCAGGCCCAGCTCACAGATG
ACTCAGACATCTACTGGACGGCAGAGGTCAACGAGGAAGGTGACACAGTG
GTCTTGTCCCTGAAGAAGTTCCTGAAGCAGGATACATATGACGTGCACCT
TTCTCTGTCTGACCATGGCAACAAAGAGCAGCTGACGGTGATCAGGGCCA
CTGTGTGCGACTGCCATGGCCATGTCGAAACCTGCCCTGGACCCTGGAAA
GGAGGTTTCATCCTCCCTGTGCTGGGGGCTGTCCTGGCTCTGCTGTTCCT
CCTGCTGGTGCTGCTTTTGTTGGTGAGAAAGAAGCGGAAGATCAAGGAGC
CCCTCCTACTCCCAGAAGATGACACCCGTGACAACGTCTTCTACTATGGC
GAAGAGGGGGGTGGCGAAGAGGACCAGGACTATGACATCACCCAGCTCCA
CCGAGGTCTGGAGGCCAGGCCGGAGGTGGTTCTCCGCAATGACGTGGCAC
CAACCATCATCCCGACACCCATGTACCGTCCTAGGCCAGCCAACCCAGAT
GAAATCGGCAACTTTATAATTGAGAACCTGAAGGCGGCTAACACAGACCC
CACAGCCCCGCCCTACGACACCCTCTTGGTGTTCGACTATGAGGGCAGCG
GCTCCGACGCCGCGTCCCTGAGCTCCCTCACCTCCTCCGCCTCCGACCAA
GACCAAGATTACGATTATCTGAACGAGTGGGGCAGCCGCTTCAAGAAGCT
GGCAGACATGTACGGTGGCGGGGAGGACGACTAGGCGGCCTGCCTGCAGG
GCTGGGGACCAAACGTCAGGCCACAGAGCATCTCCAAGGGGTCTCAGTTC
CCCCTTCAGCTGAGGACTTCGGAGCTTGTCAGGAAGTGGCCGTAGCAACT
TGGCGGAGACAGGCTATGAGTCTGACGTTAGAGTGGTTGCTTCCTTAGCC
TTTCAGGATGGAGGAATGTGGGCAGTTTGACTTCAGCACTGAAAACCTCT
CCACCTGGGCCAGGGTTGCCTCAGAGGCCAAGTTTCCAGAAGCCTCTTAC
CTGCCGTAAAATGCTCAACCCTGTGTCCTGGGCCTGGGCCTGCTGTGACT
GACCTACAGTGGACTTTCTCTCTGGAATGGAACCTTCTTAGGCCTCCTGG
TGCAACTTAATTTTTTTTTTTAATGCTATCTTCAAAACGTTAGAGAAAGT
TCTTCAAAAGTGCAGCCCAGAGCTGCTGGGCCCACTGGCCGTCCTGCATT
TCTGGTTTCCAGACCCCAATGCCTCCCATTCGGATGGATCTCTGCGTTTT
TATACTGAGTGTGCCTAGGTTGCCCCTTATTTTTTATTTTCCCTGTTGCG
TTGCTATAGATGAAGGGTGAGGACAATCGTGTATATGTACTAGAACTTTT
TTATTAAAGAAACTTTTCCC

TABLE 2
Distribution of tumor subtypes in three breast cancer cohorts across
groups defined solely by ESR1 level
ESR1, ERBB2, and GRB7 values were downloaded for each tumor. We defined
HER-2 amplified tumors as those that had high expression levels of both ERBB2 and
GRB7. Because we did not use ESR1 expression levels to define HER-2 or BRCA1
tumors, they are not included in these tables. The remaining tumors were divided into
four groups based on the level of ESR1, where thresholds were determined relative to
each data set, and the number of samples in each of the subtypes defined by the study
authors was counted. For each study, 100% of those tumors that were identified as
either “Basal” or “Basal 1” fell into the lowest ESR1 range. In the Sørlie classified data,
over 90% of the Luminal A tumors are found in top two ESR1 groups. The largest
group of “Unknown” or non-classified tumors consistently fell in the middle ranges of
ESR1 expression.
Sørlie classification of set of 84 sporadic tumors without ERBB2 amplification^{b} in van't Veer
data.

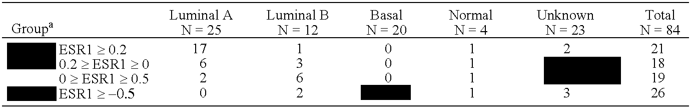

Sørlie classification of set of 97 sporadic without ERBB2 amplification and 6 non-carcinomas^{c}.

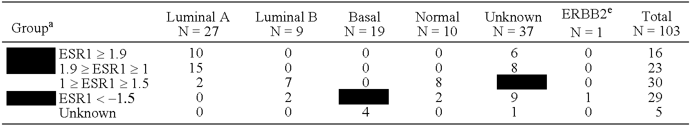

Sortiriou classification of 85 sporadic tumors without ERBB2 amplification^{d}.

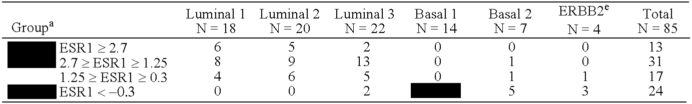

^{a}Grouping in these tables is such that the first group at the upper left of the charts (shaded black) as strong ESR1 positive, with the second group below it as moderate, the third group as weak positive, and the fourth group as ESR1 negative. Finally, groups with “Unknown” or non-classified tumors are listed as such.
^{b}van't Veer data: 78 training samples + 19 test samples − 14 ERBB2 amplified, ratio values are log₁₀.
^{c}Sørlie data: 115 tumors + 7 non malignant tissues − 18 ERBB2 amplified, ratio values are log_{2.}
^{d}Sortiriou data: 99 tumors − 14 ERBB2 amplified, ratio values are log_{2.}
^{e}Distribution for tumors classified as ERBB2 by the study authors, but not amplified for ERBB2 according to our criteria.

TABLE 3
Prognosis of 97 Sporadic Tumors by Subtype in the van't Veer Study.
The 97 patients with sporadic tumors in this cohort had invasive breast tumors
less than 5 cm (T1 or T2), no axillary metastases (N0) and were diagnosed before
the age of 55 years. Five patients received adjuvant systemic therapy. Follow-up
time in the study was at least 5 years. These 97 samples include the 78 used for
a training set and the 19 tumors used for testing their prognosis classified. ESR1
negative and ERBB2 positive subgroups were associated with the poorest prognosis
(69% and 60% respectively). The ESR1 weakly positive subtype has the best
prognosis (68%), and there is a trend toward worse prognosis with increasing ESR1
levels

| | | Good Prognosis^{a} | | Poor Prognosis^{b} | | |
|---|---|---|---|---|---|---|
| Group | Tumor Group^{c} | # Samples | % (Group) | # Samples | % (Group) | Total |
| | ESR1 Strong Positive | 12 | 57% | 9 | 42% | 21 |
| | ESR1 Mod Positive | 12 | 63% | 7 | 36% | 19 |
| | ESR1 Weak Positive | 13 | 68% | 6 | 31% | 19 |
| | ESR1 Negative | 7 | 30% | 16 | 69% | 23 |
| | ERBB2+ | 6 | 40% | 9 | 60% | 15 |
| | Total (prognosis) | 50 | | 47 | | 97 |

^{a}Good prognosis is defined as no distant metastasis in >5 years
^{b}Poor prognosis is defined as distant metastasis in <5 years
^{c}Tumor groups are defined as described as above.

1. A method of examining a test biological sample comprising a human breast cell for evidence of altered cell growth that is indicative of a breast cancer, the method comprising evaluating the levels of orphan receptor tyrosine kinase (ROR1) polynucleotides that encode the ROR1 polypeptide shown in SEQ ID NO: 2 in the biological sample, wherein an increase in the levels of the ROR1 polynucleotides in the test sample relative to a normal breast tissue sample provide evidence of altered cell growth that is indicative of a breast cancer; and wherein the levels of the ROR1 polynucleotides in the cell are evaluated by contacting the sample with a ROR1 complementary polynucleotide that hybridizes to a ROR1 nucleotide sequence shown in SEQ ID NO: 1, or a complement thereof, and evaluating the presence of a hybridization complex formed by the hybridization of the ROR1 complementary polynucleotide with the ROR1 polynucleotides in the test biological sample.

2. The method of claim 1, wherein the ROR1 complementary polynucleotide is labelled with a detectable marker.

3. The method of claim 1, wherein the presence of the hybridization complex is evaluated by Northern analysis.

4. The method of claim 1, wherein the ROR1 complementary polynucleotide comprises a primer for use in a polymerase chain reaction.

5. The method of claim 1, wherein the presence of a hybridization complex is evaluated by polymerase chain reaction.

6. The method of claim 1, wherein the ROR1 polynucleotides that are examined in the test sample are mRNA.

7. The method of claim 1, further comprising examining the expression of Her-2 (SEQ ID NO: 3), EGFR (SEQ ID NO: 4), VEGF (SEQ ID NO: 5), FMS-hike tyrosine kinase (SEQ ID NO: 6), MYC (SEQ ID NO: 7), urokinase plasminogen activator (SEQ ID NO: 8), plasminogen activator inhibitor (SEQ ID NO: 9), BRCA1 (SEQ ID NO: 10) or BRCA2 (SEQ ID NO: 11) polynucleotides in the test biological sample.

8. The method of claim 1, wherein the breast cancer is of the basal subtype.

9. The method of claim 1, wherein the breast cancer is of the BRCA 1 subtype.

10. A method of examining a test biological sample comprising a human breast cell for evidence of altered cell growth that is indicative of a breast cancer, the method comprising evaluating the levels of orphan receptor tyrosine kinase (ROR1) polypeptides having the sequence shown in SEQ ID NO: 2 in the biological sample, wherein an increase in the levels of the ROR1 polypeptides in the test sample relative to a normal breast tissue sample provide evidence of altered cell growth that is indicative of a breast cancer; and wherein the levels of the ROR1 polypeptides in the cell are evaluated by contacting the sample with an antibody that immunospecifically binds to a ROR1 polypeptide sequence shown in SEQ ID NO: 2 and evaluating the presence of a complex formed by the binding of the antibody with the ROR1 polypeptides in the sample.

11. The method of claim 10, wherein the presence of a complex is evaluated by a method selected from the group consisting of ELISA analysis, Western analysis and immunohistochemistry.

12. The method of claim 10, wherein the antibody that immunospecifically binds to a ROR1 polypeptide sequence shown in SEQ ID NO: 2 is labelled with a detectable market.

13. The method of claim 10, further comprising examining the expression of Her-2 (SEQ ID NO: 3), EGFR (SEQ ID NO: 4), VEGF (SEQ ID NO: 5), FMS-like tyrosine kinase (SEQ ID NO: 6), MYC (SEQ ID NO: 7), urokinase plasminogen activator (SEQ ID NO: 8), plasminogen activator inhibitor (SEQ ID NO: 9), BRCA1 (SEQ ID NO: 10) or BRCA2 (SEQ ID NO: 11) mRNA in the test biological sample.

14. The method of claim 10, wherein the breast cancer is of the basal subtype.

15. The method of claim 10, wherein the breast cancer is of the BRCA 1 subtype.

16. A method of examining a test human cell for evidence of a chromosomal abnormality that is indicative of a human cancer, the method comprising:
comparing orphan receptor tyrosine kinase (ROR1) polynucleotide sequences from band p31 of chromosome 1 in a normal cell to ROR1 polynucleotide sequences from band p31 of chromosome 1, band p31 on chromosome 1 in the test human cell to identify an amplification or an alteration of the ROR1 polynucleotide sequences in the test human cell, wherein an amplification or an alteration of the ROR1 polynucleotide sequences in the test human cell provides evidence of a chromosomal abnormality that is indicative of a human cancer; and
wherein chromosome 1, band p31 in the test human cell is evaluated by contacting the ROR1 polynucleotide sequences in the test human cell sample with a ROR1 complementary polynucleotide that specifically hybridizes to a ROR1 nucleotide sequence shown in SEQ ID NO: 1, or a complement thereof, and evaluating the presence of a hybridization complex formed by the hybridization of the ROR1 complementary polynucleotide with the ROR1 polynucleotide sequences in the test human cell.

17. The method of claim 16, wherein the presence of the hybridization complex is evaluated by Northern analysis, Southern analysis or polymerase chain reaction analysis.

18. The method of claim 16, wherein the cancer is breast cancer.

19. The method of claim 18, wherein the breast cancer is of the basal subtype.

20. The method of claim 18, wherein the breast cancer is of the BRCA 1 subtype.

21. A kit comprising:
a container, a label on said container, and a composition contained within said container;
wherein the composition includes a ROR1 specific antibody and/or a polynucleotide that hybridizes to a complement of the ROR1 polynucleotide shown in SEQ ID NO: 1 under stringent conditions, the label on said container indicates that the composition can be used to evaluate the presence of ROR1 protein, RNA or DNA in at least one type of mammalian cell, and instructions for using the ROR1 antibody and/or polynucleotide for evaluating the presence of ROR1 protein, RNA or DNA in at least one type of mammalian cell.
